(19) United States
(12) Patent Application Publication
(10) Pub. No.: US 2024/0270724 A1
(43) Pub. Date: Aug. 15, 2024

(54) GALECTIN-3 INHIBITING C-GLYCOSIDES

(71) Applicant: GLYCOMIMETICS, INC., Rockville, MD (US)

(72) Inventors: John L. MAGNANI, Gaithersburg, MD (US); John M. PETERSON, Slate Hill, NY (US); Yusuf U. VOHRA, Germantown, MD (US); lndranath GHOSH, Olney, MD (US); Jason NOGUEIRA, Germantown, MD (US); Arun K. SARKAR, North Potomac, MD (US); Debatosh MAJUMDAR, Germantown, MD (US)

(73) Assignee: GLYCOMIMETICS, INC., Rockville, MD (US)

(21) Appl. No.: 18/560,441

(22) PCT Filed: May 13, 2022
(86) PCT No.: PCT/US2022/029132

§ 371 Date: Nov. 13, 2023

Related U.S. Application Data
(60) Provisional application No. 63/296,555, filed on Jan. 5, 2022.
(60) Provisional application No. 63/201,815, filed on May 14, 2021.

(51) Int. Cl. C07D 405/04; A61K 31/4192

(52) U.S. Cl. CPC C07D 405/04; A61K 31/4192

(57) ABSTRACT

Compounds of formula (I), compositions, and methods for treatment and/or prevention of at least one disease, disorder, and/or condition by inhibiting binding of galectin-3 to a ligand are disclosed. For example, inhibitors of galectin-3 are described and pharmaceutical compositions comprising at least one such inhibitor are described.

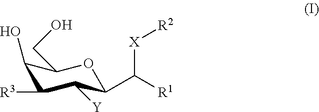

This application claims the benefit under 35 U.S.C. § 119(e) to U.S. Provisional Application No. 63/201,815 filed May 14, 2021, and U.S. Provisional Application No. 63/296,555 filed Jan. 5, 2022, which applications are incorporated by reference herein in their entirety.

Compounds, compositions, and methods for treating and/or preventing at least one disease, disorder, and/or condition associated with galectin-3 activity including, for example, inflammatory diseases, fibrosis, and cancers, are disclosed herein.

When a tissue is infected or damaged, the inflammatory process directs leukocytes and other immune system components to the site of infection or injury. Within this process, leukocytes play an important role in the engulfment and digestion of microorganisms. The recruitment of leukocytes to infected or damaged tissue is critical for mounting an effective immune defense.

Galectins are proteins with a characteristic carbohydrate recognition domain (CRD) (Barondes, S. H., Cooper, D. N. W., Gitt, M. A., and Leffler, H. (1994). Galectins. Structure and function of a large family of animal lectins. J. Biol. Chem. 269:20807-20810; Leffler, H., Carlsson, S., Hedlund, M., Qian, Y. and Poirier, F. (2004) Introduction to galectins. Glycoconj. J. 19: 433-440). Galectin subunits can contain either one or two CRDs within a single peptide chain. The mono-CRDs galectins can occur as monomers or dimers in vertebrates. Galectin-3 is a monomer in solution but may aggregate and become multimeric upon encounter with ligands. Galectins are synthesized as cytosolic proteins. Evidence suggests roles for galectins in inflammation, fibrosis, cancer, and other disorders (see, e.g., U.S. Pat. No. 7,638,623).

A pro-inflammatory role of galectin-3 is indicated by its induction in cells at inflammatory sites, effects on immune cells, and decrease of the inflammatory response shown in animal models. Inflammation is a protective response of the body to invading organisms and tissue injury. However, if unbalanced, frequently it is also destructive and occurs as part of the pathology of many diseases. Because of this, there is great medical interest in pharmacological modulation of galectin-3 mediated inflammation.

Immunohistochemical studies show changed expression of certain galectins in cancer. Direct evidence for a role of galectin-3 in cancer comes from mouse models. In paired tumor cell lines (with decreased or increased expression of galectin-3), the induction of galectin-3 gives more tumors and metastasis, while suppression of galectin-3 gives less tumors and metastasis. Galectin-3 has been proposed to enhance tumor growth by being anti-apoptotic, to promote angiogenesis, or to promote metastasis by affecting cell adhesion.

Both natural and synthetic modulators of galectin-3 have been identified. However, natural compounds that have been identified as galectin-3 ligands are not suitable for use as active components in pharmaceutical compositions because they have been reported to have low activity and specificity for galectins and galectin-3. As natural products, they are difficult to produce as well-characterized drugs and are susceptible to acidic hydrolysis in the stomach and to enzymatic degradation. In addition, previously identified natural galectin-3 modulators are large and hydrophilic in nature and are not readily absorbed from the gastrointestinal tract following oral administration.

Accordingly, there is a need in the art for inhibitors of galectin-3 mediated function. The present disclosure may fulfill this need and/or may provide other advantages.

Compounds, compositions, and methods for treating and/or preventing (i.e., reducing the likelihood of occurrence or reoccurrence of) at least one disease, disorder, and/or condition in which inhibiting binding of galectin-3 to one or more ligands may play a role are disclosed.

Disclosed is at least one compound chosen from compounds of Formula (I):

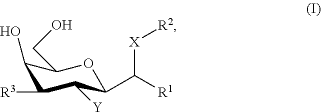

prodrugs of compounds of Formula (I), and pharmaceutically acceptable salts of any of the foregoing, wherein R¹, R², R³, X, and Y are as defined herein.

As used herein, “compound of Formula (I)” includes compounds of Formula (I), pharmaceutically acceptable salts of compounds of Formula (I), prodrugs of compounds of Formula (I), and pharmaceutically acceptable salts of prodrugs of compounds of Formula (I).

In some embodiments, compositions comprising at least one compound of Formula (I) and optionally at least one additional pharmaceutically acceptable ingredient are presented. In some embodiments, the compositions are pharmaceutical compositions.

In some embodiments, a method for treatment and/or prevention of at least one disease, disorder, and/or condition where inhibition of galectin-3 mediated functions is useful is disclosed, the method comprising administering to a subject in need thereof an effective amount of at least one compound of Formula (I) and/or a pharmaceutical composition comprising at least one compound of Formula (I).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the synthesis of Compound 19.

FIG. 2 is a diagram illustrating the synthesis of Compound 27.

FIG. 3 is a diagram illustrating the synthesis of Compound 35.

FIG. 4A is a diagram illustrating the prophetic synthesis of Compound 43.

FIG. 4B is a diagram illustrating the synthesis of Compound 43.

FIG. 5 is a diagram illustrating the prophetic synthesis of Compound 58.

Disclosed herein are inhibitors of galectin-3, pharmaceutical compositions comprising the same, and methods for inhibiting galectin-3 mediated functions using the same. The compounds and compositions of the present disclosure may be useful for treating and/or preventing at least one disease, disorder, and/or condition that is treatable by inhibiting binding of galectin-3 to one or more ligands.

In some embodiments, disclosed is at least one compound chosen from compounds of Formula (I):

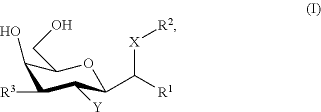

prodrugs of compounds of Formula (I), and pharmaceutically acceptable salts of any of the foregoing, wherein:
R¹ is chosen from —CN, —CH₂CN, and —C(═O)Q groups, wherein Q is chosen from —OZ¹, —NHOH—, —NHOCH₃, —NHCN, and —NZ¹Z² groups, wherein Z¹ and Z², which may be identical or different, are independently chosen from H, C₁₋₈ alkyl, C₁₋₈ haloalkyl, C₁₋₈ deuteroalkyl, C₂₋₁₂ heterocyclyl, C₆₋₁₈ aryl, and C₁₋₁₃ heteroaryl groups, wherein the C₂₋₁₂ heterocyclyl, C₆₋₁₈ aryl, and C₁₋₁₃ heteroaryl groups are optionally substituted with one or more groups independently chosen from halo, C₁₋₈ alkyl, C₁₋₈ hydroxyalkyl, C₁₋₈ haloalkyl, C₆₋₁₈ aryl, —OT¹, —C(═O)OT¹, —C(═O)NT¹T², —CN, —ST¹, —S(O)T¹, and —SO₂T¹ groups, wherein T¹ and T², which may be identical or different, are independently chosen from H, C₁₋₈ alkyl, and C₁₋₈ haloalkyl groups, or T and T² join together along with the nitrogen atom to which they are attached to form a ring, or Z¹ and Z² join together along with the nitrogen atom to which they are attached to form a ring;
R² is chosen from C₇₋₁₉ arylalkyl groups substituted with one or more bromo and optionally substituted with one or more groups independently chosen from fluoro, chloro, C₁₋₈ alkyl, C₁₋₈ hydroxyalkyl, C₁₋₈ haloalkyl, C₆₋₁₈ aryl, —OZ³, —C(═O)OZ³, —C(═O)NZ³Z⁴, and —SO₂Z³ groups, wherein Z¹ and Z⁴, which may be identical or different, are independently chosen from H, C₁₋₈ alkyl, and C₁₋₈ haloalkyl groups, or Z³ and Z⁴ join together along with the nitrogen atom to which they are attached to form a ring;
R³ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R⁴, C₁₋₈ alkyl, C₁₋₈ haloalkyl, —C(═O)OZ⁵, and —C(═O)NZ⁵Z⁶ groups, wherein R⁴ is independently chosen from C₆₋₁₈ aryl groups optionally substituted with one or more groups independently chosen from halo, C₁₋₈ alkyl, —OZ⁷, —C(═O)OZ⁷, and —C(═O)NZ⁷Z⁸ groups, wherein Z⁵, Z⁶, Z⁷, and Z⁸, which may be identical or different, are independently chosen from H and C₁₋₄ alkyl groups, or Z⁵ and Z⁶ join together along with the nitrogen atom to which they are attached to form a ring and/or Z⁷ and Z⁸ join together along with the nitrogen atom to which they are attached to form a ring;
X is chosen from —O—, —S—, —CH₂—, and —N(R⁵)—, wherein R⁵ is chosen from H, C₁₋₈ alkyl, C₂₋₄ alkenyl, C₂₋₈ alkynyl, C₁₋₈ haloalkyl, C₂₋₈ haloalkenyl, and C₂₋₈ haloalkynyl groups;
Y is chosen from H, halo, and —OZ⁹ groups, wherein Z⁹ is chosen from H, C₁₋₈ alkyl, C₁₋₈ haloalkyl, and C₁₋₈ deuteroalkyl groups; and
wherein each of Z¹, Z², Z³, Z⁴, Z⁵, Z⁶, Z⁷, Z⁸, Z⁹, T¹, and T² is optionally substituted with one or more groups independently chosen from halo and —OR⁶ groups, wherein R⁶ is independently chosen from H and C₁₋₈ alkyl groups.

In some embodiments, the compound of Formula (I) is chosen from compounds of Formula (IA):

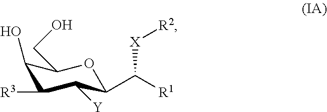

prodrugs of Formula (IA), and pharmaceutically acceptable salts of any of the foregoing.

In some embodiments, the compound of Formula (I) is chosen from compounds of Formula (IA).

In some embodiments, the compound of Formula (I) is chosen from compounds of Formula (IB):

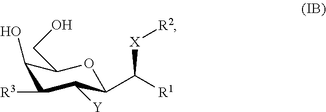

prodrugs of Formula (IB), and pharmaceutically acceptable salts of any of the foregoing.

In some embodiments, the compound of Formula (I) is chosen from compounds of Formula (IB).

In some embodiments, R¹ is chosen from —CN, —CH₂CN, and —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H, C₁₋₈ alkyl, C₂₋₁₂ heterocyclyl, C₆₋₁₈ aryl, and C₁₋₁₃ heteroaryl groups, wherein the C₂₋₁₂ heterocyclyl, C₆₋₁₈ aryl, and C₁₋₁₃ heteroaryl groups are optionally substituted with one or more groups independently chosen from halo, C₁₋₈ alkyl, C₁₋₈ hydroxyalkyl, C₁₋₈ haloalkyl, C₆₋₁₈ aryl, —OT¹, —C(═O)OT¹, —C(═O)NT¹T², —CN, —ST¹, —S(O)T¹, and —SO₂T¹ groups, wherein T¹ and T², which may be identical or different, are independently chosen from H, C₁₋₈ alkyl, and C₁₋₈ haloalkyl groups, or T¹ and T² join together along with the nitrogen atom to which they are attached to form a ring, or Z¹ and Z² join together along with the nitrogen atom to which they are attached to form a ring.

In some embodiments, R¹ is chosen from —CN, —CH₂CN, and —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H and C₁₋₈ alkyl groups, or Z¹ and Z² join together along with the nitrogen atom to which they are attached to form a ring.

In some embodiments, R¹ is chosen from —CN, —CH₂CN, and —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H and C₁₋₈ haloalkyl groups, or Z¹ and Z² join together along with the nitrogen atom to which they are attached to form a ring.

In some embodiments, R¹ is chosen from —CN, —CH₂CN, and —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H and C₁₋₈ deuteroalkyl groups, or Z¹ and Z² join together along with the nitrogen atom to which they are attached to form a ring.

In some embodiments, R¹ is chosen from —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H and C₁₋₈ alkyl groups, or Z¹ and Z² join together along with the nitrogen atom to which they are attached to form a ring.

In some embodiments, R¹ is chosen from —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H and C₁₋₈ haloalkyl groups, or Z¹ and Z² join together along with the nitrogen atom to which they are attached to form a ring.

In some embodiments, R¹ is chosen from —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H and C₁₋₈ deuteroalkyl groups, or Z¹ and Z² join together along with the nitrogen atom to which they are attached to form a ring.

In some embodiments, R¹ is chosen from —C(═O)Q groups, wherein Q is chosen from —OZ¹ and —NZ¹Z² groups. In some embodiments, R¹ is chosen from —C(═O)Q groups, wherein Q is chosen from —OZ¹ groups. In some embodiments, R¹ is chosen from —C(═O)Q groups, wherein Q is chosen from —NZ¹Z² groups.

In some embodiments, R¹ is chosen from —C(═O)Q groups, wherein Q is chosen from —NZ¹Z² groups, wherein Z¹ and Z², which may be identical or different, are independently chosen from H, C₁₋₈ alkyl, C₁₋₈ haloalkyl, C₁₋₈ deuteroalkyl, C₂₋₁₂ heterocyclyl, C₆₋₁₈ aryl, and C₁₋₁₃ heteroaryl groups.

In some embodiments, R¹ is chosen from —C(═O)Q groups, wherein Q is chosen from —NZ¹Z² groups, wherein Z¹ and Z², which may be identical or different, are independently chosen from H, C₁₋₈ alkyl, C₂₋₁₂ heterocyclyl, C₆₋₁₈ aryl, and C₁₋₁₃ heteroaryl groups.

In some embodiments, R¹ is chosen from —C(═O)Q groups, wherein Q is chosen from —NZ¹Z² groups, wherein Z¹ and Z², which may be identical or different, are independently chosen from H, C₁₋₈ alkyl, C₁₋₈ haloalkyl, and C₁₋₈ deuteroalkyl groups.

In some embodiments, R¹ is chosen from —C(═O)Q groups, wherein Q is chosen from —NZ¹Z² groups, wherein Z¹ and Z², which may be identical or different, are independently chosen from H and C₁₋₈ alkyl groups.

In some embodiments, R¹ is chosen from —C(═O)Q groups, wherein Q is chosen from —NZ¹Z² groups, wherein Z¹ and Z², which may be identical or different, are independently chosen from H and C₁₋₈ haloalkyl groups.

In some embodiments, R¹ is chosen from —C(═O)Q groups, wherein Q is chosen from —NZ¹Z² groups, wherein Z¹ and Z², which may be identical or different, are independently chosen from H and C₁₋₈ deuteroalkyl groups.

In some embodiments, R¹ is chosen from —C(═O)Q groups, wherein Q is chosen from —NZ¹Z² groups, wherein Z¹ and Z², which may be identical or different, are independently chosen from H, C₂₋₁₂ heterocyclyl, C₆₋₁₈ aryl, and C₁₋₁₃ heteroaryl groups.

In some embodiments, R¹ is chosen from —C(═O)Q groups, wherein Q is chosen from —NZ¹Z² groups, wherein Z¹ and Z², which may be identical or different, are independently chosen from H and C₂₋₁₂ heterocyclyl groups.

In some embodiments, R¹ is chosen from —C(═O)Q groups, wherein Q is chosen from —NZ¹Z² groups, wherein Z¹ and Z², which may be identical or different, are independently chosen from H and C₆₋₁₈ aryl groups.

In some embodiments, R¹ is chosen from —C(═O)Q groups, wherein Q is chosen from —NZ¹Z² groups, wherein Z¹ and Z², which may be identical or different, are independently chosen from H and C₁₋₁₃ heteroaryl groups.

In some embodiments, at least one of Z¹ and Z² is H. In some embodiments, each of Z¹ and Z² is H. In some embodiments, only one of Z¹ and Z² is H. In some embodiments, at least one of Z¹ and Z², which may be identical or different, is independently chosen from C₁₋₈ alkyl, C₁₋₈ haloalkyl, C₁₋₈ deuteroalkyl, C₂₋₁₂ heterocyclyl, C₆₋₁₈ aryl, and C₁₋₁₃ heteroaryl groups. In some embodiments, at least one of Z¹ and Z², which may be identical or different, is independently chosen from C₁₋₈ alkyl, C₂₋₁₂ heterocyclyl, C₆₋₁₈ aryl, and C₁₋₁₃ heteroaryl groups. In some embodiments, Z¹ is H and Z² is chosen from C₁₋₈ alkyl, C₁₋₈ haloalkyl, C₁₋₈ deuteroalkyl, C₂₋₁₂ heterocyclyl, C₆₋₁₈ aryl, and C₁₋₁₃ heteroaryl groups. In some embodiments, Z¹ is H and Z² is chosen from C₁₋₈ alkyl, C₂₋₁₂ heterocyclyl, C₆₋₁₈ aryl, and C₁₋₁₃ heteroaryl groups. In some embodiments, Z¹ is H and Z² is chosen from C₁₋₈ alkyl groups. In some embodiments, Z¹ is H and Z² is chosen from C₁₋₈ haloalkyl groups. In some embodiments, Z¹ is H and Z² is chosen from C₁₋₈ deuteroalkyl groups. In some embodiments, Z¹ is H and Z² is chosen from C₂₋₁₂ heterocyclyl groups. In some embodiments, Z¹ is H and Z² is chosen from C₆₋₁₈ aryl groups. In some embodiments, Z¹ is H and Z² is chosen from C₁₋₃ heteroaryl groups.

In some embodiments, at least one of Z¹ and Z², which may be identical or different, is independently chosen from C₁₋₈ alkyl groups. In some embodiments, each of Z¹ and Z², which may be identical or different, is independently chosen from C₁₋₈ alkyl groups. In some embodiments, only one of Z¹ and Z² is chosen from C₁₋₈ alkyl groups. In some embodiments, at least one of Z¹ and Z², which may be identical or different, is independently chosen from C₁₋₆ alkyl groups. In some embodiments, each of Z¹ and Z², which may be identical or different, is independently chosen from C₁₋₆ alkyl groups. In some embodiments, only one of Z¹ and Z² is chosen from C₁₋₆ alkyl groups. In some embodiments, at least one of Z¹ and Z², which may be identical or different, is independently chosen from C₁₋₄ alkyl groups. In some embodiments, each of Z¹ and Z², which may be identical or different, is independently chosen from C₁₋₄ alkyl groups. In some embodiments, only one of Z¹ and Z² is chosen from C₁₋₄ alkyl groups.

In some embodiments, at least one of Z¹ and Z², which may be identical or different, is independently chosen from methyl, ethyl, propyl, and butyl groups. In some embodiments, at least one of Z¹ and Z, which may be identical or different, is independently chosen from methyl, ethyl, n-propyl, isopropyl, cyclopropyl, 1-methylcyclopropyl, n-butyl, sec-butyl, isobutyl, and tert-butyl. In some embodiments, each of Z¹ and Z², which may be identical or different, is independently chosen from methyl, ethyl, propyl, and butyl groups. In some embodiments, each of Z¹ and Z², which may be identical or different, is independently chosen from methyl, ethyl, in-propyl, isopropyl, cyclopropyl, 1-methylcyclopropyl, n-butyl, sec-butyl, isobutyl, and tert-butyl. In some embodiments, only one of Z¹ and Z² is chosen from methyl, ethyl, propyl, and butyl groups. In some embodiments, only one of Z¹ and Z² is chosen from methyl, ethyl, n-propyl, isopropyl, cyclopropyl, 1-methylcyclopropyl, i-butyl, sec-butyl, isobutyl, and tert-butyl. In some embodiments, Z¹ is H and Z² is chosen from methyl, ethyl, propyl, and butyl groups. In some embodiments, Z¹ is H and Z² is chosen from methyl, ethyl, i-propyl, isopropyl, cyclopropyl, 1-methylcyclopropyl, n-butyl, sec-butyl, isobutyl, and tert-butyl groups.

In some embodiments, at least one of Z¹ and Z², which may be identical or different, is independently chosen from C₁₋₈ haloalkyl groups. In some embodiments, each of Z¹ and Z², which may be identical or different, is independently chosen from C₁₋₈ haloalkyl groups. In some embodiments, only one of Z¹ and Z² is chosen from C₁₋₈ haloalkyl groups. In some embodiments, at least one of Z¹ and Z², which may be identical or different, is independently chosen from C₁₋₆ haloalkyl groups. In some embodiments, each of Z¹ and Z², which may be identical or different, is independently chosen from C₁₋₆ haloalkyl groups. In some embodiments, only one of Z¹ and Z² is chosen from C₁₋₆ alkyl groups. In some embodiments, at least one of Z¹ and Z², which may be identical or different, is independently chosen from C₁₋₄ haloalkyl groups. In some embodiments, each of Z¹ and Z², which may be identical or different, is independently chosen from C₁₋₄ haloalkyl groups. In some embodiments, only one of Z¹ and Z² is chosen from C₁₋₄ haloalkyl groups. In some embodiments, Z¹ is H and Z² is chosen from C₁₋₄ haloalkyl groups. In some embodiments, the C₁₋₄ haloalkyl group is chosen from fluoroalkyl groups. In some embodiments, the fluoroalkyl group is chosen from trifluoromethyl, difluoromethyl, monofluoromethyl, 1-trifluoromethylcyclopropyl, 2,2,2-trifluoroethyl, 2,2-difluoroethyl, 2-monofluoroethyl, and 1,2-difluoroethyl. In some embodiments, Z¹ is H and Z² is 1-trifluoromethylcyclopropyl.

In some embodiments, at least one of Z¹ and Z², which may be identical or different, is independently chosen from C₁₋₈ deuteroalkyl groups. In some embodiments, each of Z¹ and Z², which may be identical or different, is independently chosen from C₁₋₈ deuteroalkyl groups. In some embodiments, only one of Z¹ and Z² is chosen from C₁₋₈ deuteroalkyl groups. In some embodiments, the C₁₋₈ deuteroalkyl group is chosen from monodeuteroalkyl, dideuteroalkyl, trideuteroalkyl, tetradeuteroalkyl, pentadeuteroalkyl, hexadeuteroalkyl, heptadeuteroalkyl, octadeuteroalkyl nonadeuteroalkyl, and decadeuteroalkyl groups. In some embodiments, the C₁₋₈ deuteroalkyl group is chosen from trideuteroalkyl groups. In some embodiments, the C₁₋₈ deuteroalkyl group is chosen from perdeuteroalkyl groups. In some embodiments, at least one of Z¹ and Z², which may be identical or different, is independently chosen from C₁₋₆ deuteroalkyl groups. In some embodiments, each of Z¹ and Z², which may be identical or different, is independently chosen from C₁₋₆ deuteroalkyl groups. In some embodiments, only one of Z¹ and Z² is chosen from C₁₋₆ deuteroalkyl groups. In some embodiments, the C₁₋₆ deuteroalkyl group is chosen from monodeuteroalkyl, dideuteroalkyl, trideuteroalkyl, tetradeuteroalkyl, pentadeuteroalkyl, hexadeuteroalkyl, heptadeuteroalkyl, octadeuteroalkyl nonadeuteroalkyl, and decadeuteroalkyl groups. In some embodiments, the C₁₋₆ deuteroalkyl group is chosen from trideuteroalkyl groups. In some embodiments, the C₁₋₆ deuteroalkyl group is chosen from perdeuteroalkyl groups. In some embodiments, at least one of Z¹ and Z², which may be identical or different, is independently chosen from C₁₋₄ deuteroalkyl groups. In some embodiments, each of Z¹ and Z², which may be identical or different, is independently chosen from C₁₋₄ deuteroalkyl groups. In some embodiments, only one of Z¹ and Z² is chosen from C₁₋₄ deuteroalkyl groups. In some embodiments, the C₁₋₄ deuteroalkyl group is chosen from monodeuteroalkyl, dideuteroalkyl, trideuteroalkyl, tetradeuteroalkyl, pentadeuteroalkyl, hexadeuteroalkyl, heptadeuteroalkyl, octadeuteroalkyl, and nonadeuteroalkyl groups. In some embodiments, the C₁₋₄ deuteroalkyl group is chosen from trideuteroalkyl groups. In some embodiments, the C₁₋₄ deuteroalkyl group is chosen from perdeuteroalkyl groups.

In some embodiments, at least one of Z¹ and Z², which may be identical or different, is independently chosen from —CHD₂, —CD₃, —CHDCH₂D, —CH₂CD₃, —CD₂CD₃, —CD₂CD₂CD₃, and —CD(CD₃)₂. In some embodiments, each of Z¹ and Z², which may be identical or different, is independently chosen from —CHD₂, —CD₃, —CHDCH₂D, —CH₂CD₃, —CD₂CD₃, —CD₂CD₂CD₃, and —CD(CD₃)₂. In some embodiments, only one of Z¹ and Z² is chosen from —CHD₂, —CD₃, —CHDCH₂D, —CH₂CD₃, —CD₂CD₃, —CD₂CD₂CD₃, and —CD(CD₃)₂. In some embodiments, Z¹ is H and Z² is chosen from —CHD₂, —CD₃, —CHDCH₂D, —CH₂CD₃, —CD₂CD₃, —CD₂CD₂CD₃, and —CD(CD₃)₂. In some embodiments, Z¹ is H and Z² is —CD(CD₃)₂.

In some embodiments, at least one of Z¹ and Z², which may be identical or different, is independently chosen from C₂₋₁₂ heterocyclyl groups. In some embodiments, at least one of Z¹ and Z², which may be identical or different, is independently chosen from C₂₋₈ heterocyclyl groups. In some embodiments, at least one of Z¹ and Z², which may be identical or different, is independently chosen from C₄₋₆ heterocyclyl groups.

In some embodiments, at least one of Z¹ and Z², which may be identical or different, is independently chosen from C₆₋₁₈ aryl groups. In some embodiments, at least one of Z¹ and Z², which may be identical or different, is independently chosen from C₆₋₁₂ aryl groups. In some embodiments, at least one of Z¹ and Z², which may be identical or different, is independently chosen from C₆₋₁₀ aryl groups.

In some embodiments, at least one of Z¹ and Z², which may be identical or different, is independently chosen from C₁₋₁₃ heteroaryl groups. In some embodiments, at least one of Z¹ and Z², which may be identical or different, is independently chosen from C₁₋₉ heteroaryl groups. In some embodiments, at least one of Z¹ and Z², which may be identical or different, is independently chosen from C₁₋₅ heteroaryl groups.

In some embodiments, R¹ is chosen from

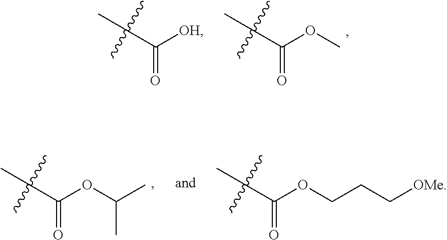

In some embodiments, R¹ is

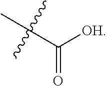 [SMILES: *C(=O)O]

In some embodiments, R¹ is

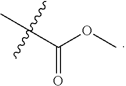 [SMILES: *C(=O)OC]

In some embodiments, R¹ is

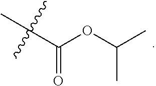 [SMILES: *C(=O)OC(C)C]

In some embodiments, R¹ is

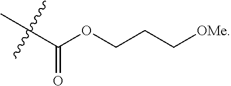 [SMILES: *C(=O)OCCCOC]

In some embodiments, R¹ is chosen from

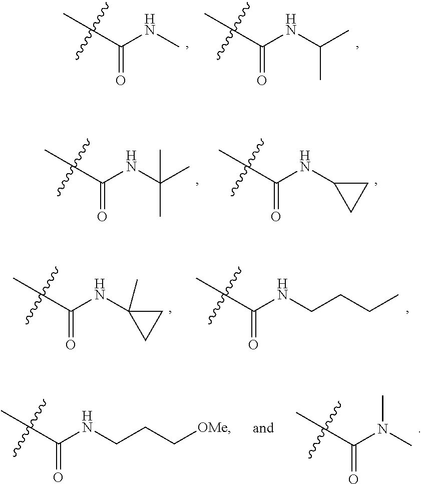

In some embodiments, R¹ is

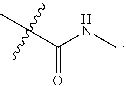 [SMILES: *C(=O)NC]

In some embodiments, R¹ is

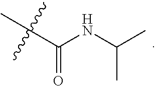 [SMILES: *C(=O)NC(C)C]

In some embodiments, R¹ is

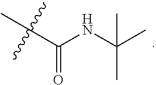 [SMILES: *C(=O)NC(C)(C)C]

In some embodiments, R¹ is

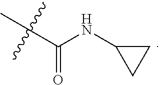 [SMILES: *C(=O)NC1CC1]

In some embodiments, R¹ is

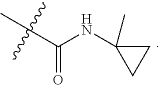 [SMILES: *C(=O)NC1(C)CC1]

In some embodiments, R¹ is

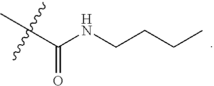 [SMILES: *C(=O)NCCCC]

In some embodiments, R¹ is

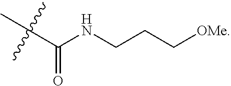 [SMILES: *C(=O)NCCCOC]

In some embodiments, R¹ is

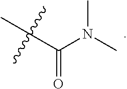 [SMILES: *C(=O)N(C)C]

In some embodiments, R¹ is chosen from

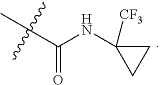 [SMILES: *C(=O)NC1(C(F)(F)F)CC1]

In some embodiments, R¹ is chosen from

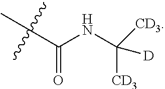 [SMILES: *C(=O)NC([2H])(C([2H])([2H])[2H])C([2H])([2H])[2H]]

In some embodiments, R¹ is chosen from

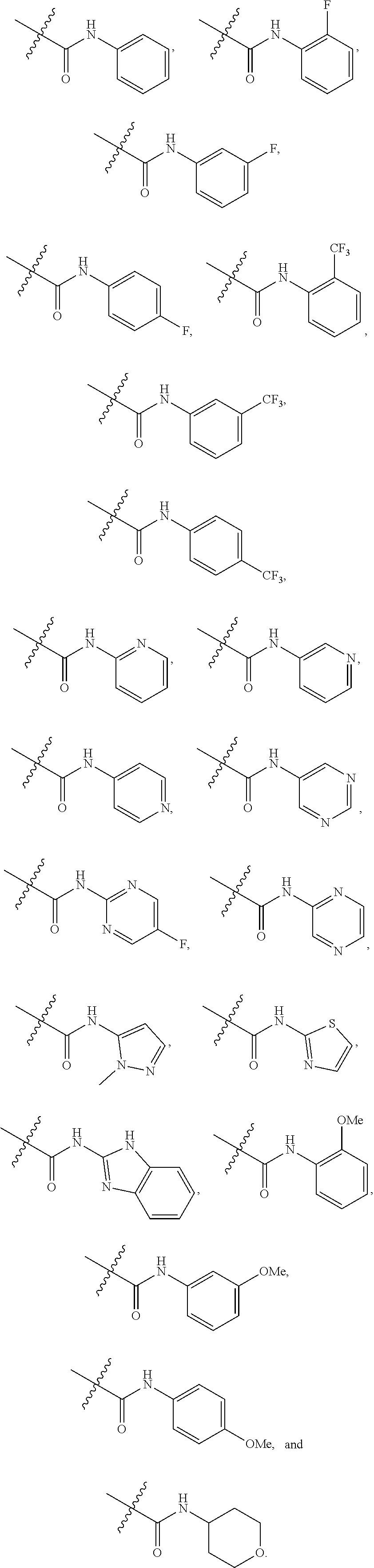

In some embodiments, R is

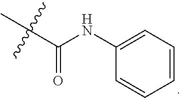 [SMILES: *C(=O)Nc1ccccc1]

In some embodiments, R¹ is

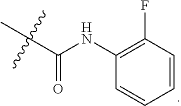 [SMILES: *C(=O)Nc1ccccc1F]

In some embodiments, R¹ is

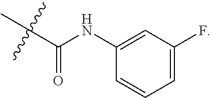 [SMILES: *C(=O)Nc1cccc(F)c1]

In some embodiments, R¹ is

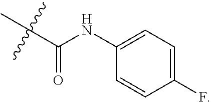 [SMILES: *C(=O)Nc1ccc(F)cc1]

In some embodiments, R¹ is

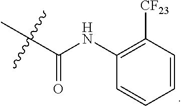

In some embodiments, R¹ is

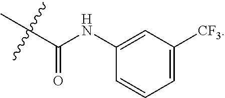 [SMILES: *C(=O)Nc1cccc(C(F)(F)F)c1]

In some embodiments, R¹ is

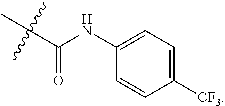 [SMILES: *C(=O)Nc1ccc(C(F)(F)F)cc1]

In some embodiments, R¹ is

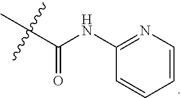 [SMILES: *C(=O)Nc1ccccn1]

In some embodiments, R¹ is

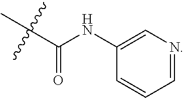 [SMILES: *C(=O)Nc1cccnc1]

In some embodiments, R¹ is

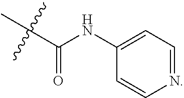 [SMILES: *C(=O)Nc1ccncc1]

In some embodiments, R¹ is

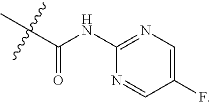 [SMILES: *C(=O)Nc1ncc(F)cn1]

In some embodiments, R¹ is

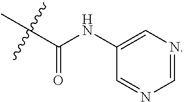 [SMILES: *C(=O)Nc1cncnc1]

In some embodiments, R¹ is

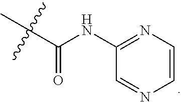 [SMILES: *C(=O)Nc1cnccn1]

In some embodiments, R¹ is

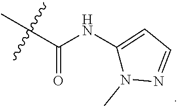 [SMILES: *C(=O)Nc1ccnn1C]

In some embodiments, R¹ is

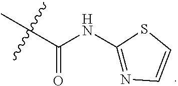 [SMILES: *C(=O)Nc1nccs1]

In some embodiments, R¹ is

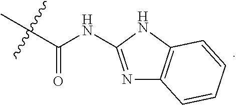 [SMILES: *C(=O)Nc1nc2ccccc2[nH]1]

In some embodiments, R¹ is

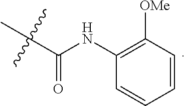 [SMILES: *C(=O)Nc1ccccc1OC]

In some embodiments, R¹ is

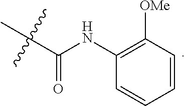 [SMILES: *C(=O)Nc1ccccc1OC]

In some embodiments, R¹ is

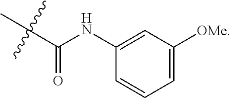 [SMILES: *C(=O)Nc1cccc(OC)c1]

In some embodiments, R¹ is

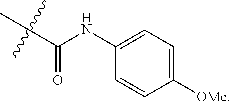 [SMILES: *C(=O)Nc1ccc(OC)cc1]

In some embodiments, R¹ is

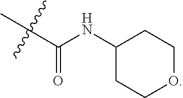 [SMILES: *C(=O)NC1CCOCC1]

In some embodiments, R² is chosen from C₇₋₁₅ arylalkyl groups substituted with one or more bromo. In some embodiments, R² is chosen from C₇₋₁₁ arylalkyl groups substituted with one or more bromo.

In some embodiments, R² is chosen from C₇₋₁₉ arylalkyl groups substituted with one bromo. In some embodiments, R² is chosen from C₇₋₁₉ arylalkyl groups substituted with two bromo. In some embodiments, R² is chosen from C₇₋₁₉ arylalkyl groups substituted with three bromo.

In some embodiments, R² is chosen from C₇₋₁₉ arylalkyl groups substituted with one or more bromo and optionally substituted with one or more fluoro. In some embodiments, R² is chosen from C₇₋₁₉ arylalkyl groups substituted with one or more bromo and substituted with one or more fluoro. In some embodiments, R² is chosen from C₇₋₁₉ arylalkyl groups substituted with one or more bromo and optionally substituted with one or more chloro. In some embodiments, R² is chosen from C₇₋₁₉ arylalkyl groups substituted with one or more bromo and substituted with one or more chloro. In some embodiments, R² is chosen from benzyl substituted with one or more bromo and optionally substituted with one or more fluoro. In some embodiments, R² is chosen from benzyl substituted with one or more bromo and substituted with one or more fluoro. In some embodiments, R² is chosen from benzyl substituted with one or more bromo and optionally substituted with one or more chloro. In some embodiments, R² is chosen from benzyl substituted with one or more bromo and substituted with one or more chloro.

In some embodiments, R² is chosen from C₇₋₁₉ arylalkyl groups substituted with one or more bromo and optionally substituted with one or more groups independently chosen from C₁₋₈ alkyl groups. In some embodiments, R² is chosen from C₇₋₁₉ arylalkyl groups substituted with one or more bromo and substituted with one or more groups independently chosen from C₁₋₈ alkyl groups. In some embodiments, R² is benzyl, wherein the benzyl is substituted with one or more bromo and optionally substituted with one or more groups independently chosen from C₁₋₈ alkyl groups. In some embodiments, R² is benzyl, wherein the benzyl is substituted with one or more bromo and substituted with one or more groups independently chosen from C₁₋₈ alkyl groups.

In some embodiments, R² is chosen from

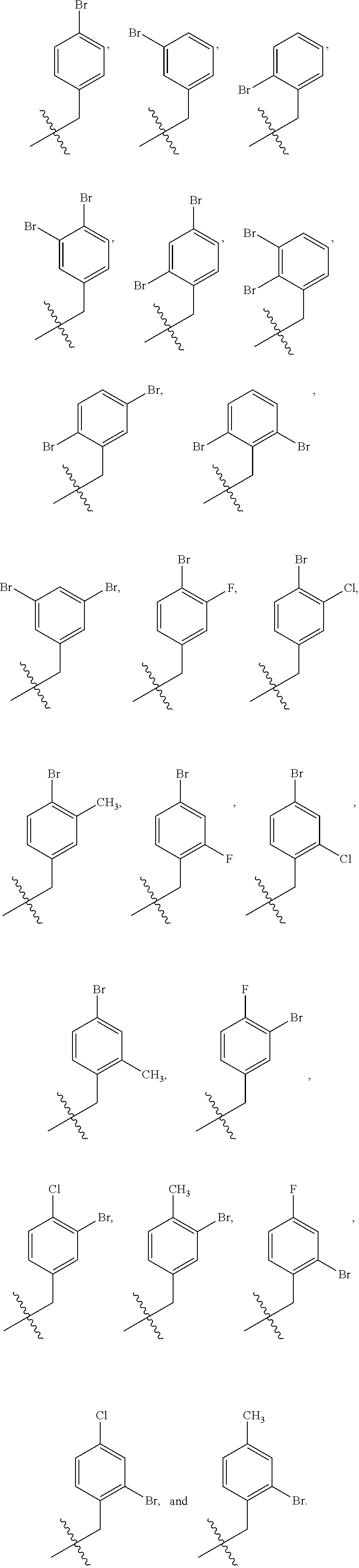

In some embodiments, R² is chosen from C₇₋₁₉ arylalkyl groups substituted with one or more bromo and optionally substituted with one or more groups independently chosen from C₁₋₈ hydroxyalkyl groups. In some embodiments, R² is chosen from C₇₋₁₉ arylalkyl groups substituted with one or more bromo and substituted with one or more groups independently chosen from C₁₋₈ hydroxyalkyl groups. In some embodiments, R² is benzyl, wherein the benzyl is substituted with one or more bromo and optionally substituted with one or more groups independently chosen from C₁₋₈ hydroxyalkyl groups. In some embodiments, R² is benzyl, wherein the benzyl is substituted with one or more bromo and substituted with one or more groups independently chosen from C₁₋₈ hydroxyalkyl groups.

In some embodiments, R² is chosen from C₇₋₁₉ arylalkyl groups substituted with one or more bromo and optionally substituted with one or more groups independently chosen from C₁₋₈ haloalkyl groups. In some embodiments, R² is chosen from C₇₋₁₉ arylalkyl groups substituted with one or more bromo and substituted with one or more groups independently chosen from C₁₋₈ haloalkyl groups. In some embodiments, R² is benzyl, wherein the benzyl is substituted with one or more bromo and optionally substituted with one or more groups independently chosen from C₁₋₈ haloalkyl groups. In some embodiments, R² is benzyl, wherein the benzyl is substituted with one or more bromo and substituted with one or more groups independently chosen from C₁₋₈ haloalkyl groups.

In some embodiments, R² is chosen from C₇₋₁₉ arylalkyl groups substituted with one or more bromo and optionally substituted with one or more groups independently chosen from C₆₋₁₈ aryl groups. In some embodiments, R² is chosen from C₇₋₁₉ arylalkyl groups substituted with one or more bromo and substituted with one or more groups independently chosen from C₆₋₁₈ aryl groups. In some embodiments, R² is benzyl, wherein the benzyl is substituted with one or more bromo and optionally substituted with one or more groups independently chosen from C₆₋₁₈ aryl groups. In some embodiments, R² is benzyl, wherein the benzyl is substituted with one or more bromo and substituted with one or more groups independently chosen from C₆₋₁₈ aryl groups.

In some embodiments, R² is chosen from C₇₋₁₉ arylalkyl groups substituted with one or more bromo and optionally substituted with one or more groups independently chosen from
—OZ³ groups. In some embodiments, R² is chosen from C₇₋₁₉ arylalkyl groups substituted with one or more bromo and substituted with one or more groups independently chosen from —OZ³ groups. In some embodiments, R² is benzyl, wherein the benzyl is substituted with one or more bromo and optionally substituted with one or more groups independently chosen from —OZ³ groups. In some embodiments, R² is benzyl, wherein the benzyl is substituted with one or more bromo and substituted with one or more groups independently chosen from —OZ³ groups.

In some embodiments, R² is chosen from C₇₋₁₉ arylalkyl groups substituted with one or more bromo and optionally substituted with one or more groups independently chosen from
—C(═O)OZ³ groups. In some embodiments, R² is chosen from C₇₋₁₉ arylalkyl groups substituted with one or more bromo and substituted with one or more groups independently chosen from
—C(═O)OZ¹ groups. In some embodiments, R² is benzyl, wherein the benzyl is substituted with one or more bromo and optionally substituted with one or more groups independently chosen from —C(═O)OZ³ groups. In some embodiments, R² is benzyl, wherein the benzyl is substituted with one or more bromo and substituted with one or more groups independently chosen from
—C(═O)OZ³ groups.

In some embodiments, R² is chosen from C₇₋₁₉ arylalkyl groups substituted with one or more bromo and optionally substituted with one or more groups independently chosen from
—C(═O)NZ³Z⁴ groups. In some embodiments, R² is chosen from C₇₋₁₉ arylalkyl groups substituted with one or more bromo and substituted with one or more groups independently chosen from —C(═O)NZ³Z⁴ groups. In some embodiments, R² is benzyl, wherein the benzyl is substituted with one or more bromo and optionally substituted with one or more groups independently chosen from —C(═O)NZ³Z⁴ groups. In some embodiments, R² is benzyl, wherein the benzyl is substituted with one or more bromo and substituted with one or more groups independently chosen from —C(═O)NZ³Z⁴ groups.

In some embodiments, R² is chosen from C₇₋₁₉ arylalkyl groups substituted with one or more bromo and optionally substituted with one or more groups independently chosen from
—SO₂Z³ groups. In some embodiments, R² is chosen from C₇₋₁₉ arylalkyl groups substituted with one or more bromo and substituted with one or more groups independently chosen from —SO₂Z³ groups. In some embodiments, R² is benzyl, wherein the benzyl is substituted with one or more bromo and optionally substituted with one or more groups independently chosen from —SO₂Z³ groups. In some embodiments, R² is benzyl, wherein the benzyl is substituted with one or more bromo and substituted with one or more groups independently chosen from —SO₂Z³ groups.

In some embodiments, R³ is chosen from C₁₋₁₃ heteroaryl groups optionally substituted with one or more groups independently chosen from R⁴, C₁₋₈ alkyl, C₁₋₈ haloalkyl, —C(═O)OZ⁵, and —C(═O)NZ⁵Z⁶ groups. In some embodiments, R³ is chosen from C₁₋₁₃ heteroaryl groups substituted with one or more groups independently chosen from R⁴, C₁₋₈ alkyl, C₁₋₈ haloalkyl, —C(═O)OZ⁵, and —C(═O)NZ⁵Z² groups. In some embodiments, R³ is chosen from C₂₋₆ heteroaryl groups. In some embodiments, R³ is chosen from C₂₋₄ heteroaryl groups.

In some embodiments, R³ is chosen from C₁₋₁₃ heteroaryl groups optionally substituted with one or more groups independently chosen from R⁴. In some embodiments, R³ is chosen from C₂₋₄ heteroaryl groups optionally substituted with one or more groups independently chosen from R⁴. In some embodiments, R³ is chosen from C₁₋₁₃ heteroaryl groups substituted with one or more groups independently chosen from R⁴. In some embodiments, R³ is chosen from C₂₋₄ heteroaryl groups substituted with one or more groups independently chosen from R⁴.

In some embodiments, R⁴ is chosen from C₆₋₁₈ aryl groups optionally substituted with one or more groups independently chosen from halo groups. In some embodiments, R⁴ is chosen from phenyl optionally substituted with one or more groups independently chosen from halo groups. In some embodiments, R⁴ is chosen from C₆₋₁₈ aryl groups substituted with one or more groups independently chosen from halo groups. In some embodiments, R⁴ is chosen from phenyl substituted with one or more groups independently chosen from halo groups. In some embodiments, at least one halo group is fluoro.

In some embodiments, R³ is chosen from

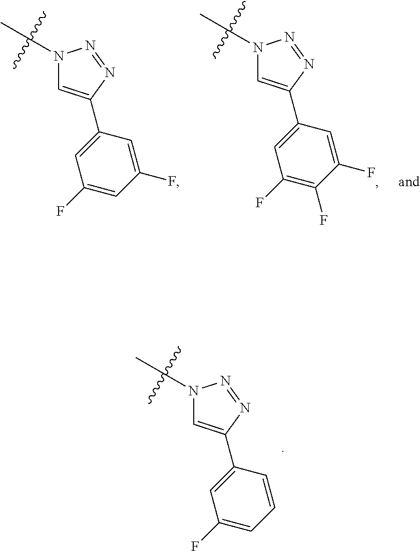 [SMILES: *n1cc(-c2cc(F)cc(F)c2)nn1 ; *n1cc(-c2cc(F)c(F)c(F)c2)nn1 ; *n1cc(-c2cccc(F)c2)nn1]

In some embodiments, R³ is

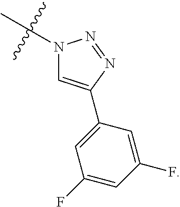 [SMILES: *n1cc(-c2cc(F)cc(F)c2)nn1]

In some embodiments, R³ is

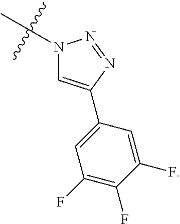 [SMILES: *n1cc(-c2cc(F)c(F)c(F)c2)nn1]

In some embodiments, R³ is

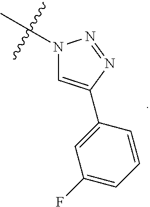 [SMILES: *n1cc(-c2cccc(F)c2)nn1]

In some embodiments, X is chosen from —C—, —O—, —S—, and —N(R⁵)—, wherein R⁵ is chosen from H, C₁₋₈ alkyl, and C₁₋₈ haloalkyl groups. In some embodiments, X is —C—. In some embodiments, X is —O—. In some embodiments, X is —S—. In some embodiments, X is —N(R⁵)—. In some embodiments, R⁵ is chosen from H, C₁₋₁₄ alkyl, and C₁₋₄ haloalkyl groups. In some embodiments, R⁵ is H. In some embodiments, R⁵ is chosen from C₁₋₄ alkyl groups.

In some embodiments, Y is H. In some embodiments, Y is chosen from halo groups. In some embodiments, Y is fluoro. In some embodiments, Y is chosen from —OZ⁹ groups. In some embodiments, Z⁹ is chosen from H and C₁₋₄ alkyl groups. In some embodiments, Z⁹ is chosen from H and C₁₋₄ haloalkyl groups. In some embodiments, Z⁹ is chosen from H and C₁₋₄ deuteroalkyl groups. In some embodiments, Y is —OH. In some embodiments, Y is —OMe. In some embodiments, Y is —OEt. In some embodiments, Y is -OnPr. In some embodiments, Y is —OiPr. In some embodiments, Y is —OCHD₂. In some embodiments, Y is —OCD₃. In some embodiments, Y is —OCHDCH₂D. In some embodiments, Y is —CH₂CD₃. In some embodiments, Y is —OCD₂CD₃. In some embodiments, Y is —OCD₂CD₂CD₃. In some embodiments, Y is —OCD(CD₃)₂. In some embodiments, Y is —OCF₃. In some embodiments, Y is —OCHF₂. In some embodiments, Y is —OCH₂F. In some embodiments, Y is —OCF₂CF₃.

In some embodiments, each of Z¹, Z², Z³, Z⁴, Z⁵, Z⁶, Z⁷, Z⁸, Z⁹, T¹, and T² is unsubstituted. In some embodiments, at least one of Z¹, Z², Z³, Z⁴, Z⁵, Z⁶, Z⁷, Z⁸, Z⁹, T¹, and T² is substituted. In some embodiments, at least one of Z¹, Z², Z³, Z⁴, Z⁵, Z⁶, Z⁷, Z⁸, Z⁹, T¹, and T² is substituted with one or more groups independently chosen from halo and —OR⁶ groups. In some embodiments, at least one of Z¹, Z², Z³, Z⁴, Z⁵, Z⁶, Z⁷, Z⁸, Z⁹, T¹, and T² is substituted with one or more groups independently chosen from halo groups. In some embodiments, at least one of Z¹, Z², Z³, Z, Z Z⁶, Z, Z⁸, Z⁹, T¹, and T² is substituted with one or more groups independently chosen from —OR⁶ groups.

In some embodiments, each of Z¹, Z², Z³, Z⁴, Z⁵, Z⁶, Z⁷, Z⁸, and Z⁹ is unsubstituted. In some embodiments, at least one of Z¹, Z², Z³, Z⁴, Z⁵, Z⁶, Z⁷, Z⁸, and Z⁹ is substituted. In some embodiments, at least one of Z¹, Z², Z³, Z⁴, Z⁵, Z⁶, Z⁷, Z⁸, and Z⁹ is substituted with one or more groups independently chosen from halo and —OR⁶ groups. In some embodiments, at least one of Z¹, Z², Z³, Z⁴, Z⁵, Z⁶, Z⁷, Z⁸, and Z⁹ is substituted with one or more groups independently chosen from halo groups. In some embodiments, at least one of Z¹, Z², Z³, Z⁴, Z⁵, Z⁶, Z⁷, Z⁸, and Z⁹ is substituted with one or more groups independently chosen from —OR⁶ groups. In some embodiments, R⁶ is H. In some embodiments, R⁶ is independently chosen from C₁₋₈ alkyl groups. In some embodiments, R⁶ is independently chosen from C₁₋₄ alkyl groups. In some embodiments, R⁶ is independently chosen from methyl, ethyl, propyl, and butyl groups. In some embodiments, the halo group is fluoro.

In some embodiments, the compound of Formula (I) is chosen from:

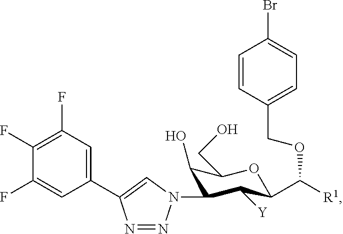

prodrugs thereof, and pharmaceutically acceptable salts of any of the foregoing.

In some embodiments, the compound of Formula (I) is chosen from:

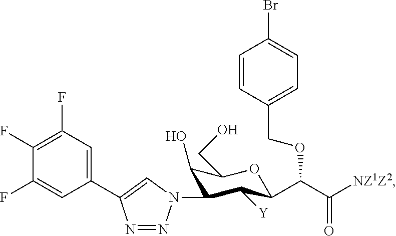

prodrugs thereof, and pharmaceutically acceptable salts of any of the foregoing.

In some embodiments, the compound of Formula (I) is chosen from:

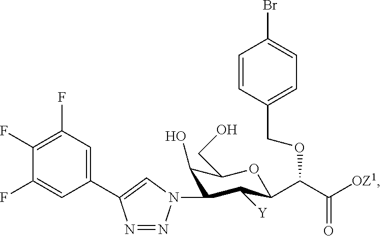

prodrugs thereof, and pharmaceutically acceptable salts of any of the foregoing.

In some embodiments, the compound of Formula (I) is chosen from:

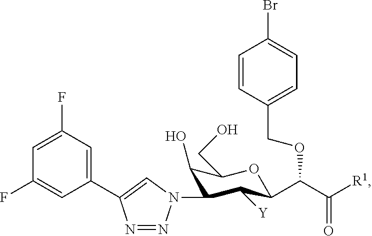

prodrugs thereof, and pharmaceutically acceptable salts of any of the foregoing.

In some embodiments, the compound of Formula (I) is chosen from:

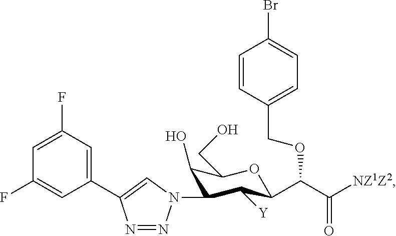

prodrugs thereof, and pharmaceutically acceptable salts of any of the foregoing.

In some embodiments, the compound of Formula (I) is chosen from:

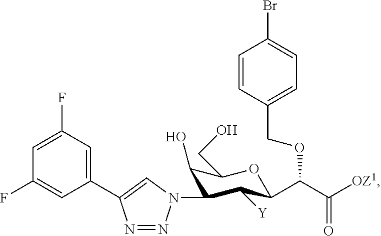

prodrugs thereof, and pharmaceutically acceptable salts of any of the foregoing.

In some embodiments, the compound of Formula (I) is chosen from:

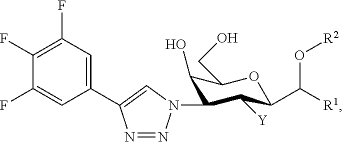

prodrugs thereof, and pharmaceutically acceptable salts of any of the foregoing, wherein
R¹ is chosen from

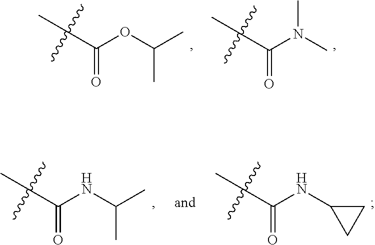

R² is chosen from

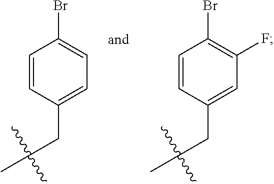 [SMILES: *Cc1ccc(Br)cc1 ; *Cc1ccc(Br)c(F)c1]

and
Y is chosen from —OMe and —OCD₃.

In some embodiments, the compound of Formula (I) is chosen from:

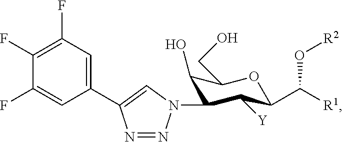

prodrugs thereof, and pharmaceutically acceptable salts of any of the foregoing, wherein
R¹ is chosen from

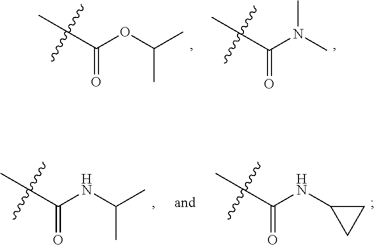

R¹ is chosen from

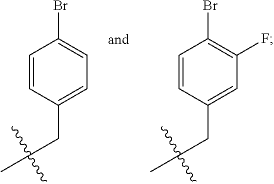 [SMILES: *Cc1ccc(Br)cc1 ; *Cc1ccc(Br)c(F)c1]

and
Y is chosen from —OMe and —OCD₃.

In some embodiments, the compound of Formula (I) is chosen from:

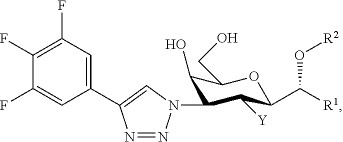

wherein:
R¹ is chosen from

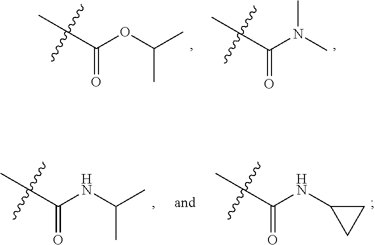

R² is chosen from

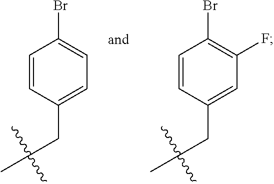 [SMILES: *Cc1ccc(Br)cc1 ; *Cc1ccc(Br)c(F)c1]

and
Y is chosen from —OMe and —OCD₃.

Also provided are pharmaceutical compositions comprising at least one compound of Formula (I). Such pharmaceutical compositions are described in greater detail herein. These compounds and compositions may be used in the methods described herein.

In some embodiments, a method for treating and/or preventing at least one disease, disorder, and/or condition where inhibition of galectin-3 mediated functions may be useful is disclosed, the method comprising administering to a subject in need thereof at least one compound of Formula (I) and/or a pharmaceutical composition comprising at least one compound of Formula (I).

In some embodiments, a method for treating and/or preventing at least one inflammatory disease, disorder, and/or condition in which the adhesion and/or migration of cells occurs in the disease, disorder, and/or condition is disclosed, the method comprising administering to a subject in need thereof at least one compound of Formula (I) and/or a pharmaceutical composition comprising at least one compound of Formula (I).

In some embodiments, a method for regulating the diffusion, compartmentalization, and/or endocytosis of plasma membrane glycoproteins and/or glycolipids is disclosed, the method comprising administering to a subject in need thereof at least one compound of Formula (I) and/or a pharmaceutical composition comprising at least one compound of Formula (I).

In some embodiments, a method for regulating the selection, activation, and/or arrest of T cells, receptor kinase signaling, and/or the functionality of membrane receptors is disclosed, the method comprising administering to a subject in need thereof at least one compound of Formula (I) and/or a pharmaceutical composition comprising at least one compound of Formula (I).

In some embodiments, a method for treating and/or preventing at least one fibrosis is disclosed, the method comprising administering to a subject in need thereof at least one compound of Formula (I) and/or a pharmaceutical composition comprising at least one compound of Formula (I). In some embodiments, the at least one compound of Formula (I) inhibits lattice formation between galectin-3 and glycosylated ligands.

In some embodiments, a method for treating and/or preventing a cancer is disclosed, the method comprising administering to a subject in need thereof an effective amount of at least one compound of Formula (I) and/or a pharmaceutical composition comprising at least one compound of Formula (I). In some embodiments, the at least one compound of Formula (I) and/or pharmaceutical composition comprising the at least one compound of Formula (I) may be administered in conjunction with (i.e., as an adjunct therapy, which is also called adjunctive therapy) chemotherapy and/or radiotherapy.

The chemotherapy and/or radiotherapy may be referred to as the primary anti-tumor or anti-cancer therapy that is being administered to the subject to treat the particular cancer. In some embodiments, a method for reducing (i.e., inhibiting, diminishing) chemosensitivity and/or radiosensitivity of hematopoietic stem cells (HSC) to the chemotherapeutic drug(s) and/or radiotherapy, respectively, is disclosed, the method comprising administering to a subject in need thereof an effective amount of at least one compound of Formula (I) and/or a pharmaceutical composition comprising at least one compound of Formula (I).

In some embodiments, a method for enhancing (i.e., promoting) survival of hematopoietic stem cells is provided, the method comprising administering to a subject in need thereof at least one compound of Formula (I) and/or a pharmaceutical composition comprising at least one compound of Formula (I).

In some embodiments, a method for decreasing the likelihood of occurrence of metastasis of cancer cells (also called tumor cells herein) is disclosed, the method comprising administering to a subject in need thereof an effective amount of at least one compound of Formula (I) and/or a pharmaceutical composition comprising at least one compound of Formula (I).

In some embodiments, a method for treating and/or preventing at least one cancer in which the cancer cells may leave the primary site is disclosed, the method comprising administering to a subject in need thereof an effective amount of at least one compound of Formula (I) and/or a pharmaceutical composition comprising at least one compound of Formula (I). A primary site may be, for example, solid tissue (e.g., breast or prostate) or the bloodstream.

In some embodiments, a method for treating and/or preventing at least one cancer in which it is desirable to mobilize cancer cells from a site into the bloodstream and/or retain the cancer cells in the bloodstream is disclosed, the method comprising administering to a subject in need thereof an effective amount of at least one compound of Formula (T) and/or a pharmaceutical composition comprising at least one compound of Formula (I).

In some embodiments, a method for decreasing the likelihood of occurrence of infiltration of cancer cells into bone marrow is disclosed, the method comprises administering to a subject in need thereof an effective amount of at least one compound of Formula (I) and/or a pharmaceutical composition comprising at least one compound of Formula (I).

In some embodiments, a method for releasing cells into circulating blood and enhancing retention of the cells in the blood is disclosed, the method comprising administering to a subject in need thereof an effective amount of at least one compound of Formula (I) and/or a pharmaceutical composition comprising at least one compound of Formula (I). In some embodiments, the method further includes collecting the released cells. In some embodiments, collecting the released cells utilizes apheresis. In some embodiments, the released cells are stem cells (e.g., bone marrow progenitor cells). In some embodiments, G-CSF is administered to the individual.

In some embodiments, a method for treating and/or preventing checkpoint inhibition of T-cells is disclosed, the method comprising administering to a subject in need thereof an effective amount of at least one compound of Formula (I) and/or a pharmaceutical composition comprising at least one compound of Formula (I).

In some embodiments, a method for treating and/or preventing thrombosis is disclosed, the method comprising administering to a subject in need thereof an effective amount of at least one compound of Formula (I) and/or a pharmaceutical composition comprising at least one compound of Formula (I).

In some embodiments, a method for treating and/or preventing one cardiovascular disease, disorder, and/or condition is disclosed, the method comprising administering to a subject in need thereof an effective amount of at least one compound of Formula (I) and/or a pharmaceutical composition comprising at least one compound of Formula (I).

In some embodiments, a method for treating and/or preventing atherosclerosis is disclosed, the method comprising administering to a subject in need thereof an effective amount of at least one compound of Formula (I) and/or a pharmaceutical composition comprising at least one compound of Formula (I).

In some embodiments, a method for inhibiting the rejection of transplanted tissue is disclosed, the method comprising administering to a subject in need thereof an effective amount of at least one compound of Formula (I) and/or a pharmaceutical composition comprising at least one compound of Formula (I).

In some embodiments, a method for treating and/or preventing pathological angiogenesis is disclosed, the method comprising administering to a subject in need thereof an effective amount of at least one compound of Formula (I) and/or a pharmaceutical composition comprising at least one compound of Formula (I).

In some embodiments, a method for treating and/or preventing an epileptic syndrome is disclosed, the method comprising administering to a subject in need thereof at least one compound of Formula (I) and/or a pharmaceutical composition comprising at least one compound of Formula (I).

In some embodiments, a method for treating and/or preventing a neurodegenerative disease is disclosed, the method comprising administering to a subject in need thereof an effective amount of at least one compound of Formula (I) and/or a pharmaceutical composition comprising at least one compound of Formula (I).

In some embodiments, a method for treating and/or preventing α-synucleinopathies is disclosed, the method comprising administering to a subject in need thereof an effective amount of at least one compound of Formula (I) and/or a pharmaceutical composition comprising at least one compound of Formula (I).

In some embodiments, a method for treating and/or preventing a fibrosing disease or condition is disclosed, the method comprising administering to a subject in need thereof an effective amount of at least one compound of Formula (I) and/or a pharmaceutical composition comprising at least one compound of Formula (I).

In some embodiments, a method for treating and/or preventing sinusoidal obstruction syndrome (SOS) or complications associated therewith is disclosed, the method comprising administering to a subject in need thereof an effective amount of at least one compound of Formula (I) and/or a pharmaceutical composition comprising at least one compound of Formula (I).

In some embodiments, a compound of Formula (I) and/or a pharmaceutical composition comprising at least one compound of Formula (I) may be used for the preparation and/or manufacture of a medicament for use in treating and/or preventing at least one of the diseases, disorders, and/or conditions described herein.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example, “n is an integer ranging from 0 to 2” includes, independently, 0, 1, and 2.

The terms “at least one” and “one or more” are intended to be synonymous and to refer to no less than one but possibly more, such as one, two, three, etc. For example, the term “at least one C₁₋₄ alkyl group” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic, or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The terms “galectin-3 antagonist,” “galectin-3 inhibitor,” and “inhibitors of galectin-3” are used interchangeably herein, and include inhibitors of galectin-3 only, as well as inhibitors of galectin-3 and one or more other galectin, such as galectin-1, galectin-2, galectin-4, galectin-5, galectin-6, galectin-7, galectin-8, galectin-9, galectin-10, galectin-11, and galectin-12.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “deuteroalkyl” includes alkyl groups, as defined herein, substituted by at least one deuterium (also known as hydrogen-2 or ²H or represented by the chemical symbol D). Non-limiting examples of deuteroalkyl groups include —CHD₂, —CD₃, —CHDCH₂D, —CH₂CD₃, —CD₂CD₃, —CD₂CD₂CD₃, and —CD(CD₃)₂. Unless stated otherwise specifically in the specification, a deuteroalkyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

Unless stated otherwise specifically in the specification, substituents may be optionally substituted.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible geometric isomers, e.g., Z and E isomers (cis and trains isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof.

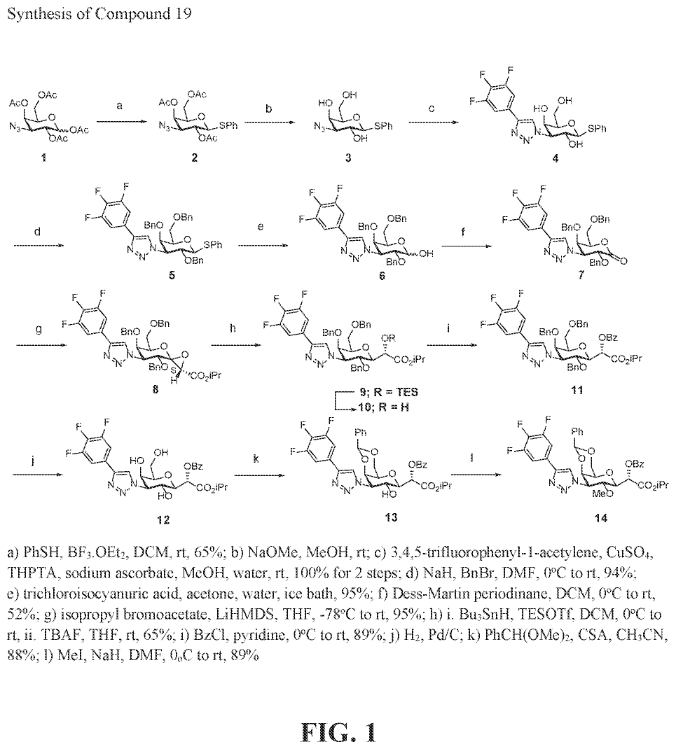

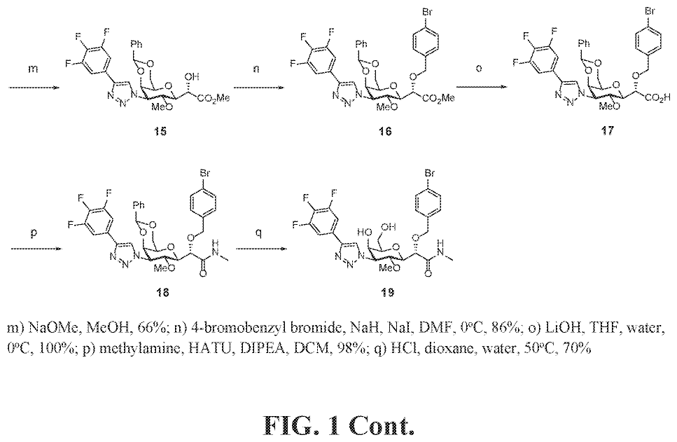

Compounds of Formula (I) may be prepared as shown in FIGS. 1 through 4 and described in WO2020/139960. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the synthesis as needed. In general, starting components may be obtained from sources such as Sigma Aldrich, Alfa Aesar, Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl,
t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to —C(O)R″ (wherein R″ is alkyl, aryl, or arylalkyl), p-methoxybenzyl, trityl, and the like. Suitable protecting groups for carboxylic acid include but are not limited to alkyl, aryl, or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatises that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell-based activity assays.

An inhibition assay may be used to screen for antagonists of galectin-3. For example, an assay may be performed to characterize the capability of a compound described herein to inhibit interaction of galectin-3 with a Galβ1-3GlcNAc carbohydrate structure. The inhibition assay may be a competitive binding assay, which allows the determination of IC₅₀ values. By way of example, a Galβ1-3GlcNAc polymer may be immobilized onto a matrix; a composition may be added to reduce nonspecific binding; the immobilized Galβ1-3GlcNAc polymer may be contacted with the candidate compound in the presence of galectin-3 group under conditions and for a time sufficient to permit galectin-3 to bind to the immobilized Galβ1-3GlcNAc polymer; the immobilized Galβ1-3GlcNAc polymer may be washed; and the amount of galectin-3 bound to the immobilized Galβ1-3GlcNAc polymer may be detected. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatiable cell lines, transformed cell lines, and the like.

As described herein, methods for characterizing galectin-3 antagonists include animal model studies. Non-limiting examples of animal models for liquid cancers used in the art include multiple myeloma (see, e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 Apr. 1: 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

The compounds of the present disclosure and the pharmaceutical compositions comprising at least one of such compounds may be useful in methods for treating and/or preventing a disease or disorder that is treatable by inhibiting at least one activity of galectin-3 (and/or inhibiting binding of galectin-3 to ligand(s), which in turn inhibits a biological activity).

The compounds of the present disclosure and pharmaceutical compositions comprising at least one such compound may be useful in methods for treating and/or preventing at least one inflammatory disease. Inflammation comprises reaction of vascularized living tissue to injury. By way of example, although galectin-3 mediated cell adhesion may be important to the body's anti-infective immune response, in other circumstances, galectin-3 mediated cell adhesion may be undesirable or excessive, resulting in tissue damage and/or scarring instead of repair. For example, many pathologies (such as autoimmune and inflammatory diseases, shock and reperfusion injuries) involve abnormal adhesion of white blood cells. Therefore, inflammation affects blood vessels and adjacent tissues in response to an injury or abnormal stimulation by a physical, chemical, or biological agent. Examples of inflammatory diseases, disorders, or conditions include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the compounds described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischemic episodes, prevention of restenosis associated with vascular stenting, and for undesirable angiogenesis, for example, angiogenesis associated with tumor growth.

As discussed in detail herein, a disease or disorder to be treated or prevented is a cancer and related metastasis and includes cancers that comprise solid tumor(s) and cancers that comprise liquid tumor(s). The compounds of the present disclosure and pharmaceutical compositions comprising at least one such compound may be useful in methods for preventing and/or treating cancer. In some embodiments, the at least one compound may be used for treating and/or preventing metastasis and/or for inhibiting (slowing, retarding, or preventing) metastasis of cancer cells.

In some embodiments, at least one compound of Formula (I) and/or a pharmaceutical composition comprising at least one compound of Formula (I) is administered to a cancer patient in remission. In some embodiments, the at least one compound of Formula (I) and/or a pharmaceutical composition comprising at least one compound of Formula (I) is administered as a cancer vaccine to stimulate marrow infiltrating lymphocytes (“MILs”) in a cancer patient or cancer survivor to prevent relapse.

In some embodiments, a method of treating cancer and/or preventing a cancer relapse is disclosed, wherein the method comprises administering to a patient in need thereof an effective amount of at least one compound of Formula (I) and/or a pharmaceutical composition comprising at least one compound of Formula (I), wherein the amount of the at least one compound of Formula (I) administered is sufficient to mobilize MILs of the patient into the peripheral blood.

In some embodiments, a method of treating cancer and/or preventing a cancer relapse is provided comprising administering to a donor patient at least one compound of Formula (I) and/or a pharmaceutical composition comprising at least one compound of Formula (I) in an amount of sufficient to mobilize MILs of the patient out of the marrow (e.g., into the peripheral blood), recovering MILS (e.g., recovering them from the peripheral blood), and transplanting at least a portion of the MIL cell population to the donor patient or another patient. In some embodiments, the MIL cell population is expanded ex vivo before transplantation.

In some embodiments, a method of preventing cancer is provided comprising administering to a donor patient at least one compound of Formula (I) and/or a pharmaceutical composition comprising at least one compound of Formula (I) in an amount sufficient to mobilize MILs of the patient out of the bone marrow (e.g., into the peripheral blood), recovering MILs (e.g., recovering them from the peripheral blood), and transplanting at least a portion of MIL cell population to a subject (e.g., a non-cancer patient, a patient suffering from a different form or type of cancer than the donor patient, etc.). In some embodiments, the MIL cell population is expanded ex vivo before transplantation.

In some embodiments, the compounds of present disclosure and pharmaceutical compositions comprising at least one such compound may be used for decreasing (i.e., reducing) the likelihood of occurrence of metastasis of cancer cells in an individual (i.e., subject, patient) who is in need thereof. The compounds of the present disclosure and compositions comprising at least one such compound may be used for decreasing (i.e., reducing) the likelihood of occurrence of infiltration of cancer cells into bone marrow in an individual who is in need thereof. The individuals (or subjects) in need of such treatments include subjects who have been diagnosed with a cancer, which includes cancers that comprise solid tumor(s) and cancers that comprise liquid tumor(s).

Non-limiting examples of cancers include colorectal cancers, liver cancers, gastric cancers, lung cancers, brain cancers, kidney cancers, bladder cancers, thyroid cancers, prostate cancers, ovarian cancers, cervical cancers, uterine cancers, endometrial cancers, melanomas, breast cancers, and pancreatic cancers. Liquid tumors can occur in the blood, bone marrow, the soft, sponge-like tissue in the center of most bones, and lymph nodes and include leukemias (e.g., AML, ALL, CLL, and CML), lymphomas, and myelomas (e.g., multiple myeloma). Lymphomas include Hodgkin lymphoma, which is marked by the presence of a type of cell called the Reed-Sternberg cell, and non-Hodgkin lymphomas, which includes a large, diverse group of cancers of immune system cells. Non-Hodgkin lymphomas can be further divided into cancers that have an indolent (slow-growing) course and those that have an aggressive (fast-growing) course, and which subtypes respond to treatment differently.

The compounds of the present disclosure and pharmaceutical compositions comprising at least one such compound may be administered as an adjunct therapy to chemotherapy and/or radiotherapy, which is/are being delivered to the subject as primary therapy for treating the cancer. The chemotherapy and/or radiotherapy that may be administered depend upon several factors including the type of cancer, location of the tumor(s), stage of the cancer, age and gender and general health status of the subject. A person of ordinary skill in the medical art can readily determine the appropriate chemotherapy regimen and/or radiotherapy regimen for the subject in need. The person of ordinary skill in the medical art can also determine, with the aid of preclinical and clinical studies, when the compound of the present disclosure or pharmaceutical composition comprising at least one such compound should be administered to the subject, that is whether the compound or composition is administered prior to, concurrent with, or subsequent to a cycle of the primary chemotherapy or radiation treatment.

In some embodiments, a method for inhibiting activation of hepatic and/or pancreatic stellate cells is disclosed, the method comprising administering at least one compound of Formula (I) and/or a pharmaceutical composition comprising at least one compound of Formula (I).

In some embodiments, a method for inhibiting adhesion of metastasized tumor cells is disclosed, the method comprising administering at least one compound of Formula (T) and/or a pharmaceutical composition comprising at least one compound of Formula (I).

In some embodiments, a method for inhibiting cell-cell interactions and/or interactions between cells and the extracellular matrix where the cell-cell interactions and cell-matrix are induced by galectin-3 molecules bound carbohydrates found on the surface of cells is disclosed, the method comprising administering at least one compound of Formula (I) and/or a pharmaceutical composition comprising at least one compound of Formula (I). In some embodiments, the cells are tumor cells and cell-cell interactions and/or cell-matrix are responsible for the development of at least one tumor disease.

In some embodiments, a method for reducing the rate of growth of tumor cells which express galectin-3 is disclosed, the method comprising administering at least one compound of Formula (I) and/or a pharmaceutical composition comprising at least one compound of Formula (I). In some embodiments, the level of at least one G1/S cyclin in the tumor cell is reduced.

As described herein, at least one of the compounds of the present disclosure or a pharmaceutical composition comprising at least one such compound may be administered in combination with at least one additional anti-cancer agent. Chemotherapy may comprise one or more chemotherapeutic agents. For example, chemotherapy agents, radiotherapeutic agents, inhibitors of phosphoinositide-3 kinase (PI3K), and inhibitors of VEGF may be used in combination with a compound of Formula (I) described herein. Non-limiting examples of inhibitors of PI3K include the compound named by Exelixis as “XL499.” Non-limiting examples of VEGF inhibitors include the compound called “cabo” (previously known as XL184). Many other chemotherapeutics are small organic molecules. As understood by a person of ordinary skill in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents; antimetabolites; anthracyclines, plant alkaloids; and topoisomerase inhibitors.

The compounds of the present disclosure or pharmaceutical compositions comprising at least one such compound may function independently from the anti-cancer agent or may function in coordination with the anti-cancer agent, e.g., by enhancing effectiveness of the anti-cancer agent or vice versa. Accordingly, provided herein are methods for enhancing (i.e., enhancing, promoting, improving the likelihood of, enhancing in a statistically or biologically significant manner) and/or maintaining survival of hematopoietic stem cells (HSC) in a subject who is treated with and/or will be treated with a chemotherapeutic drug(s) and/or radioactive therapy, respectively, comprising administering at least one compound of Formula (I) as described herein. In some embodiments, the subject receives and/or will receive both chemotherapy and radiation therapy. Also, provided herein is a method for reducing (i.e., reducing, inhibiting, diminishing in a statistically or biologically significant manner) chemosensitivity and/or radiosensitivity of hematopoietic stem cells (HSC) to the chemotherapeutic drug(s) and/or radioactive therapy, respectively, in a subject in need thereof. Because repeated cycles of chemotherapy and radiotherapy often diminish the ability of HSCs to recover and replenish bone marrow, the glycomimetic compounds described herein may be useful for subjects who will receive more than one cycle, such as at least two, three, four or more cycles, of chemotherapy and/or radiotherapy. HSCs reside in the bone marrow and generate the cells that are needed to replenish the immune system and the blood. Anatomically, bone marrow comprises a vascular niche that is adjacent to bone endothelial sinuses (see, e.g., Kiel et al., Cell 121:1109-21 (2005); Sugiyama et al., Immunity 25:977-88 (2006); Mendez-Ferrer et al., Nature 466:829-34 (2010); Butler et al., Cell Stem Cell 6:251-64 (2010)). Additionally, galectin-3 has recently been reported to interfere with hematopoiesis and promote terminal differentiation of myeloid progenitors (see, e.g., Brand et al., Cell Tissue Res 346:427-37 (2011)).

In addition, the administration of at least one compound of the present disclosure or pharmaceutical composition comprising at least one such compound may be in conjunction with one or more other therapies, e.g., for reducing toxicities of therapy. For example, at least one palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one compound described herein may be administered before, after, or concurrently with administration of at least one additional anti-cancer agent or at least one palliative agent to reduce a side effect of therapy. When administration is concurrent, the combination may be administered from a single container or two (or more) separate containers.

Cancer cells (also called herein tumor cells) that may be prevented (i.e., inhibited, slowed) from metastasizing, from adhering to an endothelial cell, or from infiltrating bone marrow include cells of solid tumors and liquid tumors (including hematological malignancies). Examples of solid tumors are described herein and include, but are not limited to, colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, and lymph nodes and include leukemia (e.g., AML, ALL, CLL, and CML), lymphoma (e.g., Hodgkin lymphoma and non-Hodgkin lymphoma), and myeloma (e.g., multiple myeloma). As used herein, the term cancer cells include mature, progenitor, and cancer stem cells.

Bones are a common location for cancer to infiltrate once leaving the primary tumor location. Once cancer resides in bone, it is frequently a cause of pain to the individual. In addition, if the particular bone affected is a source for production of blood cells in the bone marrow, the individual may develop a variety of blood cell related disorders. Breast and prostate cancer are examples of solid tumors that migrate to bones. Acute myelogenous leukemia (AML) and multiple myeloma (MM) are examples of liquid tumors that migrate to bones. Cancer cells that migrate to bone will typically migrate to the endosteal region of the bone marrow. Once cancer cells have infiltrated into the marrow, the cells become quiescent and are protected from chemotherapy. The compounds of the present disclosure may block infiltration of disseminated cancer cells into bone marrow. A variety of subjects may benefit from treatment with the compounds. Examples of such subjects include individuals with a cancer type having a propensity to migrate to bone where the tumor is still localized or the tumor is disseminated but not yet infiltrated bone, or where individuals with such a cancer type are in remission.

The cancer patient population most likely to respond to treatment using antagonists of galectin-3 (e.g., compounds of Formula (I)) described herein can be identified based on the mechanisms of action of galectin-3. For example, patients may be identified for treatment based on levels of galectin-3 detected in serum or plasma by a diagnostic assay such as the Abbott Laboratories ARCHITECT Galectin-3 assay, which can be used for determining galectin-3 in serum or plasma to stratify heart failure patients for proper treatment.

The compounds of the present disclosure and pharmaceutical compositions comprising at least one such compound may be useful in methods for mobilizing cells from the bone marrow to the peripheral vasculature and tissues. As discussed herein, in some embodiments, the compounds and compositions are useful for mobilizing hematopoietic cells, including hematopoietic stem cells and hematopoietic progenitor cells. In some embodiments, the compounds act as mobilizing agents of normal blood cell types. In some embodiments, the agents are used in methods for mobilizing mature white blood cells (which may also be called leukocytes herein), such as granulocytes (e.g., neutrophils, eosinophils, basophils), lymphocytes, and monocytes from the bone marrow or other immune cell compartments such as the spleen and liver. Methods are also provided for using the compounds of the present disclosure and pharmaceutical compositions comprising at least one such compound in methods for mobilizing tumor cells from the bone marrow. The tumor cells may be malignant cells (e.g., tumor cells that are metastatic cancer cells, or highly invasive tumor cells) in cancers. These tumor cells may be of hematopoietic origin or may be malignant cells of another origin residing in the bone.

In some embodiments, the methods using the compounds described herein are useful for mobilizing hematopoietic cells, such as hematopoietic stem cells and progenitor cells and leukocytes (including granulocytes such as neutrophils), which are collected (i.e., harvested, obtained) from the subject receiving a compound of Formula (I) and at a later time are administered back into the same subject (autologous donor) or administered to a different subject (allogeneic donor). Hematopoietic stem cell replacement and hematopoietic stem cell transplantation have been successfully used for treating a number of diseases (including cancers) as described herein and in the art. By way of example, stem cell replacement therapy or transplantation follows myeloablation of a subject, such as occurs with administration of high dose chemotherapy and/or radiotherapy. Desirably, an allogeneic donor shares sufficient HLA antigens with the recipient/subject to minimize the risk of host versus graft disease in the recipient (i.e., the subject receiving the hematopoietic stem cell transplant). Obtaining the hematopoietic cells from the donor subject (autologous or allogeneic) is performed by apheresis or leukapheresis. HLA typing of a potential donor and the recipient and apheresis or leukapheresis are methods routinely practiced in the clinical art.

By way of non-limiting example, autologous or allogenic hematopoietic stem cells and progenitors cells may be used for treating a recipient subject who has certain cancers, such as Hodgkin lymphoma, non-Hodgkin lymphoma, or multiple myeloma. Allogeneic hematopoietic stem cells and progenitors cells may be used, for example, for treating a recipient subject who has acute leukemia (e.g., AML, ALL); chronic lymphocytic leukemia (CLL): amegakaryocytosis/congenital thrombocytopenia, aplastic anemia/refractory anemia: familial erythrophagocytic lymphohistiocytosis; myelodysplastic syndrome/other myelodysplastic disorders; osteopetrosis; paroxysmal nocturnal hemoglobinuria; and Wiskott-Aldrich syndrome, for example. Exemplary uses for autologous hematopoietic stem cells and progenitors cells include treating a recipient subject who has amyloidosis; germ cell tumors (e.g., testicular cancer): or a solid tumor. Allogeneic hematopoietic stem cell transplants have also been investigated for use in treating solid tumors (see, e.g., Ueno et al., Blood 102:3829-36 (2003)).

In some embodiments of the methods described herein, the subject is not a donor of peripheral hematopoietic cells but has a disease, disorder, or condition for which mobilization of hematopoietic cells in the subject will provide clinical benefit. Stated another way, while this clinical situation is similar to autologous hematopoietic cell replacement, the mobilized hematopoietic cells are not removed and given back to the same subject at a later time as occurs, for example, with a subject who receives myeloablation therapy. Accordingly, methods are provided for mobilizing hematopoietic cells, such as hematopoietic stem cells and progenitor cells and leukocytes (including granulocytes, such as neutrophils), by administering at least once compound of Formula (I). Mobilizing hematopoietic stem cells and progenitor cells may be useful for treating an inflammatory condition or for tissue repair or wound healing. See, e.g., Mimeault et al., Clin. Pharmacol. Therapeutics 82:252-64 (2007).

In some embodiments, the methods described herein are useful for mobilizing hematopoietic leukocytes (white blood cells) in a subject, which methods may be used in treating diseases, disorders, and conditions for which an increase in white blood cells, such as neutrophils, eosinophils, lymphocytes, monocytes, basophils, will provide clinical benefit. For example, for cancer patients, the compounds of Formula (I) are beneficial for stimulating neutrophil production to compensate for hematopoietic deficits resulting from chemotherapy or radiation therapy. Other diseases, disorders, and conditions to be treated include infectious diseases and related conditions, such as sepsis. When the subject to whom at least one compound of Formula (I) is administered is a donor, neutrophils may be collected for administration to a recipient subject who has reduced hematopoietic function, reduced immune function, reduced neutrophil count, reduced neutrophil mobilization, severe chronic neutropenia, leucopenia, thrombocytopenia, anemia, and acquired immune deficiency syndrome. Mobilization of mature white blood cells may be useful in subjects to improve or to enhance tissue repair, and to minimize or prevent vascular injury and tissue damage, for example following liver transplantation, myocardial infarction or limb ischemia. See, e.g., Pelus, Curr. Opin. Hematol. 15:285-92 (2008); Lemoli et al., Haematologica 93:321-24 (2008).

The compounds of Formula (I) may be used in combination with one or more other agents that mobilize hematopoietic cells. Such agents include, for example, G-CSF; AMD3100 or other CXCR4 antagonists; GRO-β (CXCL2) and an N-terminal 4-amino truncated form (SB-251353); IL-8SDF-1α peptide analogs, CTCE-0021 and CTCE-0214; and the SDF1 analog, Met-SDF-1β (see, e.g., Pelus, supra and references cited therein). In some embodiments, a compound of Formula (I) may be administered with other mobilizing agents used in the art, which may permit administration of a lower dose of GCSF or AMD3100, for example, than required in the absence of a compound of Formula (I). The appropriate therapeutic regimen for administering a compound of Formula (I) in combination with another mobilizing agent or agents can be readily determined by a person skilled in the clinical art.

In some embodiments, the method further comprises administering a therapeutically effective amount of at least one MMP inhibitor, inflammatory cytokine inhibitor, mast cell inhibitor, NSAID, NO inhibitor, or antimicrobial compound.

In some embodiments, the method further comprises administering a therapeutically effective amount of velafermin and/or palifermin.

In some embodiments, the method further comprises administering a therapeutically effective amount of Davanat®, mannose, and/or galactose.

The compounds of the present disclosure and pharmaceutical compositions comprising at least one such compound may be useful in methods for treating and/or preventing thrombosis. As described herein methods are provided for inhibiting formation of a thrombus or inhibiting the rate at which a thrombus is formed. These methods may therefore be used for preventing thrombosis (i.e., reducing or decreasing the likelihood of occurrence of a thrombus in a statistically or clinically significant manner).

Thrombus formation may occur in infants, children, teenagers, and adults. An individual may have a hereditary predisposition to thrombosis. Thrombosis may be initiated, for example, due to a medical condition (such as cancer or pregnancy), a medical procedure (such as surgery), or an environmental condition (such as prolonged immobility). Other individuals at risk for thrombus formation include those who have previously presented with a thrombus.

The compounds of the present disclosure and pharmaceutical compositions comprising at least one such compound may be useful in methods for treating individuals undergoing thrombosis or who are at risk of a thrombotic event occurring. Such individuals may or may not have a risk of bleeding. In some embodiments, the individual has a risk of bleeding. In some embodiments, the thrombosis is a venous thromboembolism (VTE). VTE causes deep vein thrombosis and pulmonary embolism. Low molecular weight (LMW) heparin is the current mainstay therapy for the prevention and treatment of VTE. In many circumstances, however, the use of LMW heparin is contraindicated. LMW heparin is a known anti-coagulant and delays clotting over four times longer than control bleeding times. Patients undergoing surgery, patients with thrombocytopenia, patients with a history of stroke, and many cancer patients should avoid administration of heparin due to the risk of bleeding. By contrast, administration of the compounds of Formula (I) significantly reduces the time to clotting than occurs when LMW heparin is administered, and thus provide a significant improvement in reducing bleeding time compared with LMW heparin. Accordingly, the compounds and pharmaceutical compositions described herein may not only be useful for treating a patient for whom the risk of bleeding is not significant, but also may be useful in when the risk of bleeding is significant and the use of anti-thrombosis agents with anti-coagulant properties (such as LMW heparin) is contraindicated.

The compounds of the present disclosure and pharmaceutical compositions comprising at least one such compound may be administered in combination with at least one additional anti-thrombosis agent. The compounds of the present disclosure and pharmaceutical compositions comprising at least one such compound may function independently from the anti-thrombosis agent or may function in coordination with the at least one anti-thrombosis agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery and/or counteract side effects of administration of antibiotics or corticosteroids are examples of such palliative agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

The compounds of the present disclosure and pharmaceutical compositions comprising at least one such compound may be useful for treating and/or preventing at least one cardiovascular disease, disorder and/or condition. Non-limiting examples of cardiovascular disease include atherosclerosis, myocardial infarction, myocardial ischemia, coronary artery stenosis (occlusion of the coronary arteries), chronic cardiovascular and/or arterial inflammation, acute cardiovascular and/or arterial inflammation, hypercholesterolemia, restenosis (narrowing of the vessel lumen), arrhythmia, thrombosis, hyperlipidemia, hypertension, dyslipoproteinemia, angina (cardiac chest pain), and vascular complications due to a cardiovascular disease (e.g., myocardial infarction or myocardial ischemia).

In some embodiments, at least one compound of Formula (I) and/or a pharmaceutical composition comprising at least one compound of Formula (I) may be administered prior to or subsequent to an acute cardiovascular event in the subject. In some embodiments, at least one compound of Formula (I) and/or a pharmaceutical composition comprising at least one compound of Formula (I) may be administered prior to or subsequent to the development or diagnosis of a cardiovascular disease, disorder, and/or condition in the subject. In some embodiments, the acute cardiovascular event is a myocardial infarction.

In some embodiments, a method for treating and/or preventing atherosclerosis is disclosed, the method comprising administering to a subject in need thereof an effective amount of at least one compound of Formula (I) and/or a pharmaceutical composition comprising at least one compound of Formula (I). Atherosclerosis generally describes a disease of the arterial blood vessels. As used herein, “atherosclerosis” includes, but is not limited to, chronic and/or acute atherosclerotic inflammation prior to or subsequent to the formation of at least one atherosclerotic plaque in the subject. Atherosclerosis also includes, but is not limited to, chronic progressive atherosclerosis and/or atherosclerotic inflammation. Atherosclerosis also includes, but is not limited to, acute atherosclerosis and/or atherosclerotic inflammation subsequent to an acute vascular event in the subject (such as, for example, myocardial infarction).

In some embodiments, at least one compound of Formula (I) and/or a pharmaceutical composition comprising at least one compound of Formula (I) may be administered prior to or subsequent to the formation of at least one atherosclerotic plaque, lesion or atheroma in the subject.

In some embodiments, the formation, progression, destabilization, and/or rupture of at least one atherosclerotic plaque within the subject is reduced.

Atherosclerotic plaques may be characterized as stable or unstable (i.e., vulnerable to destabilization). Unstable atherosclerotic plaques may be susceptible to disruption or rupture, which exposes thrombogenic material (i.e., thrombi) (e.g., collagen) to the circulation. This can produce interruptions in blood flood (ischemia) in local or distal arteries, which can result in cardiovascular complications, such as, for example, myocardial infarction (MI).

Destabilization of atherosclerotic plaques may occur via many mechanisms. Non-limiting examples of such mechanisms include superficial erosion of the endothelial cells that form the monolayer covering the intima, disruption of the microvessels that form in the atherosclerotic plaque, rupture (i.e., fracture) of the atherosclerotic plaque's fibrous cap, thinning or weakening of the fibrous cap (thus making it susceptible to rupture), and the presence or increase in inflammatory factors that mediate destabilization. (Libby P., Nature, 420: 868-874 (2002)).

A non-limiting example of inflammatory factors that mediate destabilization is the presence of inflammatory cells. The progression of atherosclerosis may be associated with systemically increased inflammatory myeloid cells that are recruited to atherosclerotic plaques. (Murphy, A. J. et al., J. Clin. Invest., 121: 4138-4149 (2011); Averill, L. E. et al., Am. J. Pathol., 135: 369-377 (1989); Feldman, D. L. et al., Arterioscler. Thromb., 11: 985-994 (1991); Swirski, F. K. et al., J. Clin. Invest., 117: 195-205 (2007)). The presence of inflammatory myeloid cells may be detrimental to a stable plaque. (Llodra, J. et al., Proc. Natl. Acad. Sci. U.S.A., 101: 11779-11784 (2004)).

In some embodiments, the stability of at least one atherosclerotic plaque within the subject is increased. Non-limiting examples of stable features of atherosclerotic plaques (i.e., stable phenotype) include smaller plaque size, reduced (i.e., decreased, diminished, smaller) necrotic core size (measured by, for example, necrotic core area), and a thicker fibrous cap of the atherosclerotic plaque. (See, e.g., Moore K. J. et al., Cell, 145: 341-355 (2011)).

In some embodiments, the size of at least one atherosclerotic plaque within the subject is decreased. In some embodiments, the necrotic core size of at least one atherosclerotic plaque within the subject is decreased. In some embodiments, the fibrous cap thickness of at least one atherosclerotic plaque within the subject is increased.

In some embodiments, the administration of an effective amount of at least one compound of Formula (I) and/or a pharmaceutical composition comprising at least one compound of Formula (I) reduces the levels of extramedullary proliferation of hematopoietic stem and/or progenitor cells within the subject. In some embodiments, extramedullary proliferation of hematopoietic stem and/or progenitor cells is reduced in the spleen and/or the liver. Non-limiting examples of extramedullary proliferation of hematopoietic stem and/or progenitor cells include extramedullary hematopoiesis and extramedullary myelopoiesis.

In some embodiments, the administration of an effective amount of at least one compound of Formula (I) and/or a pharmaceutical composition comprising at least one compound of Formula (I) reduces the recruitment and/or infiltration of myeloid cells to at least one atherosclerotic plaque within the subject. Non-limiting examples of myeloid cells include monocytes, macrophages, neutrophils, basophils, eosinophils, erythrocytes, dendritic cells, and megakaryocytes and platelets.

In some embodiments, the at least one compound of Formula (I) and/or a pharmaceutical composition comprising at least one compound of Formula (I) is administered after angioplasty, stenting procedure, atherectomy, bypass surgery, or other vessel-corrective techniques.

In some embodiments, the at least one compound of Formula (I) and/or a pharmaceutical composition comprising at least one compound of Formula (I) is administered before angioplasty, stenting procedure, atherectomy, bypass surgery, or other vessel-corrective techniques.

In some embodiments, a method for treating and/or preventing myocardial infarction is disclosed, the method comprising administering to a subject in need thereof an effective amount of at least one compound of Formula (I) and/or a pharmaceutical composition comprising at least one compound of Formula (I). In some embodiments, the subject has previously suffered a myocardial infarction. In some embodiments, a compound of Formula (I) may be administered before the occurrence of a myocardial infarction in the subject. In some embodiments, a compound of Formula (I) may be administered after the occurrence of a first or subsequent myocardial infarction in the subject.

In some embodiments, at least one compound of Formula (I) and/or a pharmaceutical composition comprising at least one compound of Formula (I) is administered to the subject: within one (1) day of the subject suffering a myocardial infarction, within one (1) week of the subject suffering a myocardial infarction, within two (2) weeks of the subject suffering a myocardial infarction, within three (3) weeks of the subject suffering a myocardial infarction, within four (4) weeks of the subject suffering a myocardial infarction, within eight (8) weeks of the subject suffering a myocardial infarction, or within twelve (12) weeks of the subject suffering a myocardial infarction.

In some embodiments, a method for treating a disease, disorder, or condition associated with cardiac remodeling is disclosed, the method comprising administering to a subject in need thereof an effective amount of at least one compound of Formula (I) and/or a pharmaceutical composition comprising at least one compound of Formula (I).

In some embodiments, a method for treating sickle cell disease or complications associated therewith is disclosed, the method comprising administering to a subject in need thereof an effective amount of at least one compound of Formula (I) and/or a pharmaceutical composition comprising at least one compound of Formula (I).

In some embodiments, a method for treating and/or preventing vaso-occlusive crisis or complications associated therewith is disclosed, the method comprising administering to a subject in need thereof an effective amount of at least one compound of Formula (I) and/or a pharmaceutical composition comprising at least one compound of Formula (I).

In some embodiments, a method for treating and/or preventing pathological angiogenesis is disclosed, the method comprising administering to a subject in need thereof an effective amount of at least one compound of Formula (I) and/or a pharmaceutical composition comprising at least one compound of Formula (I).

In some embodiments, the pathological angiogenesis is in the eye. Non-limiting examples of ocular diseases, disorders, or conditions associated with pathological angiogenesis include age-related macular degeneration, ocular histoplasmosis syndrome, neovascular glaucoma, retrolental fibroplasia, pathologic myopia, angioid streaks, idiopathic disorders, choroiditis, choroidal rupture, overlying choroid nevi, graft rejection, herpes simplex keratitis, leishmaniasis, onchocerciasis, certain inflammatory diseases such as dry eye syndrome, and trauma to the eye (e.g., to the cornea).

In some embodiments, the present disclosure is directed to methods for the treatment and/or prevention of pathological angiogenesis in patients with cancer.

In some embodiments, a method for treating and/or preventing an epileptic syndrome is disclosed, the method comprising administering to a subject in need thereof at least one compound of Formula (I) and/or a pharmaceutical composition comprising at least one compound of Formula (I). Non-limiting examples of an epileptic syndrome include epilepsy, Rasmussen's syndrome, and West syndrome. Other syndromes which are multi-system disorders but with the primary disability resulting from neurological effects including epilepsy are considered epileptic syndromes for purposes of the present disclosure. A non-limiting example of such a syndrome is tuberous sclerosis syndrome.

The compounds of the present disclosure and pharmaceutical compositions comprising at least one such compound may be administered in combination with at least one additional antiepileptic agent (e.g., acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, rufinamide, sodium valproate, stiripentol, tiagabine, topiramate, vigabatrin, zonisamide). The compounds of the present disclosure and pharmaceutical compositions comprising at least one such compound may function independently from the antiepileptic agent or may function in coordination with the at least one antiepileptic agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery or enhancement of appetite, or counteract nausea or fatigue, are examples of such agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

In some embodiments, a method for treating and/or preventing a neurodegenerative disease is disclosed, the method comprising administering to a subject in need thereof an effective amount of at least one compound of Formula (I) and/or a pharmaceutical composition comprising at least one compound of Formula (I). Examples of neurodegenerative diseases include but are not limited to Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

In some embodiments, a method for treating and/or preventing α-synucleinopathies is disclosed, the method comprising administering to a subject in need thereof an effective amount of at least one compound of Formula (I) and/or a pharmaceutical composition comprising at least one compound of Formula (I). Non-limiting examples of α-synucleinopathies include Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy, and infantile neuroaxonal dystrophy.

The compounds of the present disclosure and pharmaceutical compositions comprising at least one such compound may be administered in combination with at least one additional agent for the treatment of neurodegeneration or symptoms thereof (e.g., donepezil, galantamine, memantine, rivastigmine, levodopa, carbidopa, dopamine agonists, COMT inhibitors, MAO inhibitors, anticholinergic agents, corticosteroids, beta interferons, ocrelizumab, glatiramer acetate, dimethyl fumarate, fingolimod, teriflunomide, natalizumab, alemtuzumab, mitoxantrone, riluzole, edaravone). The compounds of the present disclosure and pharmaceutical composition comprising at least one such compound may be administered before, after, or concurrently with administration of at least one additional agent for the treatment of neurodegeneration or symptoms thereof. Where administration is concurrent, the combination may be administered from a single container or two (or more) separate containers.

In some embodiments, a method for treating and/or preventing a fibrosing disease or condition is disclosed, the method comprising administering to a subject in need thereof an effective amount of at least one compound of Formula (I) and/or a pharmaceutical composition comprising at least one compound of Formula (I). Non-limiting examples of fibrosing diseases and conditions include rheumatoid arthritis, lupus, pathogenic fibrosis, fibrosing disease, heart disease, heart remodeling post MI, nonalcoholic fatty liver disease (NASH), idiopathic pulmonary fibrosis (IPF), fibrosis associated with thrombosis, fibrosis associated with macular degeneration, fibrotic lesions such as those formed after Schistosoma japonicum infection, radiation damage, autoimmune diseases, Lyme disease, chemotherapy induced fibrosis, HIV or infection-induced focal Sclerosis, failed back syndrome due to spinal Surgery scarring, abdominal adhesion post-Surgery scarring, fibrocystic formations, fibrosis after spinal injury, Surgery-induced fibrosis, mucosal fibrosis, peritoneal fibrosis caused by dialysis, Adalimumab-associated pulmonary fibrosis, and nephrogenic fibrosing dermopathy.

In some embodiments, the fibrosis is fibrosis of the liver resulting from conditions including but not limited to alcohol, drug, or chemically induced cirrhosis, ischemia-reperfusion injury after hepatic transplant, necrotizing hepatitis, hepatitis B, hepatitis C, primary biliary cirrhosis, primary sclerosing cholangitis, and nonalcoholic steatohepatitis.

In some embodiments, the fibrosis is fibrosis in the kidney resulting from conditions including but not limited to proliferative and Sclerosing glomerulonephritis, nephrogenic fibrosing dermopathy, diabetic nephropathy, renal tubulointerstitial fibrosis, and focal segmental glomerulosclerosis.

In some embodiments, the fibrosis is fibrosis of the lung resulting from conditions including but not limited to pulmonary interstitial fibrosis, sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema. There are several subnames or synonyms for pulmonary fibrosis including, but not limited to, cryptogenic fibrosing alveolitis, diffuse interstitial fibrosis, idiopathic interstitial pneumonitis, Hamman Rich syndrome, silicosis, asbestosis, berylliosis, coal worker's pneumoconiosis, coal miner's disease, miner's asthma, anthracosis, and anthracosilicosis.

In some embodiments, the fibrosis is fibrosis of the heart or pericardium resulting from conditions including but not limited to myocardial fibrosis, atherosclerosis, coronary artery restenosis, congestive cardiomyopathy, heart failure, and other post-ischemic conditions.

In some embodiments, the fibrosis is fibrosis of the eye resulting from conditions including but not limited to macular degeneration, exophthalmos of Grave's disease, proliferative vitreoretinopathy, anterior capsule cataract, corneal fibrosis, corneal scarring due to surgery, trabeculectomy-induced fibrosis, progressive sub-retinal fibrosis, multifocal granulomatous chorioretinitis, fibrosis due to wide angle glaucoma trabeculotomy, and other eye fibrosis.

In some embodiments, the fibrosis is fibrosis of the brain resulting from conditions including but not limited to glial scar tissue.

In some embodiments, the fibrosis is fibrosis of the skin resulting from conditions including but not limited to Depuytren's contracture, Scleroderma, keloid scarring, psoriasis, hyper-trophic scarring due to burns, atherosclerosis, restenosis, and pseudoscleroderma caused by spinal cord injury.

In some embodiments, the fibrosis is fibrosis of tissue including but not limited to the mouth or esophagus, pancreas, gastrointestinal tract, breast, bone, bone marrow, genitourinary system.

The terms “treat” and “treatment” include medical management of a disease, disorder, and/or condition of a subject as would be understood by a person of ordinary skill in the art (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or lessen an undesired physiological change or disorder, or to prevent or slow or lessen the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

In some embodiments of the methods described herein, the subject is a human. In some embodiments of the methods described herein, the subject is a non-human animal. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing diseases, disorders, and/or conditions treatable by inhibiting an activity of galectin-3 can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (I). In some embodiments, the pharmaceutical compositions further comprise at least one additional pharmaceutically acceptable ingredient.

In pharmaceutical compositions, any one or more of the compounds of the present disclosure may be administered in the form of a pharmaceutically acceptable derivative, such as a salt, and/or it or they may also be used alone and/or in appropriate association, as well as in combination, with other pharmaceutically active compounds.

An effective amount or therapeutically effective amount refers to an amount of at least one compound of the present disclosure or a pharmaceutical composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject. In general, the amount of at least one compound of Formula (I) as described herein, that is present in a dose, may range from about 0.1 mg to about 100 mg per kg weight of the subject. The minimum dose that is sufficient to provide effective therapy may be used in some embodiments. Subjects may generally be monitored for therapeutic effectiveness using assays suitable for the disease, disorder and/or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical compositions described herein may, for example, be sterile aqueous or sterile non-aqueous solutions, suspensions, or emulsions, and may additionally comprise at least one pharmaceutically acceptable excipient (i.e., a non-toxic material that does not interfere with the activity of the active ingredient). Such compositions may, for example, be in the form of a solid, liquid, or gas (aerosol). Alternatively, the compositions described herein may, for example, be formulated as a lyophilizate, or compounds described herein may be encapsulated within liposomes using technology known in the art. The pharmaceutical compositions may further comprise at least one additional pharmaceutically acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in compositions may be employed in the compositions described herein. Excipients for therapeutic use are well known, and are described, for example, in Remington: The Science and Practice of Pharmacy (Gennaro, 21^{st} Ed. Mack Pub. Co., Easton, PA (2005)). In general, the type of excipient may be selected based on the mode of administration, as well as the chemical composition of the active ingredient(s). Compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned ingredients, excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid composition may include, for example, at least one the following: a sterile diluent such as water for injection, saline solution, including for example physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition is an injectable composition, and in some embodiments, the injectable composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well-known technology and administered by, for example, oral, rectal, or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; the formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The pharmaceutical compositions may be formulated into pressurized acceptable propellants such as dichlorodifluoromethane, propane, nitrogen, and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound of Formula (I) and/or pharmaceutical composition comprising the same.

### EXAMPLES

### Example 1

### Synthesis of Compound 19

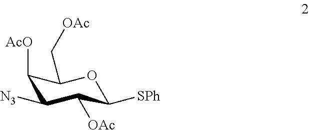

Compound 2: Compound 1 (1.5 g, 4.02 mmoles) was dissolved in DCM (30 mL). Thiophenol (0.9 g, 0.82 mL, 8.04 mmoles) was added, followed by dropwise addition of boron trifluoride diethyl etherate (1.79 g, 1.49 mL, 12.06 mmoles). The reaction mixture was stirred at room temperature for 2 days. The reaction quenched by addition of aqueous saturated NaHCO₃, transferred to a separatory funnel, and extracted 3 times with DCM. The combined organic phases were dried over sodium sulfate, filtered, and concentrated. The residue was purified by flash chromatography to give 2 as an off-white solid (1.1 g, 65% yield) LCMS (ESI): m/z calculated for C₁₈H₂₁N₃O₇S: 423.4, found 424.1 (M+1); 446.1 (M+Na).

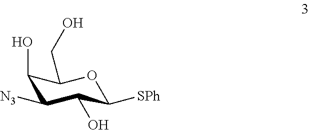

Compound 3: Compound 2 (1.1 g, 2.60 mmoles) was dissolved in methanol (25 mL) at room temperature. Sodium methoxide (0.1 mL, 25% sol. in MeOH) was added, and the reaction mixture was stirred at room temperature for 2 hours. The reaction mixture was neutralized by the addition of Amberlyst acidic resin, filtered and concentrated to give crude 3, which was used for the next step without further purification. LCMS (ESI): m/z calculated for C₁₂H₁₅N₃O₄S: 297.3, found 298.1 (M+1); 320.1 (M+Na).

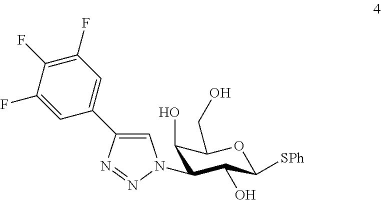

Compound 4: Crude compound 3 (2.60 mmoles), 3,4,5-trifluorophenyl-1-acetylene (2.5 equiv), THPTA (0.11 equiv), and copper (11) sulfate (0.1) were dissolved in methanol (15 mL) at room temperature. Sodium ascorbate (2.4 equiv) dissolved in water was added, and the reaction mixture was stirred overnight at room temperature. The resultant precipitate was collected by filtration, washed with hexanes and water, and dried to give compound 4 as a pale yellow solid (1.2 g, 100% yield for 2 steps). LCMS (ESI): m/z calculated for C₂₀H₁₈F₃N₃O₄S: 453.1, found 454.2 (M+1); 476.2 (M+Na).

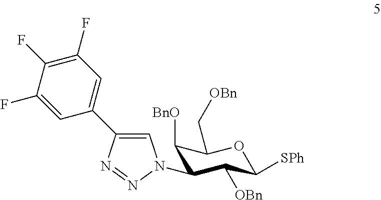

Compound 5: Compound 4 (1.2 g, 2.65 mmoles) was dissolved in DMF (15 mL) and cooled on an ice bath. Sodium hydride (60% oil dispersion, 477 mg, 11.93 mmoles) was added and the mixture stirred for 30 minutes. Benzyl bromide (1.42 mL, 11.93 mmoles) was added, and the reaction was warmed to room temperature and stirred overnight. The reaction mixture was quenched by the addition of aqueous saturated ammonium chloride solution, transferred to a separatory funnel, and extracted 3 times with ether. The combined organic phases were dried over magnesium sulfate, filtered, and concentrated. The residue was purified by flash chromatography to afford compound 5 (1.8 g, 94% yield). LCMS (ESI): m/z calculated for C₄₁H₃₆F₃N₃O₄S: 723.2, found 724.3 (M+1); 746.3 (M+Na).

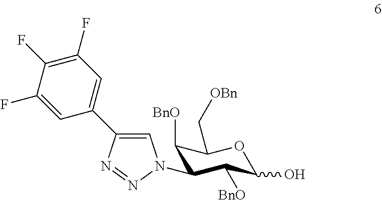

Compound 6: Compound 5 (1.8 g, 2.49 mmol) was dissolved in acetone (20 mL) and water (2 mL) and cooled on an ice bath. Trichloroisocyanuric acid (637 mg, 2.74 mmoles) was added, and the reaction mixture stirred on the ice bath for 3 hours. The acetone was removed in vacuo and the residue was diluted with DCM, transferred to a separatory funnel, and washed with saturated aqueous NaHCO₃. The organic phase was concentrated and the residue was purified by flash chromatography to afford compound 6 (1.5 g, 95%). LCMS (ESI): m/z calculated for C₃₅H₃₂F₃N₃O₅: 631.2, found 632.2 (M+1); 654.2 (M+Na).

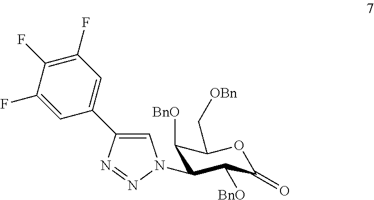

Compound 7: Compound 6 (1.0 g, 1.58 mmoles) was dissolved in DCM (20 mL) and cooled on an ice bath. Dess-Martin periodinane (1.0 g, 2.37 mmoles) was added, and the mixture was allowed to warm to room temperature and stirred overnight. The reaction mixture quenched by the addition of aqueous saturated NaHCO₃, transferred to a separatory funnel, and extracted 2 times with DCM. The combined organic phases were dried over sodium sulfate, filtered, and concentrated. The residue was purified by flash chromatography to afford compound 7 (520 mg, 52% yield). LCMS (ESI): m/z calculated for C₃₅H₃₀F₃N₃O₅: 629.2, found 652.2 (M+Na); 662.2 (M+MeOH+1); 684.2 (M+MeOH+Na).

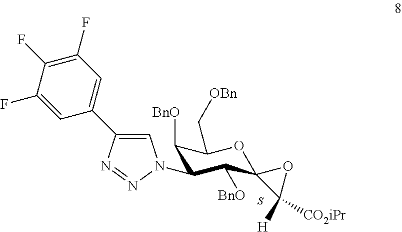

Compound 8: Isopropyl bromoacetate (6.44 g, 35.5 mmoles) was added dropwise to a solution of lithium bis(trimethylsilyl)amide (1.0 M in THF, 35.5 mL, 35.5 mmoles) cooled at −78° C. The reaction mixture was stirred for 25 minutes at −78 C. Compound 7 (5.6 g, 8.9 mmoles) dissolved in THF (80 mL) was then added dropwise. The reaction mixture was stirred at −78° C. for 2.5 hours then warmed to ice bath temperature. The reaction mixture was stirred for 30 minutes on the ice bath. The reaction mixture was then stirred at room temperature for 20 minutes. The reaction was quenched by the addition of aqueous saturated NH₄Cl. The reaction mixture was transferred to a separatory funnel and extracted 3 times with ethyl acetate. The combined organic phases were dried over sodium sulfate, filtered, and concentrated. The residue was separated by flash chromatography to afford compound 8 (6.3 g, 95% yield). MS (ESI): m/z calculated for C₄₀H₃₈F₃N₃O₇: 729.3, found 730.33 (M+H).

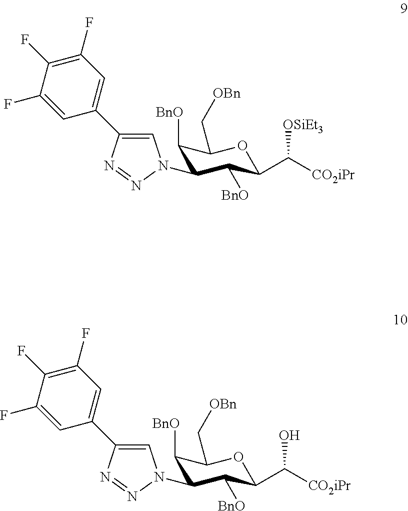

Compound 10: Compound 8 (6.3 g, 8.63 mmol) was dissolved in anhydrous CH₂Cl₂ (80 mL) and cooled on an ice bath while stirring under an atmosphere of argon. Tributyltin hydride (18.6 mL, 69.3 mmol) was added dropwise and the solution was allowed to stir for 10 minutes on the ice bath. Triethylsilyl triflate (3.3 mL, 14.7 mmol) was then added dropwise. The reaction was slowly warmed to ambient temperature and stirred for 24 hours. The reaction mixture was then diluted with CH₂Cl₂ (50 mL), transferred to a separatory funnel, and washed with saturated aqueous NaHCO₃ (50 mL). The aqueous phase was separated and extracted with CH₂Cl₂ (50 mL×2). The combined organic phases were dried over Na₂SO₄, filtered, and concentrated. The residue was purified by flash chromatography (hexanes to 40% EtOAc in hexanes, gradient) to afford compound 9 (1.6 g) and compound 10 (3.4 g). Compound 9: MS (ESI): m/z calculated for C₄₆H₅₄F₃N₃O₇Si: 845.4, found 846.4 (M+H). Compound 10: MS (ESI): m/z calculated for C₄₀H₄₇F₇N₃O₇: 731.3, found 732.3 (M+H).

Compound 9 (1.6 g, 1.86 mmol) was dissolved in THF (30 mL) under an Art atmosphere at room temperature. A solution of TBAF (1.9 mL, 1 M solution in THF, 1.9 mmol) was added dropwise. The reaction mixture was stirred at room temperature for 1.5 hours then concentrated under reduced pressure. The residue was separated by column chromatography to afford 0.72 g compound 9. This was combined with the above material to give 4.12 g compound 10. (65% yield). Compound 10 MS (ESI): m/z calculated for C₄₀H₄₀F₃N₃O₇: 731.3, found 732.3 (M+H).

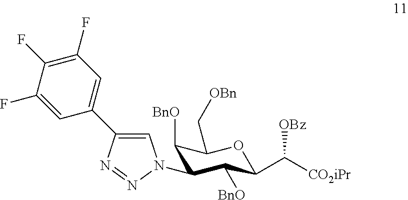

Compound 11: Compound 10 (4.1 g, 5.63 mmol) was dissolved in anhydrous CH₂Cl₂ (60 mL) under an atmosphere of argon. Pyridine (4.55 mL, 56 mmol) was added followed by benzoyl chloride (2.61 mL, 22 mmol). The reaction mixture was stirred for 20 hours at room temperature. The reaction mixture was transferred to a separatory funnel and washed with 1 M HCl (100 mL). The aqueous phase was separated and extracted with CH₂Cl₂ (60 mL×3). The combined organic phases were dried over Na₂SO₄, filtered, and concentrated. The residue was separated by column chromatography to afford compound 11 (4.19 g). (89% Yield) MS (ESI): m/z calculated for C₄₇H₄₄F₃N₃O₅: 835.3, found 836.1 (M+H).

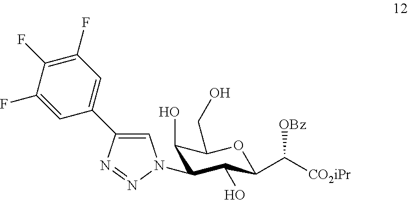

Compound 12: To a solution of compound 11 (4.2 g, 5.0 mmol) in anhydrous MeOH (60 mL) was added Pd/C (0.6 g, 20% by wt). The mixture was degassed, then stirred under a hydrogen atmosphere overnight. The reaction mixture was filtered through a 25 mm 0.45 μm PTFE membrane and concentrated to afford compound 12 (2.7 g, 95%). MS (ESI): m/z calculated for C₂₆H₂₆F₃N₃O₈: 565.2, found 566.2 (M+H).

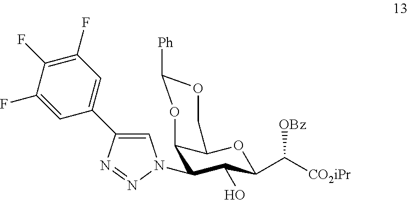

Compound 13: Compound 12 (2.7 g, 5 mmol) was dissolved in acetonitrile (45 mL). Benzaldehyde dimethyl acetal (1.11 mL, 7.45 mmol) was added, followed by camphorsulfonic acid (115 mg, 0.49 mmol). The reaction mixture was stirred for 3 hours at 43° C. The reaction mixture colled on an ice bath and neutralized with Et₃N (1 mL). The reaction mixture was stirred for 10 minutes on the ice bath then concentrated. The residue was separated via flash chromatography to afford compound 13 (2.87 g, 88%). MS (ESI): m/z calculated for C₃₃H₃₀F₃N₃O₈: 653.2, found 654.1 (M+H).

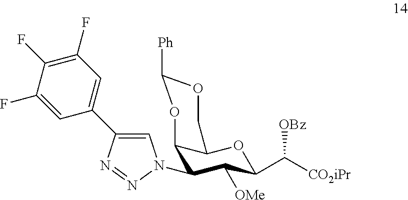

Compound 14: Compound 13 (2.84 g, 4.34 mmol) dissolved in anhydrous DMF (40 mL) and cooled on an ice bath. Iodomethane (2.7 mL, 43.4 mmol) was added, followed by sodium hydride (191 mg, 4.77 mmol, 60% dispersion in mineral oil). The reaction mixture was stirred 4 hours on the ice bath, then quenched by the addition of saturated ammonium sulfate solution (5 mL). The reaction mixture was diluted with DCM (100 mL), transferred to a separatory funnel, and washed with H₂O (50 mL). The aqueous phase was separated and extracted with DCM (100 mL×3). The combined organic phases were dried over Na₂SO₄, filtered, and concentrated. The residue was separated by column chromatography to afford compound 14 (2.59 g, 89%). MS (ESI): m/z calculated for C₃₄H₃₂F₃N₃O₈: 667.2, found 668.1 (M+H).

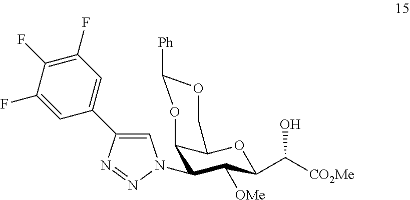

Compound 15: Compound 14 (2.59 g, 3.87 mmol) dissolved in a solution of sodium methoxide in MeOH (130 mL, 75 mM) at room temperature. The reaction mixture was stirred for 84 hours. The pH of the solution was adjusted to 7 by the addition of Amberlite IRC 120H (hydrogen form) resin. The reaction mixture was filtered, and the filtrate concentrated to afford compound 15 (1.36 g, 66%). MS (ESI): m/z calculated for C₂₅H₂₄F₃N₃O₇: 535.2, found 536.2 (M+H).

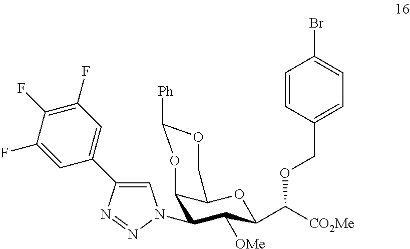

Compound 16: Compound 15 (1.36 g, 2.53 mmol) and 4-bromobenzyl bromide (1.9 g, 7.61 mmol) were dissolved in anhydrous DMF (40 mL) and cooled on an ice bath. Sodium hydride (111 mg, 2.79 mmol, 60% dispersion in mineral oil) was added, and the reaction mixture was stirred 2 hours on the ice bath. The reaction mixture was quenched by the addition of saturated ammonium sulfate solution (2 mL). The reaction mixture was diluted with DCM (100 mL), transferred to a separatory funnel, and washed with H₂O (100 mL). The aqueous phase was separated and extracted with DCM (100 mL×3). The combined organic phases were dried over Na₂SO₄, filtered, and concentrated. The residue was separated by column chromatography to afford compound 16 (1.54 g, 86%). MS (ESI): m/z calculated for C₃₂H₂₉BrF₃N₃O₇: 703.1, found 704.1, 706.1 (M+H).

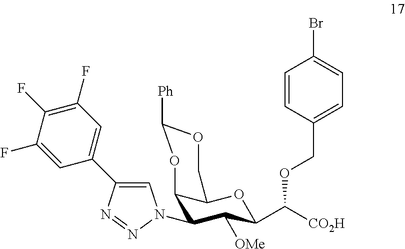

Compound 17: Compound 16 (1.54 g, 2.18 mmol) was dissolved in THF (40 mL) and cooled on an ice bath. An aqueous solution of LiOH (370 mg LiOH, 15.4 mmol, in 20 mL of water) was added, and the reaction mixture was stirred 8.5 hours on the ice bath. The reaction mixture was diluted with EtOAc (100 mL), transferred to a separatory funnel, and washed with 1M HCl (100 mL). The aqueous phase was separated and extracted with EtOAc (100 mL×3). The combined organic phases were dried over Na₂SO₄, filtered, and concentrated to afford compound 17 (1.5 g, 100%/). MS (ESI). m/z calculated for C₃₁H₂₇BrF₃N₃O₇: 689.1, found 690.1, 692.1 (M+H).

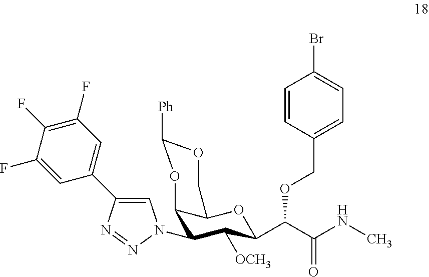

Compound 18: To a solution of compound 17 (1.5 g, 2.17 mmol) dissolved in DCM (60 mL) under an argon atmosphere at room temperature was added HATU (1.87 g, 4.34 mmol) followed by DIPEA (3 mL, 17.2 mmol) followed by methylamine (2.46 mL, 2 M MeNH₂ in THF, 4.92 mmol). The reaction mixture was stirred 4.5 hours. The reaction mixture was diluted with DCM (100 mL), transferred to a separatory funnel, and washed with 0.25 M HCl (100 mL). The aqueous phase was separated and extracted with DCM (50 mL×3). The combined organic phases were washed with saturated NaHCO₃ (100 mL), dried over Na₂SO₄, filtered, and concentrated. The residue was separated by column chromatography to afford compound 18 (1.5 g, 98%). MS (ESI): m/z calculated for C₃₂H₃₀BrF₃N₄O₇: 702.1, found 703.1, 705.1 (M+H).

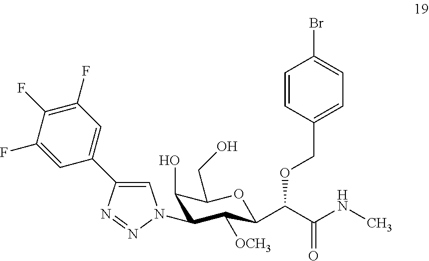

Compound 19: To a solution of compound 18 (1.2 g, 1.7 mmol) dissolved in 1,4-dioxane (40 mL) was added HCl (1.2 M, 40 mL). The reaction mixture was stirred 9 hours at 50° C. The reaction mixture was cooled to room temperature and diluted with DCM (100 mL). The reaction mixture was transferred to a separatory funnel, and extracted with DCM (50 mL×3). The combined organic phases were washed with saturated NaHCO₃ (50 mL), dried over Na₂SO₄, filtered, and concentrated. The residue was separated by column chromatography to afford compound 19 (738 mg, 70%).

¹H NMR (400 MHz, CH₃OH-d₄) δ ppm 8.67 (s, 1H), 7.72-7.63 (m, 2H), 7.54 (d, J=8 Hz, 2H), 7.41 (d, J=8 Hz, 2H), 4.93 (dd, J=12, 4 Hz, 1H), 4.70 (d, J=12 Hz, 1H), 4.47 (d, J=12 Hz, 1H), 4.27-4.18 (m, 2H), 4.06 (d, J=4 Hz, 1H), 3.74-3.61 (m, 4H), 2.93 (s, 3H), 2.81 (s, 3H). LCMS (ESI): m/z calculated for C₂₅H₂₆BrF₃N₄O₇: 614.1, found 615.0, 617.0 (M+H).

The following compounds were prepared as outlined in FIG. 1.

### Example 2

### Synthesis of Compound 20

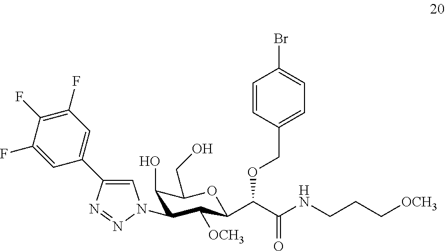

Compound 20: Compound 20 was prepared according to FIG. 1 using 3-methoxypropylamine in step p.

¹H NMR (400 MHz, Methanol-d4) δ 8.59 (d, J=1.4 Hz, 1H), 7.58 (t, J=7.6 Hz, 2H), 7.49-7.41 (m, 2H), 7.33 (d, J=7.8 Hz, 2H), 4.85 (dd, J=10.9, 2.5 Hz, 1H), 4.61 (d, J=11.6 Hz, 1H), 4.40 (d, J=11.6 Hz, 1H), 4.15 (t, J=10.0 Hz, 1H), 4.10 (s, 1H), 3.98 (d, J=2.5 Hz, 1H), 3.64 (d, J=9.4 Hz, 1H), 3.57 (q, JJ=5.8 Hz, 3H), 3.36 (t, J=6.1 Hz, 2H), 3.29-3.23 (m, 4H), 2.86 (d, J=1.4 Hz, 3H), 1.69 (q, J=6.5 Hz, 2H). LCMS (ESI): m/z calculated for C₂₉H₃₂BrF₃N₄O₇: 672.1, found 674.1 (M+H).

### Example 3

### Synthesis of Compound 21

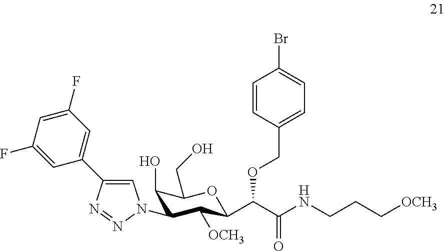

Compound 21: Compound 21 was prepared according to FIG. 1 using 3,5-difluorophenyl-1-acetylene in step c and 3-methoxypropylamine in step p.

¹H NMR (400 MHz, MeOD) δ 8.61 (d, J=1.6 Hz, 1H), 7.49-7.38 (m, 4H), 7.33 (d, J=8.0 Hz, 2H), 6.88-6.79 (m, 1H), 4.88-4.81 (m, 1H), 4.61 (d, J=11.6 Hz, 1H), 4.41 (d, J=11.6 Hz, 1H), 4.17 (t, J=10.0 Hz, 1H), 4.10 (s, 1H), 3.99 (s, 1H), 3.64 (d, J=9.4 Hz, 1H), 3.57 (q, J=5.7 Hz, 3H), 3.36 (t, J=6.1 Hz, 2H), 3.26 (t, J=6.7 Hz, 2H), 3.21 (d, J=1.6 Hz, 4H), 2.87 (d, J=1.6 Hz, 3H), 1.69 (p, J=6.6 Hz, 2H). LCMS (ESI): m/z calculated for C₂₈H₃₃BrF₂N₄O₇: 654.1, found 655.1, 657.1 (M+H).

### Example 4

### Synthesis of Compound 22

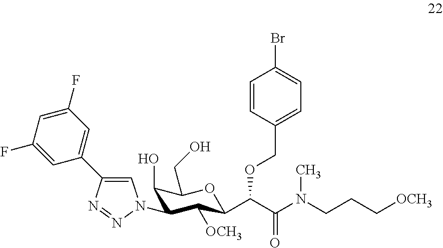

Compound 22: Compound 22 was prepared according to FIG. 1 using 3,5-difluorophenyl-1-acetylene in step c and N-methyl-3-methoxypropylamine in step p.

¹H NMR (400 MHz, MeOD) δ 8.65 (s, 1H), 7.48-7.38 (m, 4H), 7.31 (t, J=8.1 Hz, 2H), 6.84 (dt, J=10.3, 5.1 Hz, 1H), 4.84 (d, J=11.6 Hz, 1H), 4.71 (s, OH), 4.56 (d, J=11.9 Hz, 2H), 4.32 (t, J=12.3 Hz, 1H), 4.22-4.13 (m, 1H), 3.95 (d, J=5.5 Hz, 1H), 3.67 (d, J=9.4 Hz, 1H), 3.59 (d, J=8.1 Hz, 2H), 3.48 (q, J=6.8 Hz, OH), 3.32 (dt, J=12.4, 6.3 Hz, 3H), 3.23 (d, J=9.3 Hz, 4H), 3.07 (s, 2H), 2.91 (d, J=11.7 Hz, 4H), 1.77 (q, =8.3, 7.2 Hz, 2H). LCMS (ESI): m/z calculated for C₂₉H₃₅BrF₂N₄O₇: 668.2, found 669.1, 671.1 (M+H).

### Example 5

### Synthesis of Compound 23

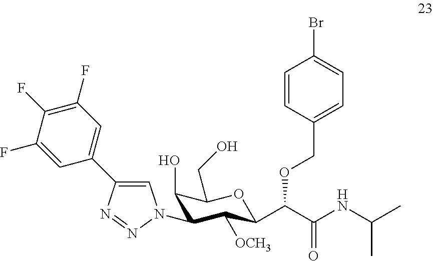

Compound 23: Compound 23 was prepared according to FIG. 1 using isopropylamine in step p.

¹H NMR (400 MHz, MeOD) δ 8.59 (s, 1H), 7.58 (dd, J=8.8, 6.6 Hz, 2H), 7.54-7.40 (m, 2H), 7.35-7.20 (m, 2H), 4.83 (dd, J=10.6, 2.9 Hz, 1H), 4.57 (d, J=11.6 Hz, 1H), 4.41 (d, J=11.6 Hz, 1H), 4.16 (dd, J=10.6, 9.4 Hz, 1H), 4.07 (d, J=2.1 Hz, 1H), 4.03-3.89 (m, 2H), 3.74-3.44 (m, 4H), 2.87 (s, 3H), 1.07 (dd, J=6.6, 1.4 Hz, 6H). LCMS (ESI): m/z calculated for C₂₇H₃₀BrF₃N₄O₆: 642.1, 644.1, found 642.9, 644.9 (M+H).

### Example 6

### Synthesis of Compound 27

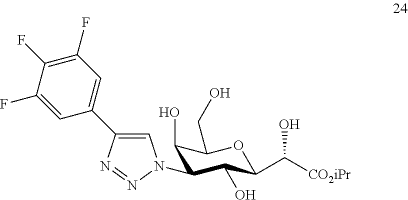

Compound 24: To a solution of compound 10 (1.1 g, 1.5 mmol) in anhydrous iPrOH (25 mL) and EtOAc (25 mL) was added Pd/C (0.2 g, 10% by wt). The mixture was degassed. Triethyl silane (0.9 mL, 5.4 mmol) was added, and the reaction mixture was stirred at room temperature. Aliquots of triethylsilane (0.9 mL, 5.4 mmol) were added until the reaction was complete. The reaction mixture was filtered through Celite and concentrated. The residue was separated by column chromatography to afford compound 24 (160 mg, 23%). MS (ESI): m/z calculated for C₁₉H₂₂F₃N₃O₇: 461.1, found 484.1 (M+Na).

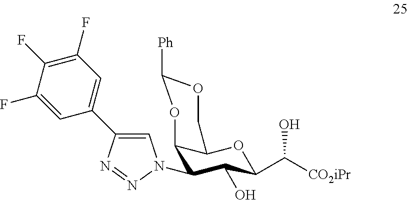

Compound 25: To a suspension of compound 24 (233 mg, 0.50 mmol) in anhydrous acetonitrile (5 mL) was added camphorsulfonic acid (23 mg, 0.1 mmol) and benzaldehyde dimethyl acetal (83 μL, 0.55 mmol). The reaction mixture was stirred under argon at 60° C. for 2 hours (the reaction mixture became homogenous after about 30 minutes). The reaction mixture was quenched by the addition of triethylamine (0.2 mL) and concentrated. The residue was separated by column chromatography to afford compound 25 as an off-white solid (167 mg, 60%). MS (ESI): m/z calculated for C₂₆H₂₆F₃N₃O₇: 549.2, found 572.1 (M+Na).

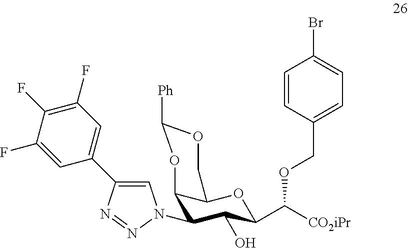

Compound 26: Compound 25 (167 mg, 0.30 mmol) was dissolved in anhydrous DMF (1 mL) under an argon atmosphere and cooled to −10° C. Sodium iodide (50 mg, 0.3 mmol) and 4-bromobenzyl bromide (76 mg, 0.33 mmol) were added. Sodium hydride (13 mg of a 60% oil dispersion, 0.33 mmol) was added and the reaction mixture was stirred for 2 hours at −10° C. The reaction mixture was quenched by the addition of saturated ammonium chloride solution (5 mL). The reaction mixture was transferred to a separatory funnel and extracted with ethyl acetate (3×10 mL). The combined organic extracts were washed with water and concentrated. The residue was separated by column chromatography to afford compound 26 (149 mg, 60%). MS (ESI): m/z calculated for C₃₃H₃₁BrF₃N₃O₇: 717.1, found 718.2, 720.1 (M+H).

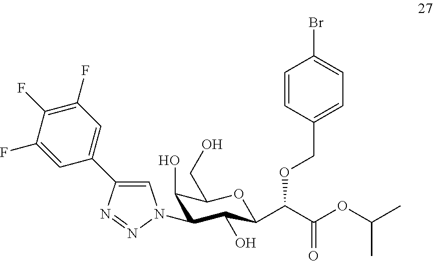

Compound 27: To a solution of compound 26 (149 mg, 0.21 mmol) dissolved in 1,4-dioxane (3 mL) was added 50% HCl solution (1.5 mL). The reaction mixture was stirred for 2 hours at 50° C. The reaction mixture was cooled to room temperature and concentrated. The residue was separated by column chromatography to afford compound 27 (24 mg, 18%).

¹H NMR (400 MHz, d6-DMSO) δ 8.77 (s, 1H), 7.88 (dd, 0.1=9.2, 6.7 Hz, 2H), 7.61-7.53 (m, 2H), 7.44-7.36 (m, 2H), 5.43 (d, J=7.6 Hz, 1H), 5.24 (d, J=6.0 Hz, 1H), 5.01 (p, J=6.2 Hz, 1H), 4.83 (dd, J=10.7, 2.9 Hz, 1H), 4.62 (d, J=11.4 Hz, 1H), 4.56 (t, J=5.5 Hz, 1H), 4.49 (d, J=11.4 Hz, 1H), 4.36 (d, J=2.5 Hz, 1H), 4.35-4.26 (m, 1H), 3.95-3.88 (m, 1H), 3.77 (dd, J=9.4, 2.6 Hz, 1H), 3.55 (t, J=6.4 Hz, 1H), 3.51-3.41 (m, 1H), 3.37 (dt, J=10.8, 5.5 Hz, 1H), 1.23 (dd, J=6.3, 4.8 Hz, 6H). MS (ESI): m/z calculated for C₂₆H₂₇BrF₃N₃O₇: 629.1, found 629.9, 631.8 (M+H).

### Example 7

### Synthesis of Compound 35

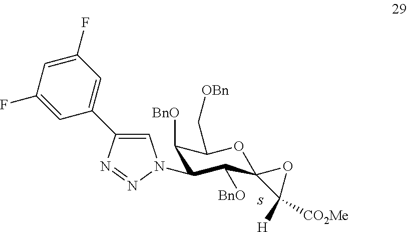

Compound 29: Methyl bromoacetate (2.2 g, 23 mmoles) dissolved in 10 mL of THF was added dropwise to a solution of lithium bis(trimethylsilyl)amide (1.0 M in THF, 23 mL, 23 mmoles) cooled at −78° C. The reaction mixture was stirred for 30 minutes at −78° C. Compound 28 (2.7 g, 5.7 mmoles, prepared according to FIG. 1 using 3,5-difluorophenyl acetylene in step c) dissolved in THF (2.0 mL) was then added. The reaction mixture was stirred at −78° C. for 30 minutes. The reaction was quenched by the addition of aqueous saturated NHaCl and warmed to room temperature. The reaction mixture was transferred to a separatory funnel and extracted 3 times with ethyl acetate. The combined organic phases were dried over sodium sulfate, filtered, and concentrated. The residue was separated by flash chromatography to afford compound 29 (2.4 g, 58% yield). LCMS (ESI): m/z calculated for C₃₈H₃₅F₂N₃O₇: 715.2, found 738.1 (M+Na).

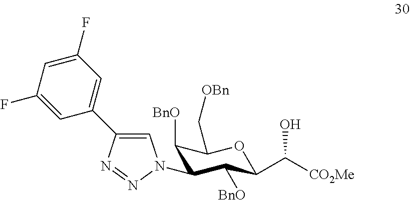

Compound 30: Compound 29 (1.9 g, 2.7 mmol) was azeotroped with toluene two times. It was then dissolved in anhydrous CH₂Cl₂ (30 mL) and cooled to −20° C. under an atmosphere of argon. Triethylsilane (1.8 mL, 11 mmol) was added dropwise. Triethylsilyl triflate (21.6 mL, 8.2 mmol) was then added dropwise. The reaction was stirred 2 hours at −20° C. The reaction mixture was quenched by the addition of saturated NaHCO₃ solution. The reaction mixture was transferred to a separatory funnel and the phases separated. The aqueous phase was separated and extracted with CH₂Cl₂ (50 mL×2). The combined organic phases were washed with saturated aqueous NaHCO₃ (50 mL), dried over Na₂SO₄, filtered, and concentrated. The residue was purified by column chromatography to afford compound 30 (760 mg, 41%). LCMS (ESI): m/z calculated for C₃₈H₃₇F₂N₃O₇: 685.3, found 708.3 (M+Na).

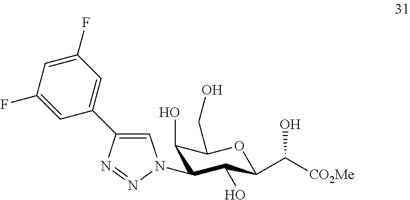

Compound 31: To a mixture of compound 30 (760 mg, 1.1 mmol) dissolved in a mixture of MeOH (5 mL) and THF (5 mL) was added Pd/C (0.2 g, 20% by wt). The mixture was hydrogenated on a Paar apparatus at 50 psi overnight. The reaction mixture was filtered through a bed of Celite and concentrated. The residue was purified by column chromatography to afford compound 31 (169 mg, 37%). LCMS (ESI): m/z calculated for C₁₇H₁₉F₂N₃O₇: 415.1, found 438.1 (M+Na).

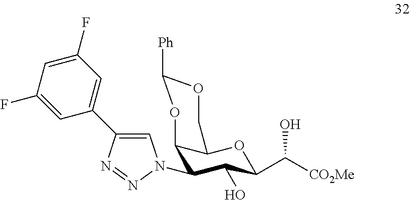

Compound 32: Compound 31 (146 mg, 0.35 mmol) and CSA (8 mg, 0.035 mmol) were suspended in anhydrous acetonitrile (2 mL) under an argon atmosphere. Benzaldehyde dimethyl acetal (58 μL, 0.39 mmol) was added. The reaction mixture was stirred at 60° C. for 3 hours. It became homogenous after about 2 minutes. The reaction mixture was then neutralized with a few drops of Et₃N, and concentrated. The residue was separated via column chromatography to afford compound 32 (133 mg, 75%). LCMS (ESI): m/z calculated for C₂₄H₂₃F₂N₃O₇: 503.2, found 526.1 (M+Na).

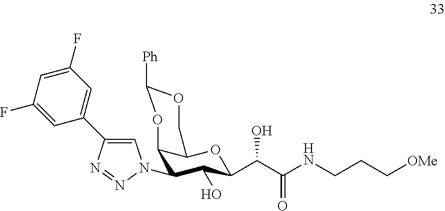

Compound 33: Compound 32 (172 mg, 0.14 mmol) was dissolved in dry THF (1 mL) under an argon atmosphere. 3-Methoxypropylamine (73 μL, 0.72 mmol) was added, and the reaction mixture was stirred at 50° C. overnight. The reaction mixture was adsorbed onto silica gel and separated via column chromatography to afford compound 33 (57 mg, 73%). LCMS (ESI): m/z calculated for C₂₇H₃₀F₂N₃O₇: 560.2, found 583.2 (M+Na).

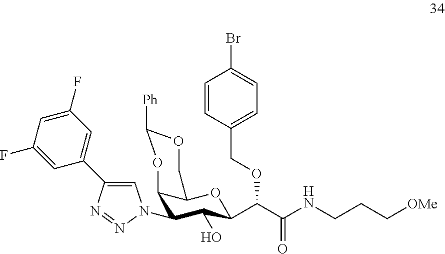

Compound 34: Compound 33 (22 mg, 0.039 mmol) was dissolved in anhydrous DMF (1 mL) under an argon atmosphere. 4-Bromobenzyl bromide (11 mg, 0.043 mmol) was added and the reaction mixture was cooled on an ice bath. Sodium hydride (2 mg of a 60% oil dispersion, 0.058 mmol) was added, and the reaction mixture was stirred for 1 hour on the ice bath. The reaction mixture was diluted with EtOAc, transferred to a separatory funnel, and washed with saturated NHaCl solution followed by water. The organic phase was concentrated. The residue was separated via column chromatography to afford compound 34 (10 mg, 36%). LCMS (ESI): m/z calculated for C₃₄H₃₅BrF₂N₃O₇: 728.2, found 753.1 (M+Na).

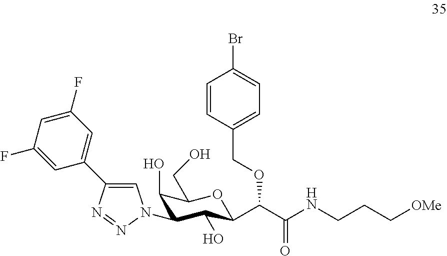

Compound 35: Compound 34 (10 mg, 0.014 mmol) was dissolved in MeOH (1 mL) and water (0.2 mL). Camphorsulfonic acid (2 mg, 0.008 mmol) was added, and the reaction mixture was stirred at 60° C. overnight. The reaction mixture was adsorbed onto silica gel and separated by column chromatography to afford compound 35 (7 mg, 74%).

¹H NMR (400 MHz, MeOD) δ 8.56 (d, J=1.5 Hz, 1H), 7.60-7.49 (m, 4H), 7.44 (d, J=8.0 Hz, 2H), 6.96 (dt, J=10.8, 5.4 Hz, 1H), 4.86 (d, J=2.9 Hz, 1H), 4.67 (s, 2H), 4.55 (t, J=10.1 Hz, 1H), 4.34 (d, J=2.1 Hz, 1H), 4.16 (d, J=2.3 Hz, 1H), 3.83-3.75 (m, 1H), 3.70 (d, J=1.6 Hz, 3H), 3.49-3.42 (m, 2H), 3.39-3.29 (m, 6H), 1.79 (p, J=6.3 Hz, 2H). LCMS (ESI): m/z calculated for C₂₇H₃₁BrF₂N₃O₇: 640.1, found 641.1, 643.1 (M+H).

### Example 8

### Synthesis of Compound 36

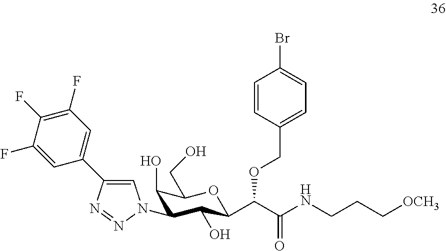

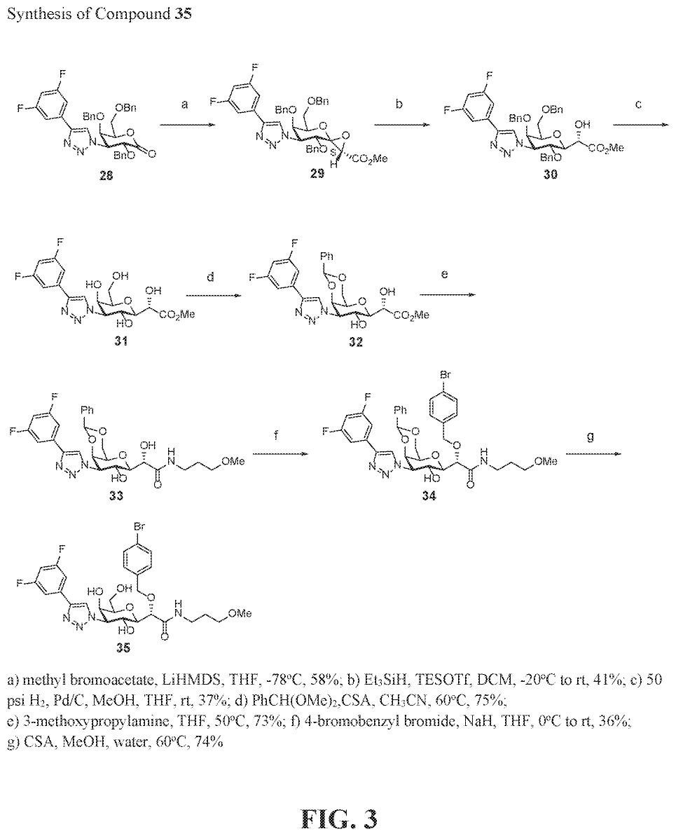

Compound 36: Compound 36 was prepared according to FIG. 3 using compound 7 as the starting material in step a.

¹H NMR (400 MHz, Methanol-d4) δ 8.42 (d, J=1.1 Hz, 1H), 7.63-7.53 (m, 2H), 7.49-7.42 (m, 2H), 7.32 (d, J=7.9 Hz, 2H), 4.76-4.72 (m, 1H), 4.54 (s, 2H), 4.42 (t, J=10.1 Hz, 1H), 4.21 (d, J=1.8 Hz, 1H), 4.03 (d, J=2.8 Hz, 1H), 3.66 (dd, J=9.4, 1.9 Hz, 1H), 3.58 (s, 3H), 3.33 (t, J=6.1 Hz, 2H), 3.24 (d, J=6.5 Hz, 2H), 3.19 (d, J=1.1 Hz, 3H), 1.67 (p, J=6.4 Hz, 2H). LCMS (ESI): m/z calculated for C₂₇H₃₀BrF₃N₄O₂: 658.1, found 666.1 (M+H).

### Example 9A

### Prophetic Synthesis of Compound 43

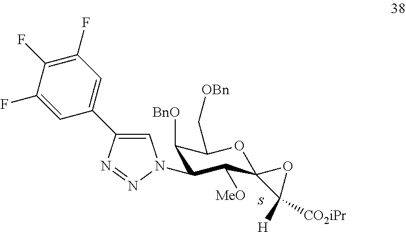

Compound 38: Isopropyl bromoacetate dissolved in THF is added dropwise to a solution of lithium bis(trimethylsilyl)amide (1.0 M in THF, 1 equivalent) cooled to −78° C. The reaction mixture is stirred for 30 minutes at −78° C. Compound 37 (0.25 equivalents, prepared according to WO2020/139960) dissolved in THF is then added. The reaction mixture is stirred at −78° C. until completion. The reaction is quenched by the addition of aqueous saturated NH₄Cl and warmed to rt. The reaction mixture is transferred to a separatory funnel and extracted 3 times with ethyl acetate. The combined organic phases are dried over sodium sulfate, filtered, and concentrated. The residue is separated by flash chromatography to afford compound 38.

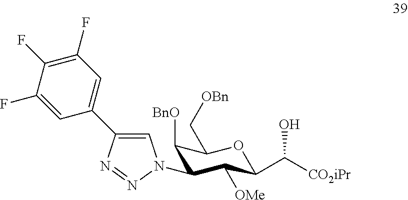

Compound 39: Compound 38 is azeotroped with toluene two times. It is then dissolved in anhydrous CH₂Cl₂ and cooled to −20° C. under an atmosphere of argon. Triethylsilane (4 equivalents) is added dropwise. Triethylsilyl triflate (3 equivalents) is then added dropwise. The reaction is stirred at −20° C. until completion. The reaction mixture is quenched by the addition of saturated NaHCO₃ solution. The reaction mixture is transferred to a separatory funnel and the phases separated. The aqueous phase is separated and extracted with CH₂Cl₂. The combined organic phases are washed with saturated aqueous NaHCO₃, dried over Na₂SO₄, filtered, and concentrated. The residue is purified by column chromatography to afford compound 39.

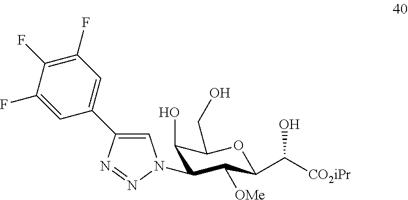

Compound 40: To a mixture of compound 39 dissolved in a mixture of MeOH and THF is added Pd/C. The mixture is hydrogenated on a Paar apparatus at 50 psi until completion. The reaction mixture is filtered through a bed of Celite and concentrated. The residue is purified by column chromatography to afford compound 40.

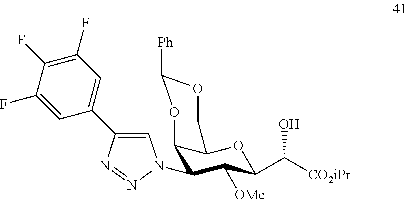

Compound 41: Compound 40 and CSA (0.1 equivalents) are suspended in anhydrous acetonitrile under an argon atmosphere. Benzaldehyde dimethyl acetal (1.1 equivalents) is added. The reaction mixture is stirred at 60° C. until completion. The reaction mixture is neutralized with Et₃N, and concentrated. The residue is separated via column chromatography to afford compound 41.

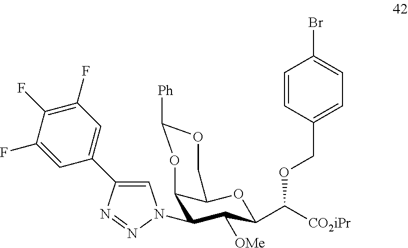

Compound 42: Compound 41 (167 mg, 0.30 mmol) is dissolved in anhydrous DMF under an argon atmosphere and cooled to −10° C. Sodium iodide (1 equivalent) and 4-bromobenzyl bromide (1.1 equivalents) are added. Sodium hydride (1.1 equivalent of a 60% oil dispersion) is added, and the reaction mixture is stirred at −10° C. until completion. The reaction mixture is quenched by the addition of saturated ammonium chloride solution. The reaction mixture is transferred to a separatory funnel and extracted with ethyl acetate. The combined organic extracts are washed with water and concentrated. The residue is separated by column chromatography to afford compound 42.

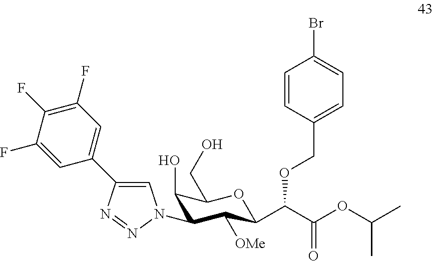

Compound 43: To a solution of compound 42 dissolved in 1,4-dioxane was added 50% HCl solution. The reaction mixture was stirred at 50° C. until completion. The reaction mixture is cooled to room temperature and concentrated. The residue is separated by column chromatography to afford compound 43.

### Example 9B

### Synthesis of Compound 43

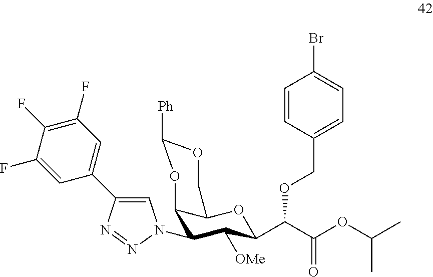

Compound 42: Compound 17 (51 mg, 74 μmole), isopropanol (5.7 μL, 740 μmole) and DMAP (1.8 mg, 1.5 μmole) were dissolved in DCM (2 mL) and the solution was stirred for 5 min. at ambient temperature. DCC (20 mg, 96 μmole) was added in one portion and the resulting solution was stirred for 7 hrs. The solution was filtered through a Celite pad and the filtrate was concentrated. The reaction mixture was separated by chromatography to afford compound 42 (18 mg, 34%). MS (ESI): m/z calculated for C₃₄H₃₃BrF₃N₃O₇: 731.1, 733.1; found 754.1, 756.1 (M+Na).

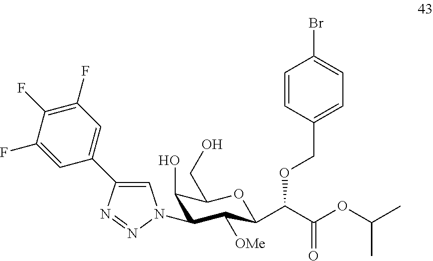

Compound 43: To a solution of compound 42 (25 mg, 34 μmole) in 1,4-dioxane (1 mL) was added aqueous HCl solution (25%, 0.2 mL). The solution was stirred for 2 hrs at 55° C. After completion of the reaction, the solution was cooled to 0° C. and then neutralized by addition of saturated aqueous NaHCO₃ to pH 7. The solution was concentrated and the residue was separated by chromatography to afford compound 43 (20 mg, 91%).

¹H NMR (400 MHZ, METHANOL-D₄) Δ 8.71 (S, 1H), 7.68 (T, J=8.0 HZ, 2H), 7.54 (D, J=8.2 HZ, 2H), 7.42 (D, J=8.1 HZ, 2H), 5.17 (P, J=6.2 HZ, 1H), 4.93 (DD, J=10.6, 2.9 HZ, 1H), 4.83 (D, J=11.1 HZ, 1H), 4.48 (D, J=11.1 HZ, 1H), 4.43 (D, J=4.0 HZ, 1H), 4.24 (T, J=10.1 HZ, 1H), 4.06 (D, J=3.0 HZ, 1H), 3.89 (DD, J=9.4, 2.5 HZ, 1H), 3.71-3.62, M, 3H), 3.04 (S, 3H), 1.40-1.23 (M, 6H); MS (ESI): M/Z CALCULATED FOR C₂₇H₂₉BRF₃N₃O₇: 643.1, 645.1; FOUND 644.0, 646.0 (M+H).

### Example 10A

### Prophetic Synthesis of Compound 44

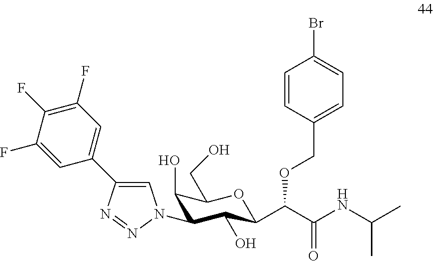

Compound 44: Compound 44 can be prepared according to FIG. 3 by using compound 7 instead of compound 28 and using isopropylamine in step e.

### Example 10b

### Synthesis of Compound 44

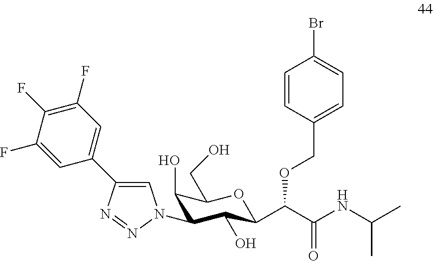

Compound 44: Compound 44 was prepared according to FIG. 3 by using compound 7 instead of compound 28 and using isopropylamine in step e.

¹H NMR (400 MHZ, MEOD) Δ 8.44 (S, 1H), 7.57 (DD, J=8.8, 6.5 HZ, 2H), 7.47-7.41 (M, 2H), 7.30 (D, J=8.3 HZ, 2H), 4.76-4.68 (M, 1H), 4.59-4.47 (M, 2H), 4.47-4.36 (M, 1H), 4.17 (D, J=2.1 HZ, 1H), 4.03 (D, J=3.0 HZ, 1H), 3.93 (P, J=6.5 HZ, 1H), 3.63 (DD, J=9.4, 2.0 HZ, 1H), 3.57 (D, J=1.6 HZ, 3H), 1.04 (T, J=7.2 HZ, 6H). LCMS (ESI): M/Z CALCULATED FOR C₂₆H₂₈BRF₃N₄O₆: 629.4, FOUND 630.1, 632.2 (M+H).

### Example 11

### Synthesis of Compound 45

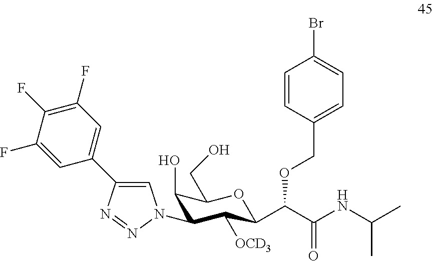

Compound 45: Compound 45 was prepared according to FIG. 1 using deuteromethyl iodide in step 1 and isopropylamine in step p.

¹H NMR (400 MHz, MeOD) δ 8.72 (s, 1H), 7.76-7.64 (m, 2H), 7.60-7.50 (m, 2H), 4.95 (dd, J=10.6, 2.9 Hz, 1H), 4.68 (d, J=11.6 Hz, 1H), 4.56-4.47 (m, 1H), 4.28 (dd, J=10.6, 9.3 Hz, 1H), 4.19 (d, J=2.0 Hz, 1H), 4.16-4.02 (m, 2H), 3.70-3.63 (m, 5H), 1.19 (d, J=6.8 Hz, 6H).

LCMS (ESI): m/z calculated for C₂₇H₂₇D₃BrF₃N₄O₆: 645.1, 647.1, found 646.1, 648.1 (M+H).

### Example 12

### Synthesis of Compound 46

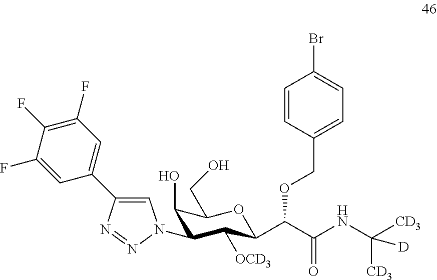

Compound 46: Compound 46 was prepared according to FIG. 1 using deuteromethyl iodide in step 1 and heptadeuteroisopropylamine in step p.

¹H NMR (400 MHz, MeOD) δ 8.72 (s, 1H), 7.76-7.64 (m, 2H), 7.57 (dd, J=8.6, 2.1 Hz, 2H), 7.42 (dd, J=8.6, 2.1 Hz, 2H), 4.95 (dd, J=10.6, 2.9 Hz, 1H), 4.68 (d, J=11.6 Hz, 1H), 4.52 (d, J=11.61 Hz, 1H), 4.28 (dd, J=10.6, 9.4 Hz, 1H), 4.19 (d, J=2.0 Hz, 1H), 4.10 (d, J=2.9 Hz, 1H), 3.77-3.62 (m, 5H). LCMS (ESI): m/z calculated for C₂₇H₂₀D₁₀BrF₃N₄O₆: 652.2, 654.2, found 653.3, 655.3 (M+H).

### Example 13

### Synthesis of Compound 47

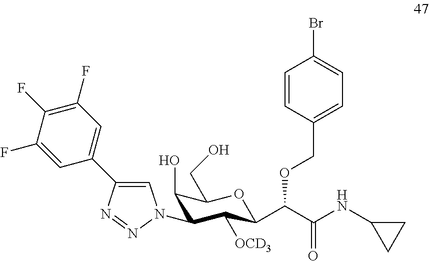

Compound 47: Compound 47 was prepared according to FIG. 1 using deuteromethyl iodide in step 1 and cyclopropylamine in step p.

¹H NMR (400 MHz, MeOD) δ 8.70 (s, 1H), 7.69 (dd, J=8.7, 6.5 Hz, 2H), 7.56 (d, J=8.2 Hz, 2H), 7.41 (d, J=8.2 Hz, 2H), 4.94 (dd, J=10.6, 2.9 Hz, 1H), 4.67 (d, J=11.6 Hz, 1H), 4.50 (d, J=11.6 Hz, 1H), 4.25 (dd, J=10.6, 9.3 Hz, 1H), 4.19 (d, J=2.1 Hz, 1H), 4.08 (d, J=2.9 Hz, 1H), 3.75-3.62 (m, 4H), 2.71 (tt, J=7.4, 3.9 Hz, 1H), 0.77 (td, J=7.0, 4.8 Hz, 2H), 0.60-0.52 (m, 2H). LCMS (ESI): m/z calculated for C₂₇H₂₅D₃BrF₃N₄O₆: 643.1, 645.1, found 644.2, 646.2 (M+H).

### Example 14

### Synthesis of Compound 48

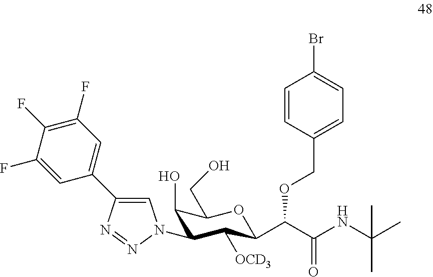

Compound 48: Compound 48 was prepared according to FIG. 1 using deuteromethyl iodide in step 1 and tert-butylamine in step p.

¹H NMR (400 MHz, MeOD) δ 8.73 (s, 1H), 7.76-7.64 (m, 2H), 7.58 (d, J=8.3 Hz, 2H), 7.42 (d, J=8.3 Hz, 2H), 7.04 (s, 1H), 4.96 (dd, J=10.6, 2.9 Hz, 1H), 4.67-4.56 (m, 2H), 4.28 (dd, J=10.6, 9.3 Hz, 1H), 4.11 (dd, J=4.7, 2.5 Hz, 2H), 3.78-3.63 (m, 4H), 1.36 (s, 9H). LCMS (ESI): m/z calculated for C₂₈H₂₉D₃BrF₃N₄O₆: 659.1, 661.1, found 660.3, 662.3 (M+H).

### Example 15

### Synthesis of Compound 49

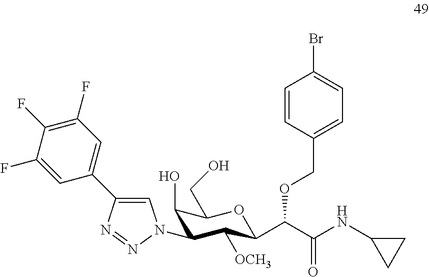

Compound 49: Compound 49 was prepared according to FIG. 1 using cyclopropylamine in step p.

¹H NMR (400 MHz, MeOD) δ 8.70 (s, 1H), 7.76-7.63 (m, 2H), 7.56 (d, J=8.4 Hz, 2H), 7.41 (d, J=8.4 Hz, 2H), 4.95 (dd, J=10.6, 2.9 Hz, 1H), 4.67 (d, J=11.6 Hz, 1H), 4.50 (d, J=11.6 Hz, 1H), 4.26 (t, J=10.0 Hz, 1H), 4.19 (d, J=2.1 Hz, 1H), 4.08 (d, J=2.9 Hz, 1H), 3.76-3.62 (m, 4H), 2.97 (s, 3H), 2.71 (tt, J=7.4, 3.9 Hz, 1H), 0.77 (td, J=7.0, 4.8 Hz, 2H), 0.60-0.52 (m, 2H). LCMS (ESI): m/z calculated for C₂₇H₂₈BrF₃N₄O₆: 640.1, 642.1, found 641.1, 643.1 (M+H).

### Example 16

### Synthesis of Compound 50

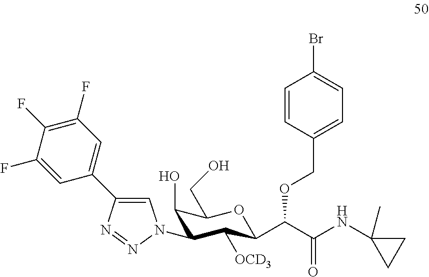

Compound 50: Compound 50 was prepared according to FIG. 1 using deuteromethyl iodide in step 1 and 1-methylcyclopropylamine in step p.

¹H NMR (400 MHz, MeOD) δ 8.70 (s, 1H), 7.76-7.64 (m, 2H), 7.56 (m, J=8.4 Hz, 2H), 7.40 (m, J=8.4 Hz, 2H), 4.94 (dd, J=10.6, 2.9 Hz, 1H), 4.63 (d, J=11.6 Hz, 1H), 4.50 (d, J=11.6 Hz, 1H), 4.26 (dd, J=10.6, 9.3 Hz, 1H), 4.13 (d, J=2.1 Hz, 1H), 3.69-3.61 (m, 2H), 1.37 (s, 3H), 0.81-0.58 (m, 4H). LCMS (ESI): m/z calculated for C₂₈H₂₇D₃BrF₃N₄O₆: 657.1, 659.1, found 658.1, 660.1 (M+H).

### Example 17

### Synthesis of Compound 51

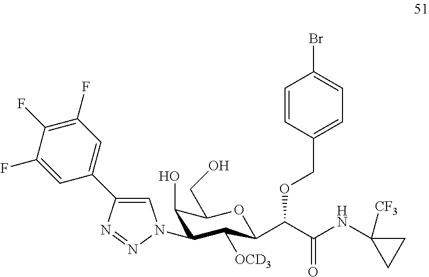

Compound 51: Compound 51 was prepared according to FIG. 1 using deuteromethyl iodide in step 1 and 1-trifluoromethyl cyclopropylamine in step p.

¹H NMR (400 MHz, MeOD) δ 8.58 (s, 1H), 7.58 (dd, J=8.7, 6.5 Hz, 2H), 7.45 (d, J=8.4 Hz, 2H), 7.31 (d, J=8.4 Hz, 2H), 4.82 (dd, J=10.7, 2.9 Hz, 1H), 4.57 (d, J=11.5 Hz, 1H), 4.38 (d, J=11.5 Hz, 1H), 4.20-4.11 (m, 1H), 4.09 (d, J=2.2 Hz, 1H), 3.97 (d, J=2.9 Hz, 1H), 3.61-3.55 (m, 3H), 3.54-3.48 (m, 1H), 1.27-1.13 (m, 2H), 1.07-0.99 (m, 2H). LCMS (ESI): m/z calculated for C₂₈H₂₄D₃BrF₆N₄O₆: 711.1, 713.1, found 712.0, 713.0 (M+H).

### Example 18

### Prophetic Synthesis of Compound 58

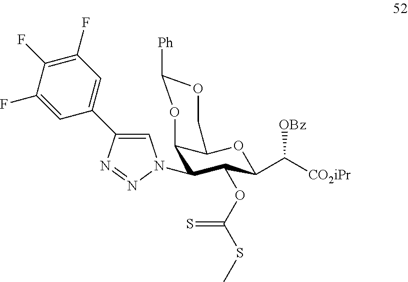

Compound 52: To a solution of compound 13 dissolved in DMSO at room temperature is added DBU (1.2 equivalents) followed by carbon disulfide (10 equivalents). The reaction mixture is stirred for 2 hours after which time iodomethane (2 equivalents) is added. The reaction mixture is stirred until completion. The reaction mixture is diluted with DCM, transferred to a separatory funnel, and washed with H₂O. The organic phase is dried over Na₂SO₄, filtered, and concentrated. The residue is separated by column chromatography to afford compound 52.

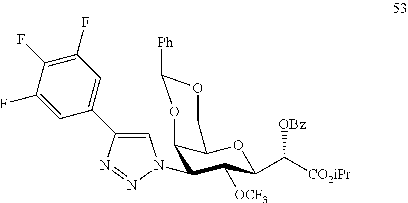

Compound 53: Bis(trifluoroacetoxy)iodobenzene is dissolved in DCM in a polypropylene reaction vessel under argon and cooled to −78° C. To this solution is slowly added 70% HF/pyridine. To this mixture is added compound 52. The temperature is raised to −10° C. and the reaction mixture is stirred at this temperature until completion. The reaction mixture is diluted with ether then quenched by the addition of an ice-cold aqueous NaHSO₃/NaHCO₃/NaOH (pH10) solution. The reaction mixture is transferred to a separatory funnel and the phases separated. The aqueous phase is extracted with ether. The combined organic phases are dried over MgSO₄, filtered, and concentrated. The residue is separated by column chromatography to afford compound 53.

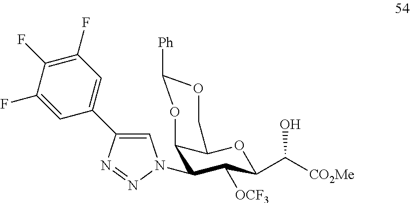

Compound 54: Compound 53 is dissolved in a solution of sodium methoxide in MeOH at room temperature. The reaction mixture is stirred until completion. The pH of the solution is adjusted to 7 by the addition of Amberlite IRC 120H (hydrogen form) resin. The reaction mixture is filtered and concentrated to afford compound 54.

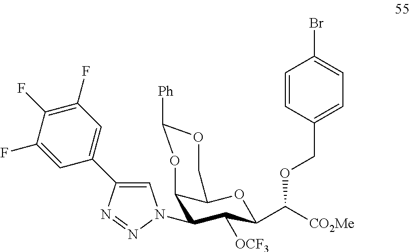

Compound 55: Compound 54 and 4-bromobenzyl bromide are dissolved in anhydrous DMF and cooled on an ice bath. Sodium hydride is added, and the reaction mixture was stirred on the ice bath until completion. The reaction mixture is quenched by the addition of saturated ammonium sulfate solution. The reaction mixture is diluted with DCM, transferred to a separatory funnel, and washed with H₂O. The aqueous phase is separated and extracted with DCM. The combined organic phases are dried over Na₂SO₄, filtered, and concentrated. The residue is separated by column chromatography to afford compound 55.

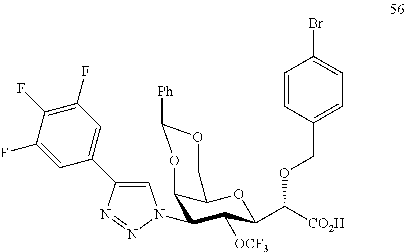

Compound 56: Compound 55 is dissolved in THF and cooled on an ice bath. An aqueous solution of LiOH is added, and the reaction mixture is stirred on the ice bath until completion. The reaction mixture is diluted with EtOAc, transferred to a separatory funnel, and washed with 1M HCL. The aqueous phase is separated and extracted with EtOAc. The combined organic phases are dried over Na₂SO₄, filtered, and concentrated to afford compound 56.

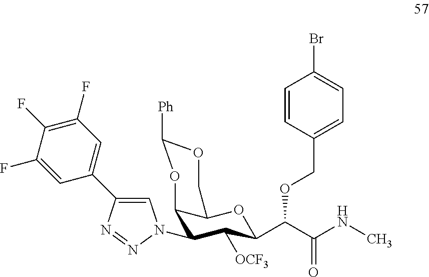

Compound 57: To a solution of compound 56 dissolved in DCM under an argon atmosphere at room temperature is added HATU followed by DIPEA followed by methylamine. The reaction mixture is stirred until completion. The reaction mixture is diluted with DCM, transferred to a separatory funnel, and washed with 0.25 M HCl. The aqueous phase is separated and extracted with DCM. The combined organic phases are washed with saturated NaHCO₃, dried over Na₂SO₄, filtered, and concentrated. The residue is separated by column chromatography to afford compound 57.

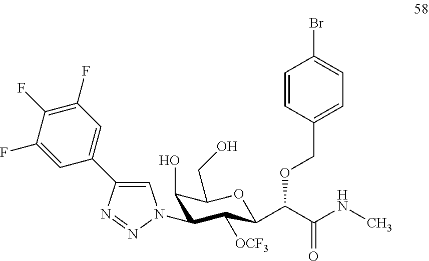

Compound 58: To a solution of compound 57 dissolved in 1,4-dioxane is added HCl. The reaction mixture is stirred at 50° C. until completion. The reaction mixture is cooled to room temperature and diluted with DCM. The reaction mixture is transferred to a separatory funnel and extracted with DCM. The combined organic phases are washed with saturated NaHCO₃, dried over Na₂SO₄, filtered, and concentrated. The residue is separated by column chromatography to afford compound 58.

### Example 19

### Prophetic Synthesis of Compound 59

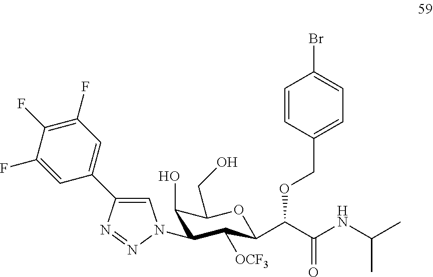

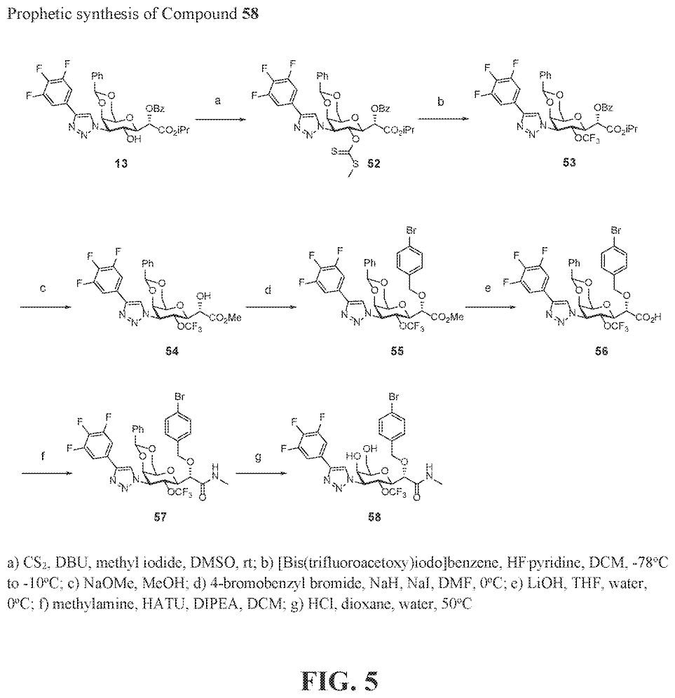

Compound 59: Compound 59 can be prepared according to FIG. 5 using isopropylamine in step f.

### Example 20

### Prophetic Synthesis of Compound 60

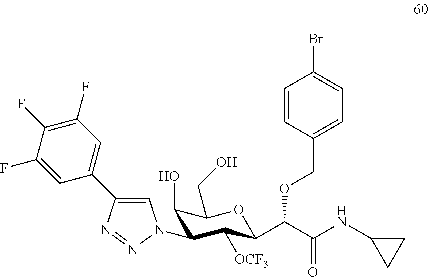

Compound 60: Compound 60 can be prepared according to FIG. 5 using cyclopropylamine in step f.

### Example 21

### Synthesis of Compound 61

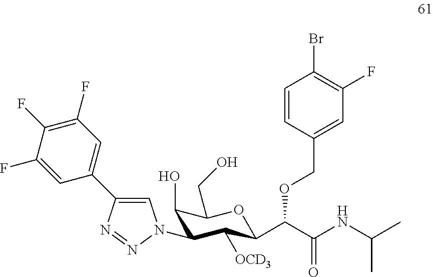

Compound 61: Compound 61 was prepared according to FIG. 1 using deuteromethyl iodide in step 1 and 4-bromo-3-fluorobenzyl bromide in step n.

¹H NMR (400 MHz, MeOD) δ 8.71 (s, 1H), 7.79-7.60 (m, 3H), 7.46 (dd, J=9.6, 1.9 Hz, 1H), 7.23 (dd, J=8.2, 1.9 Hz, 1H), 4.97 (dd, J=10.7, 2.9 Hz, 1H), 4.71 (d, J=12.0 Hz, 1H), 4.55 (d, J=12.0 Hz, 1H), 4.30 (t, J=10.0 Hz, 1H), 4.20 (d, J=2.1 Hz, 1H), 4.16-4.03 (m, 2H), 3.84-3.60 (m, 5H), 1.19 (dd, J=6.6, 4.0 Hz, 6H). LCMS (ESI): m/z calculated for C₂₇H₂₆D₃BrF₄N₄O₆: 663.1, 665.1, found 664.0, 666.0 (M+H).

### Example 22

### Synthesis of Compound 62

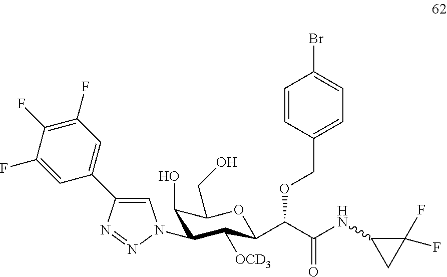

Compound 62: Compound 62 was prepared according to FIG. 1 using deuteromethyl iodide in step 1 and 2,2-difluorocyclopropylamine hydrochloride in step p.

¹H NMR (400 MHz, MeOD) δ 8.58 (s, 1H), 7.58 (dd, J=8.7, 6.5 Hz, 2H), 7.48-7.42 (m, 2H), 7.30 (dd, J=8.2, 5.6 Hz, 2H), 4.87-4.82 (m, 1H), 4.58 (dd, J=11.5, 3.2 Hz, 1H), 4.37 (d, J=11.5 Hz, 1H), 4.22-4.11 (m, 2H), 4.02-3.94 (m, 1H), 3.58 (dddd, J=12.6, 10.3, 7.1, 2.9 Hz, 5H), 1.75 (ddd, J=9.8, 6.6, 2.9 Hz, 1H), 1.45 (ddt, J=14.5, 10.7, 5.4 Hz, 1H), 0.85-0.74 (m, 1H). LCMS (ESI): m/z calculated for C₂₇H₂₃D₃BrF₅N₄O₆: 679.1, 681.1, found 680.0, 682.0 (M+H).

### Example 23

### SYNTHESIS of Compound 63

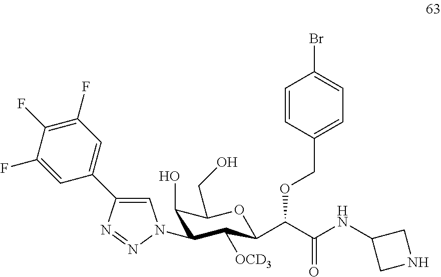

Compound 63: Compound 63 was prepared according to FIG. 1 using deuteromethyl iodide in step 1 and 3-amino-1-N-Boc-azetidine in step p.

¹H NMR (400 MHz, Methanol-d₄) δ 8.70 (s, 1H), 7.76-7.65 (m, 2H), 7.59 (d, J=8.2 Hz, 2H), 7.45 (d, J=8.2 Hz, 2H), 4.96 (dd, J=10.7, 2.9 Hz, 1H), 4.76 (d, J=11.7 Hz, 1H), 4.71 (t, J=7.6 Hz, 1H), 4.56 (d, J=11.6 Hz, 1H), 4.36-4.25 (m, 6H), 4.06 (d, J=2.9 Hz, 1H), 3.78-3.65 (m, 4H), 1.40-1.21 (m, 1H). LCMS (ESI): m/z calculated for C₂₇H₂₆D₃BrF₃N₅O₆: 658.1, 660.1, found 659.0, 661.0 (M+H).

### Example 24

### Synthesis of Compound 64

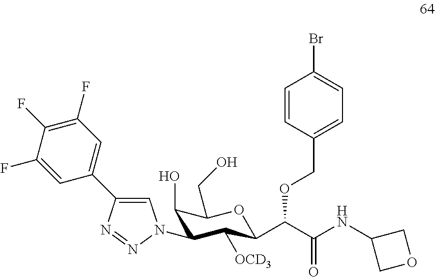

Compound 64: Compound 64 was prepared according to FIG. 1 using deuteromethyl iodide in step 1 and 3-amino-oxetane in step p.

¹H NMR (400 MHz, MeOD) δ 8.71 (s, 1H), 7.70 (dd, J=8.7, 6.5 Hz, 2H), 7.58 (t, J=8.2 Hz, 1H), 7.32-7.25 (m, 2H), 4.96 (dd, J=10.6, 2.9 Hz, 1H), 4.75 (d, J=11.3 Hz, 1H), 4.59 (d, J=11.3 Hz, 1H), 4.27 (t, J=10.0 Hz, 1H), 4.21 (d, J=2.0 Hz, 1H), 4.09 (q, J=5.3 Hz, 2H), 3.74 (dd, J=9.3, 2.2 Hz, 1H), 3.68 (q, J=4.5 Hz, 3H), 1.21 (dd, J=6.6, 2.8 Hz, 6H). LCMS (ESI): m/z calculated for C₂₇H₂₅D₃BrF₃N₅O₇: 659.1, 661.1, found 660.2, 662.0 (M+H).

### Example 25

### Synthesis of Compound 65

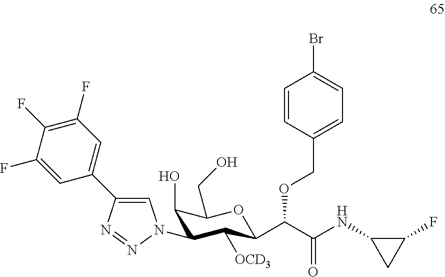

Compound 65: Compound 65 was prepared according to FIG. 1 using deuteromethyl iodide in step 1 and (R,S)-1-fluoro-2-aminocyclopropane in step p.

¹H NMR (400 MHz, MeOD) δ 8.71 (s, 1H), 7.76-7.64 (m, 2H), 7.59-7.52 (m, 2H), 7.41 (d, J=8.2 Hz, 2H), 4.95 (dd, J=10.6, 3.0 Hz, 1H), 4.81 (td, J=5.6, 3.1 Hz, 1H), 4.69 (d, J=11.3 Hz, 1H), 4.64 (dt, J=5.7, 2.8 Hz, 1H), 4.50 (d, J=11.4 Hz, 1H), 4.32-4.21 (m, 2H), 4.09 (d, J=2.9 Hz, 1H), 3.74 (dd, J=9.4, 2.2 Hz, 1H), 3.72-3.63 (m, 3H), 2.71 (dt, J=9.0, 5.4 Hz, 1H), 1.19 (dtd, J=14.8, 8.5, 6.1 Hz, 1H), 1.04 (dddd, J=24.9, 8.5, 5.7, 3.1 Hz, 1H). LCMS (ESI): m/z calculated for C₂₇H₂₄D₃BrF₄N₄O₆: 661.1, 663.1, found 662.2, 664.0 (M+H).

### Example 26

### Synthesis of Compound 66

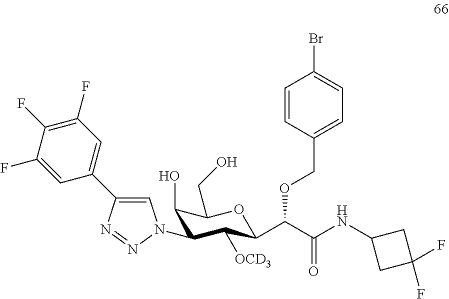

Compound 66: Compound 66 was prepared according to FIG. 1 using deuteromethyl iodide in step 1 and 1,1-difluoro-3-aminocyclobutane in step p.

¹H NMR (400 MHz, DMSO) δ 8.92 (s, 1H), 8.36 (d, J=7.5 Hz, 1H), 7.91 (dd, J=9.1, 6.6 Hz, 2H), 7.63-7.54 (m, 2H), 7.44 (d, J=8.4 Hz, 2H), 5.40 (d, J=5.9 Hz, 1H), 5.01 (dd, J=10.6, 2.9 Hz, 1H), 4.64 (d, J=11.6 Hz, 1H), 4.56 (t, J=5.5 Hz, 1H), 4.41 (d, J=11.6 Hz, 1H), 4.15 (dd, J=14.8, 7.6 Hz, 1H), 4.12-4.06 (m, 1H), 4.04 (d, J=2.0 Hz, 1H), 3.89 (dd, J=5.8, 2.9 Hz, 1H), 3.69 (dd, J=9.4, 2.1 Hz, 1H), 3.58 (t, J=6.4 Hz, 1H), 3.49 (dt, J=13.0, 6.6 Hz, 1H), 3.40 (dt, J=11.0, 5.6 Hz, 1H), 2.86 (ddt, J=18.8, 12.8, 6.4 Hz, 4H), 1.23 (s, 1H). LCMS (ESI): m/z calculated for C₂₈H₂₅D₃BrF₅N₄O₆: 693.1, 695.1, found 694.1, 696.1 (M+H).

### Example 27

### Synthesis of Compound 67

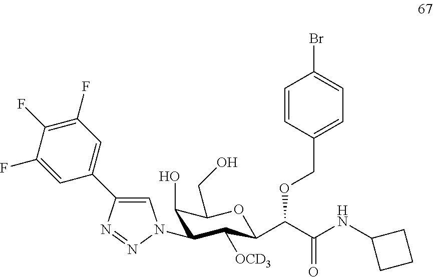

Compound 67: Compound 67 was prepared according to FIG. 1 using deuteromethyl iodide in step 1 and aminocyclobutane in step p.

¹H NMR (400 MHz, MeOD) δ 8.71 (s, 1H), 7.76-7.65 (m, 2H), 7.61-7.53 (m, 2H), 7.49-7.39 (m, 2H), 4.94 (dd, J=10.6, 2.9 Hz, 1H), 4.68 (d, J=11.6 Hz, 1H), 4.52 (d, J=11.6 Hz, 1H), 4.36 (p, J=8.3 Hz, 1H), 4.27 (t, J=9.9 Hz, 1H), 4.18 (d, J=2.0 Hz, 1H), 4.10 (d, J=2.9 Hz, 1H), 3.76-3.60 (m, 4H), 2.30 (ddq, J=11.3, 7.3, 4.0 Hz, 2H), 2.04 (p, J=9.5, 8.9 Hz, 2H), 1.76 (tt, J=10.6, 6.5 Hz, 2H). LCMS (ESI): m/z calculated for C₂₈H₂₇D₃BrF₃N₄O₆: 657.2, 659.2, found 658.1, 660.1 (M+H).

### Example 28

### Synthesis of Compound 68

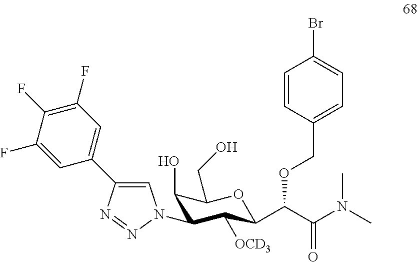

Compound 68: Compound 68 was prepared according to FIG. 1 using deuteromethyl iodide in step 1 and dimethylamine in step p.

¹H NMR (400 MHz, MeOD) δ 8.73 (s, 1H), 7.75-7.63 (m, 2H), 7.56 (d, J=8.2 Hz, 2H), 7.41 (d, J=8.2 Hz, 2H), 4.94 (dd, J=10.6, 2.9 Hz, 1H), 4.70-4.63 (m, 2H), 4.45 (d, J=11.3 Hz, 1H), 4.26 (dd, J=10.6, 9.2 Hz, 1H), 4.06 (d, J=2.9 Hz, 1H), 3.86-3.64 (m, 4H), 3.33 (h, J=1.4 Hz, 2H), 3.21 (s, 3H), 3.02 (s, 3H), 2.97 (s, 6H). LCMS (ESI): m/z calculated for C₂₆H₂₅D₃BrF₃N₄O₆: 631.1, 633.1, found 632.2, 634.2 (M+H).

### Example 29

### Synthesis of Compound 69

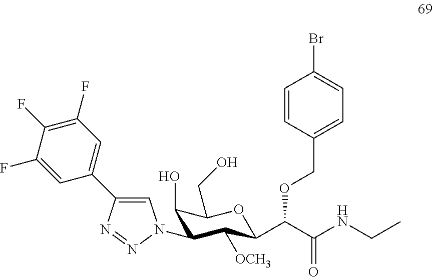

Compound 69: Compound 69 was prepared according to FIG. 1 using ethylamine in step p.

¹H NMR (400 MHz, Methanol-d4) δ 8.70 (s, 1H), 7.70 (dd, J=8.7, 6.5 Hz, 2H), 7.57 (d, J=8.3 Hz, 2H), 7.44 (d, 0.1=8.2 Hz, 2H), 4.95 (dd, 1=10.7, 3.0 Hz, 1H), 4.72 (d, 0.1=11.5 Hz, 1H), 4.51 (d, J=11.4 Hz, 1H), 4.27 (d, J=8.0 Hz, 1H), 4.20 (d, J=4.0 Hz, 1H), 4.09 (broad d, J=2.8 Hz, 1H), 3.77-3.62 (m, 4H), 3.31-3.10 (m, 2H), 2.97 (s, 3H), 1.16-1.10 (two t, J=8.0 Hz, 3H). LCMS (ESI): m/z calculated for C₂₆H₂₈BrF₃N₄O₆: 628.1, 630.1, found 639.1, 631.1 (M+H).

### Example 30

### Synthesis of Compound 70

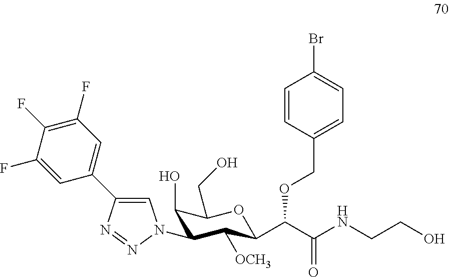

Compound 70: Compound 70 was prepared according to FIG. 1 using 2-aminoethanol in step p.

¹H NMR (400 MHz, Methanol-d4) δ 8.70 (s, 1H), 7.70 (dd, J=8.7, 6.6 Hz, 2H), 7.61-7.53 (m, 2H), 7.45 (d, J=8.3 Hz, 2H), 4.96 (dd, J=10.6, 2.9 Hz, 1H), 4.75 (d, J=11.5 Hz, 1H), 4.52 (d, J=11.5 Hz, 1H), 4.28 (t, J=11.5 Hz, 1H), 4.24 (d, J=3.0 Hz, 1H), 4.06 (d, J=2.9 Hz, 1H), 3.80-3.70 (m, 2H), 3.69-3.60 (m, 4H), 3.56-3.45 (m, 1H),), 3.41-3.34 (m, 1H), 2.99 (s, 3H). LCMS (ESI): m/z calculated for C₂₆H₂₈BrF₃N₄O₇: 644.1, 646.1, found 645.2, 647.2 (M+H).

### Example 31

### Synthesis of Compound 71

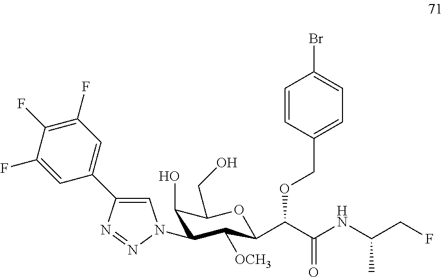

Compound 71: Compound 71 was prepared according to FIG. 1 using (S)-1-fluoro-2-aminopropane in step p.

¹H NMR (400 MHz, Methanol-d4) δ 8.60 (s, 1H), 7.59 (dd, J=8.6, 6.5 Hz, 2H), 7.45 (d, J=8.1 Hz, 2H), 7.31 (d, J=8.1 Hz, 2H), 4.83 (dd, J=10.6, 2.9 Hz, 1H), 4.59 (d, J=11.3 Hz, 1H), 4.43-4.31 (m, 2H), 4.30-4.09 (m, 4H), 3.98 (d, J=2.9 Hz, 1H), 3.66-3.51 (m, 4H), 2.89 (s, 3H), 1.11 (dd, J=10.6, 2.9 Hz, 2H). LCMS (ESI): m/z calculated for C₂₇H₂₉BrF₃N₄O₆: 660.1, 662.1, found 661.1, 663.1 (M+H).

### Example 32

### Synthesis of Compound 72

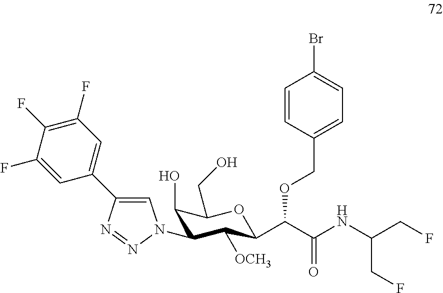

Compound 72: Compound 72 was prepared according to FIG. 1 using 1,3-difluoro-2-aminopropane in step p.

¹H NMR (400 MHz, Methanol-d4) δ 8.60 (s, 1H), 7.58 (dd, J=8.7, 6.5 Hz, 2H), 7.45 (d, J=8.3 Hz, 2H), 7.31 (d, J=8.4 Hz, 2H), 4.84 (dd, J=10.6, 3.0 Hz, 1H), 4.60 (d, J=12.0 Hz, 1H), 4.56-4.46 (m, 2H), 4.48-4.27 (m, 4H), 4.23-4.13 (m, 2H), 3.97 (d, J=2.9 Hz, 1H), 3.66-3.50 (m, 4H), 2.89 (s, 3H). LCMS (ESI): m/z calculated for C₂₇H₂₈BrF₅N₄O₆: 678.1, 680.1, found 679.2, 681.2 (M+H).

### Example 33

### Synthesis of Compound 73

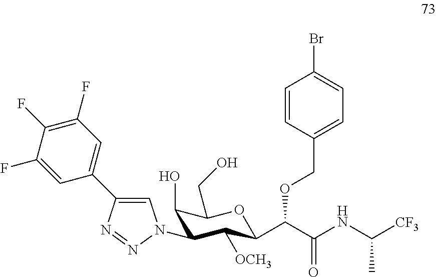

Compound 73: Compound 73 was prepared according to FIG. 1 using (S)-1,1,1-trifluoro-2-aminopropane in step p.

¹H NMR (400 MHz, Methanol-d₄) δ 8.60 (d, J=3.2 Hz, 1H), 7.58 (dd, J=8.7, 6.5 Hz, 2H), 7.45 (d, J=8.3 Hz, 2H), 7.30 (dd, J=8.7, 2.7 Hz, 2H), 4.83 (dd, J=10.6, 3.0 Hz, 1H), 4.72-4.60 (m, 1H), 4.57 (d, J=11.3 Hz, 1H), 4.36 (d, J=11.3 Hz, 1H), 4.23-4.13 (m, 2H), 3.98 (d, J=2.9 Hz, 1H), 3.64-3.51 (m, 4H), 2.88 (s, 3H), 1.28 (d, J=7.1 Hz, 3H). LCMS (ESI): m/z calculated for C₂₇H₂₇BrF₆N₄O₆: 696.1, 698.1, found 697.3, 699.3 (M+H).

### Example 34

### Synthesis of Compound 74

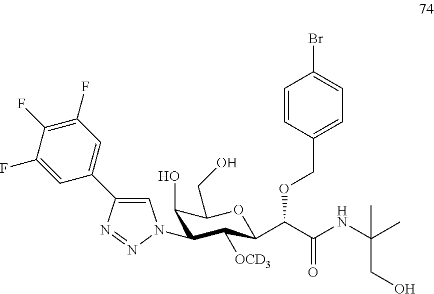

Compound 74: Compound 74 was prepared according to FIG. 1 using deuteromethyl iodide in step 1 and 2-amino-2-methyl-1-propanol in step p.

¹H NMR (400 MHz, DMSO-d6) δ 8.94 (s, 1H), 7.91 (dd, J=9.1, 6.7 Hz, 2H), 7.71-7.57 (m, 2H), 7.48-7.41 (m, 2H), 7.37 (t, J=6.1 Hz, 1H), 5.39 (d, J=5.9 Hz, 1H), 5.05 (dd, J=10.7, 2.9 Hz, 1H), 4.67-4.61 (m, 2H), 4.57 (dd, J=6.3, 4.9 Hz, 1H), 4.46 (d, J=11.4 Hz, 1H), 4.15-4.06 (m, 2H), 3.92 (dd, J=6.1, 3.0 Hz, 1H), 3.74 (dd, J=9.4, 1.9 Hz, 1H), 3.59 (t, J=6.6 Hz, 1H), 3.54-3.36 (m, 3H), 3.19 (dd, J=13.0, 6.5 Hz, 1H), 3.09 (dd, J=13.1, 5.7 Hz, 1H), 1.10 (s, 3H), 1.08 (s, 3H). LCMS (ESI): m/z calculated for C₂₈H₂₉D₃BrF₃N₄O₇: 675.2, 677.2, found 676.1, 678.1 (M+H).

### Example 35

### Synthesis of Compound 75

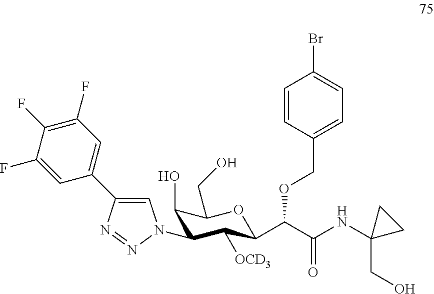

Compound 75: Compound 75 was prepared according to FIG. 1 using deuteromethyl iodide in step 1 and 1-amino-1-hydroxymethylcyclopropane in step p.

¹H NMR (400 MHz, Methanol-d4) δ 8.71 (s, 1H), 7.70 (dd, J=8.7, 6.6 Hz, 2H), 7.58 (d, J=8.3 Hz, 2H), 7.46 (d, J=8.2 Hz, 2H), 4.96 (dd, J=10.6, 2.9 Hz, 1H), 4.77 (d, J=11.4 Hz, 1H), 4.54 (d, J=11.4 Hz, 1H), 4.35-4.23 (m, 2H), 4.08 (d, J=2.9 Hz, 1H), 3.82-3.61 (m, 4H), 3.54-3.37 (m, 2H), 0.81-0.55 (m, 4H). LCMS (ESI): m/z calculated for C₂₈H₂₇D₃BrF₃N₄O₇: 673.1, 675.1, found 674.1, 676.1 (M+H).

### Example 36

### Synthesis of Compound 76

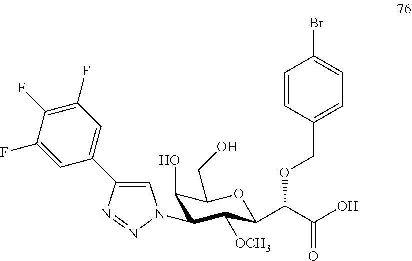

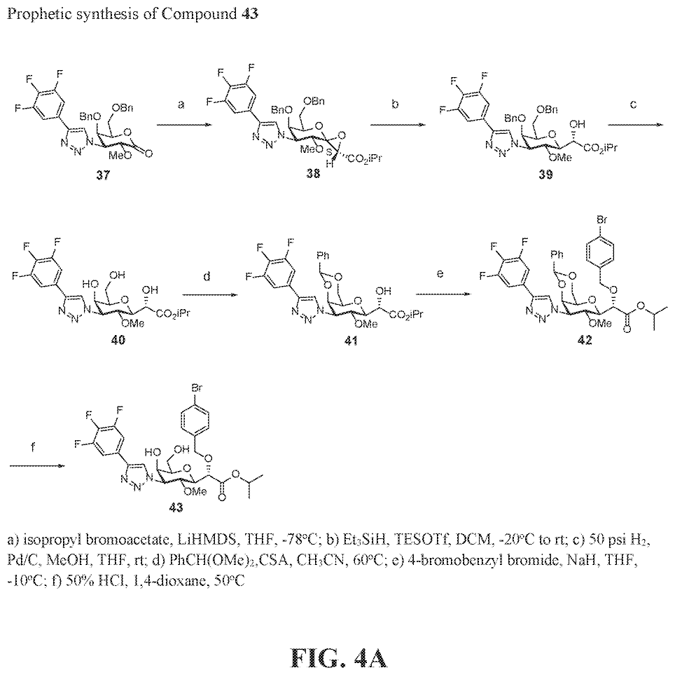

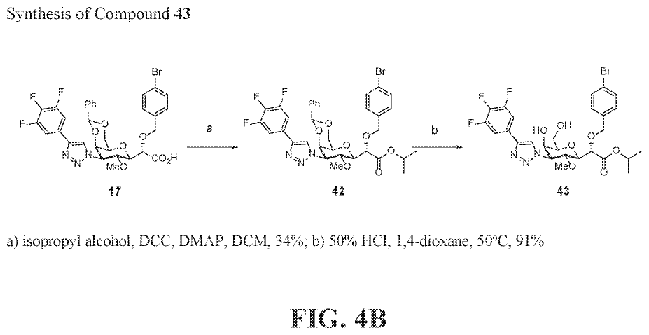

Compound 76: Compound 76 was prepared according to FIG. 4B by omitting step a.

¹H NMR (400 MHz, Methanol-d4) δ 8.66 (s, 1H), 7.67 (dd, J=8.7, 6.6 Hz, 2H), 7.52 (d, J=8.2 Hz, 2H), 7.45 (d, J=8.2 Hz, 2H), 4.90 (m, 1H, overlapped with solvent), 4.85 (d, 1H, overlapped with solvent), 4.35 (d, J=11.2 Hz, 1H), 4.20 (t, J=10.1 Hz, 1H), 4.13 (d, J=1.9 Hz, 1H), 4.00 (d, J=2.9 Hz, 1H), 3.86 (dd, J=9.4, 2.0 Hz, 1H), 3.77 (dd, J=10.6, 5.3 Hz, 1H), 3.70-3.58 (m, 2H), 3.01 (s, 3H). LCMS (ESI): m/z calculated for C₂₄H₂₃D₃BrF₃N₃O₇: 601.1, 603.1, found 602.1, 604.1 (M+H).

### Example 37

### Prophetic Synthesis of Compound 77

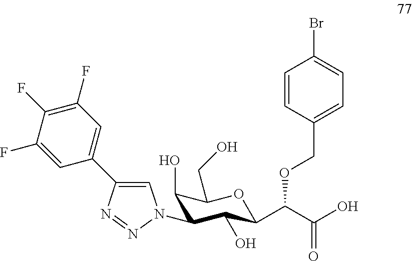

Compound 77: Compound 77 can be prepared according to FIG. 1 by omitting step p.

### Example 38

### Prophetic Synthesis of Compound 78

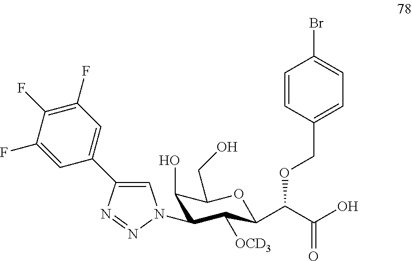

Compound 78: Compound 78 can be prepared according to FIG. 1 using deuteromethyliodide in step 1 and omitting step p.

### Example 39

### Prophetic Synthesis of Compound 79

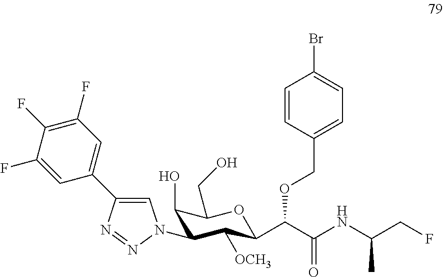

Compound 79: Compound 79 can be prepared according to FIG. 1 using (R)-1-fluoro-2-aminopropane in step p.

### Example 40

### Prophetic Synthesis of Compound 80

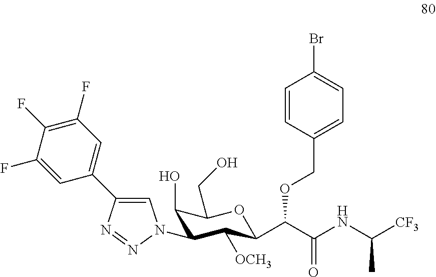

Compound 80: Compound 80 can be prepared according to FIG. 1 using (R)-1,1,1-trifluoro-2-aminopropane in step p.

### Example 41

### Prophetic Synthesis of Compound 81

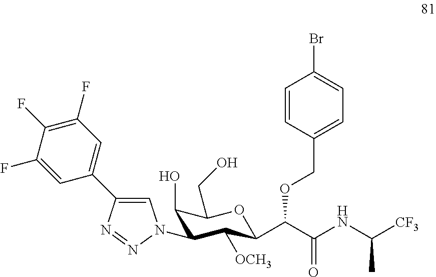

Compound 81: Compound 81 can be prepared according to FIG. 1 using deuteromethyl iodide in step 1 and (R)-1,1,1-trifluoro-2-aminopropane in step p.

### Example 42

### Prophetic Synthesis of Compound 82

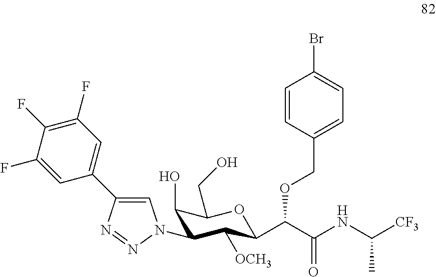

Compound 82: Compound 82 can be prepared according to FIG. 1 using deuteromethyl iodide in step 1 and (S)-1,1,1-trifluoro-2-aminopropane in step p.

### Example 43

### Synthesis of Comparator Compound 83

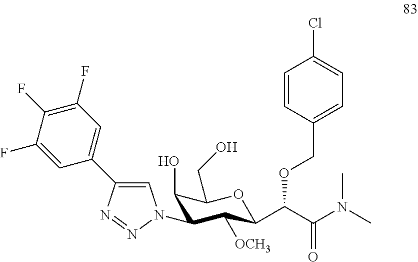

Compound 83: Compound 83 can be prepared according to FIG. 1 using 4-chlorobenzyl bromide in step n and dimethylamine in step p.

¹H NMR (400 MHz, DMSO-d₆) δ 8.99 (s, 1H), 7.89 (dd, J=9.1, 6.7 Hz, 2H), 7.52-7.39 (m, 4H), 5.43 (d, J=6.0 Hz, 1H), 5.03 (dd, J=10.6, 2.9 Hz, 1H), 4.63-4.58 (m, 1H), 4.55 (d, J=11.2 Hz, 1H), 4.43 (d, J=11.2 Hz, 1H), 4.39 (d, J=2.7 Hz, 1H), 4.10 (t, J=10.0 Hz, 1H), 3.92-3.85 (m, 1H), 3.75 (dd, J=9.3, 2.7 Hz, 1H), 3.64 (t, J=6.4 Hz, 1H), 3.54-3.46 (m, 1H), 3.42 (dt, J=10.7, 5.5 Hz, 1H), 3.14 (s, 3H), 2.98 (s, 3H), 2.87 (s, 3H). LCMS (ESI): m/z calculated for C₂₆H₂₈ClF₃N₄O₆: 584.9, found 607.1 (M+Na).

### Example 44

### Synthesis of Comparator Compound 84

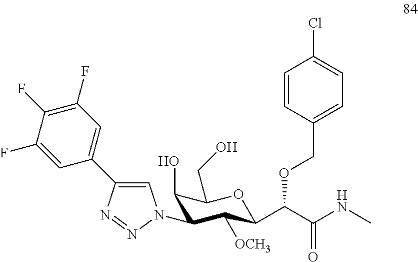

Compound 84: Compound 84 can be prepared according to FIG. 1 using 4-chlorobenzyl bromide in step n.

¹H NMR (400 MHz, Methanol-d4) δ 8.57 (s, 1H), 7.58 (dd, J=8.6, 6.6 Hz, 2H), 7.38 (d, J=8.1 Hz, 2H), 7.30 (d, J=8.2 Hz, 2H), 4.83 (dd, J=10.5, 2.9 Hz, 1H), 4.62 (d, J=11.5 Hz, 1H), 4.39 (d, J=11.6 Hz, 1H), 4.20-4.08 (m, 2H), 3.96 (d, J=2.9 Hz, 1H), 3.69-3.44 (m, 4H), 2.84 (s, 3H), 2.71 (s, 3H). LCMS (ESI): m/z calculated for C₂₅H₂₆ClF₃N₄O₆: 570.1, found 593.1 (M+Na).

### Example 45

### Synthesis of Comparator Compound 85

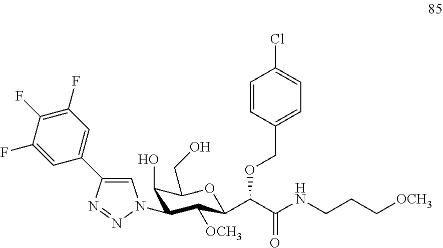

Compound 85: Compound 85 can be prepared according to FIG. 1 using 4-chlorobenzyl bromide in step n and 3-methoxypropylamine in step p.

¹H NMR (400 MHz, MeOD) δ 8.58 (d, J=1.3 Hz, 1H), 7.58 (dd, J=8.6, 6.5 Hz, 2H), 7.39 (d, J=8.1 Hz, 2H), 7.34-7.27 (m, 2H), 4.84 (dd, J=10.5, 2.8 Hz, 1H), 4.62 (d, J=11.6 Hz, 1H), 4.42 (d, J=11.6 Hz, 1H), 4.16 (t, J=10.0 Hz, 1H), 4.10 (d, J=2.0 Hz, 1H), 3.98 (t, J=1.9 Hz, 1H), 3.63 (dd, J=9.4, 2.0 Hz, 1H), 3.61-3.51 (m, 3H), 3.36 (dd, J=6.7, 5.4 Hz, 2H), 3.26 (t, J=6.7 Hz, 2H), 3.21 (d, J=1.5 Hz, 5H), 2.86 (d, J=1.4 Hz, 3H), 1.69 (p, J=6.4 Hz, 2H). LCMS (ESI): m/z calculated for C₂₈H₃₂ClF₃N₄O₇: 628.2, found 629.3 (M+H).

### Example 46

### Synthesis of Comparator Compound 86

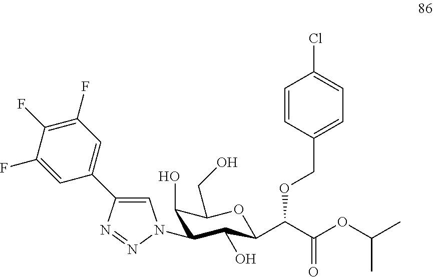

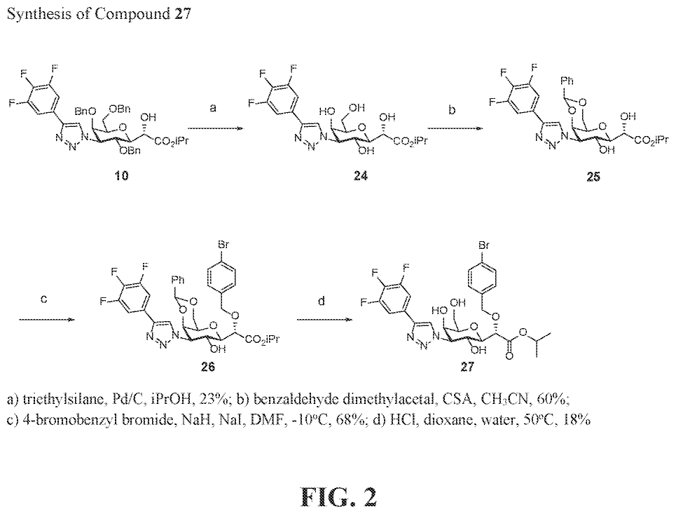

Compound 86: Compound 86 can be prepared according to FIG. 2 using 4-chlorobenzyl bromide in step c.

¹H NMR (400 MHz, DMSO) δ 8.79 (s, 1H), 7.90 (dd, J=9.1, 6.7 Hz, 2H), 7.52-7.41 (m, 4H), 5.46 (d, J=7.6 Hz, 1H), 5.27 (d, J=6.0 Hz, 1H), 5.03 (p, J=6.2 Hz, 1H), 4.84 (dd, J=10.7, 3.0 Hz, 1H), 4.66 (d, J=11.4 Hz, 1H), 4.59 (t, J=5.5 Hz, 1H), 4.53 (d, J=11.3 Hz, 1H), 4.39 (d, J=2.5 Hz, 1H), 4.38-4.30 (m, 1H), 3.93 (t, J=4.5 Hz, 1H), 3.79 (dd, J=9.3, 2.5 Hz, 1H), 3.57 (t, J=6.3 Hz, 1H), 3.48 (dt, J=12.7, 6.3 Hz, 1H), 3.38 (dt, J=10.9, 5.6 Hz, 1H), 1.25 (dd, J=6.3, 4.8 Hz, 7H).). LCMS (ESI): m/z calculated for C₂₆H₂₇ClF₃N₃O₇: 585.2, found 586.1 (M+H).

### Example 47

### Synthesis of Comparator Compound 87

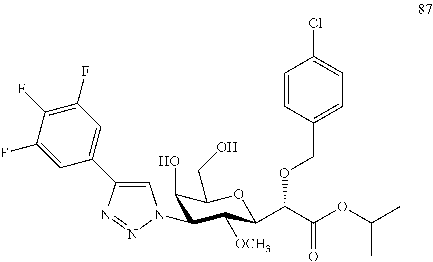

Compound 87: Compound 87 can be prepared according to FIG. 4B using the 4-chlorobenzyl analog of compound 17 as the starting material.

¹H NMR (400 MHz, MeOD) δ 8.72 (s, 1H), 7.69 (dd, J=8.8, 6.5 Hz, 2H), 7.57-7.47 (m, 2H), 7.47-7.28 (m, 2H), 5.19 (p, J=6.2 Hz, 1H), 4.95 (dd, J=10.6, 2.9 Hz, 1H), 4.84 (s, 1H), 4.51 (d, J=11.0 Hz, 1H), 4.45 (d, J=2.5 Hz, 1H), 4.26 (dd, J=10.6, 9.4 Hz, 1H), 4.08 (d, J=2.9 Hz, 1H), 3.91 (dd, J=9.4, 2.5 Hz, 1H), 3.74-3.62 (m, 3H), 3.06 (s, 3H), 1.41-1.30 (m, 6H). LCMS (ESI): m/z calculated for C₂₇H₂₉ClF₃N₃O₇: 599.1, found 600.1 (M+1).

### Example 48

### Synthesis of Comparator Compound 88

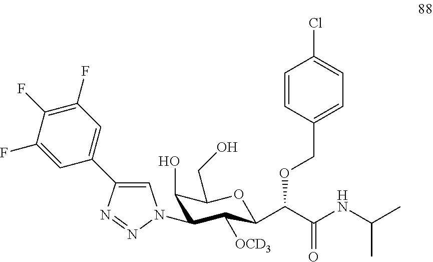

Compound 88: Compound 88 can be prepared according to FIG. 1 using deuteromethyl iodide in step 1, 4-chlorobenzyl bromide in step n, and isopropylamine in step p.

¹H NMR (400 MHz, MeOD) δ 8.71 (s, 1H), 7.70 (dd, J=8.8, 6.6 Hz, 2H), 7.49 (d, J=8.4 Hz, 2H), 7.42 (d, J=8.5 Hz, 2H), 4.95 (dd, J=10.6, 2.9 Hz, 1H), 4.70 (d, J=11.5 Hz, 1H), 4.54 (d, J=11.5 Hz, 1H), 4.32-4.24 (m, 1H), 4.19 (d, J=2.1 Hz, 1H), 4.15-4.01 (m, 2H), 3.78-3.60 (m, 4H), 1.19 (d, J=6.6 Hz, 6H). LCMS (ESI): m/z calculated for C₂₇H₂₇D₃ClF₃N₄O₆: 602.02 found 602.1 (M+H)⁺.

### Example 49

### Synthesis of Comparator Compound 89

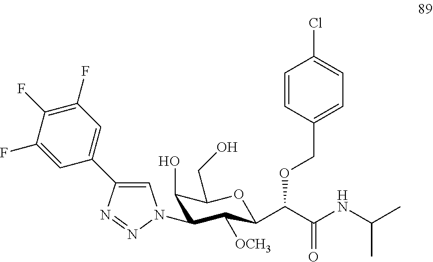

Compound 89: Compound 89 can be prepared according to FIG. 1 using 4-chlorobenzyl bromide in step n and isopropylamine in step p.

¹H NMR (400 MHz, MeOD) δ 8.71 (s, 1H), 7.70 (dd, J=8.8, 6.6 Hz, 2H), 7.66 (d, J=9.1 Hz, 1H), 7.53-7.46 (m, 2H), 7.46-7.38 (m, 2H), 4.95 (dd, J=10.6, 2.9 Hz, 1H), 4.70 (d, J=11.6 Hz, 1H), 4.54 (d, J=11.5 Hz, 1H), 4.28 (dd, J=10.6, 9.4 Hz, 1H), 4.19 (d, J=2.1 Hz, 1H), 4.15-4.03 (m, 2H), 3.79-3.62 (m, 4H), 3.00 (s, 3H), 1.19 (d, J=6.6 Hz, 6H). LCMS (ESI): m/z calculated for C₂₇H₃₀ClF₃N₄O₆: 599.00 found 599.3 (M+H)*.

### Example 50

### Synthesis of Comparator Compound 90

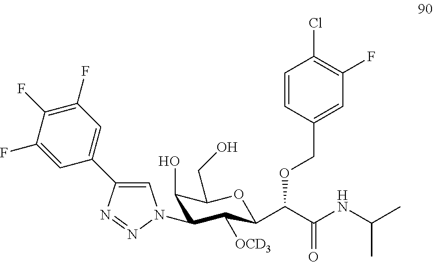

Compound 90: Compound 90 can be prepared according to FIG. 1 using deuteromethyl iodide in step 1, 4-chloro-3-fluorobenzyl bromide in step n, and isopropylamine in step p.

¹H NMR (400 MHz, MeOD) δ 8.59 (s, 1H), 7.57 (dd, J=8.7, 6.5 Hz, 2H), 7.47-7.32 (m, 2H), 7.17 (dd, J=8.3, 1.9 Hz, 1H), 4.85 (dd, J=10.6, 2.9 Hz, 1H), 4.59 (d, J=12.0 Hz, 1H), 4.44 (d, J=12.0 Hz, 1H), 4.18 (dd, J=10.5, 9.4 Hz, 1H), 4.08 (d, J=2.1 Hz, 1H), 4.02-3.90 (m, 2H), 3.71-3.52 (m, 4H), 1.07 (dd, J=6.7, 3.6 Hz, 6H). LCMS (ESI): m/z calculated for C₂₇H₂₆D₃ClF₄N₄O₆: 619.1, found 620.0 (M+H).

### Example 51

### Galectin-3 Activity—ELISA Assay

Galectin-3 antagonists were evaluated for their ability to inhibit binding of galectin-3 to a Galβ1-3GlcNAc carbohydrate structure. The detailed protocol was as follows. A 1 μg/mL suspension of a Galβ1-3GlcNAcβ1-3Galβ1-4GlcNAcβ-PAA-biotin polymer (Glycotech, catalog number 01-096) was prepared. A 100 μL aliquot of the polymer was added to the wells of a 96-well streptavidin-coated plate (R&D Systems, catalog number CP004). A 100 μL aliquot of 1× Tris Buffered Saline (TBS, Sigma, catalog number T5912-10X) was added to control wells. The polymer was allowed to bind to the streptavidin-coated wells for 1.5 hours at room temperature. The contents of the wells were discarded, and 200 μL of 1×TBS containing 1% bovine serum albumin (BSA) was added to each well as a blocking reagent and the plate was kept at room temperature for 30 minutes. The wells were washed three times with 1×TBS containing 0.1% BSA. A serial dilution of test compounds was prepared in a separate V-bottom plate (Corning, catalog number 3897). A 75 μL aliquot of the highest concentration of the compound to be tested was added to the first well in a column of the V-bottom plate, then 15 μL were serially transferred into 60 μL 1×TBS through the remaining wells in the column to generate a 1 to 5 serial dilution. A 60 μL aliquot of 2 μg/mL galectin-3 (IBL, catalog number IBATGP0414) was added to each well in the V-bottom plate. A 100 μL aliquot of the galectin-3 test compound mixture was transferred from the V-bottom plate into the assay plate containing the Galβ1-3GlcNAc polymer. Four sets of control wells in the assay plate were prepared in duplicate containing 1) both Galβ1-3GlcNAc polymer and galectin-3, 2) neither the polymer nor galectin-3, 3) galectin-3 only, no polymer, or 4) polymer only, no galectin-3. The plate was gently rocked for 1.5 hours at room temperature. The wells were washed four times with TBS/0.1% BSA. A 100 μL aliquot of anti-galectin-3 antibody conjugated to horse radish peroxidase (R&D Systems, from DGAL30 kit) was added to each well, and the plate was kept at room temperature for 1 hour. The wells were washed four times with TBS/0.1% BSA. A 100 μL aliquot of TMB substrate solution was added to each well. The TMB substrate solution was prepared by making a 1:1 mixture of TMB Peroxidase Substrate (KPL, catalog number 5120-0048) and Peroxidase Substrate Solution B (KPL, catalog number 5120-0037). The plate was kept at room temperature for 10 to 20 minutes. The color development was stopped by adding 100 uL 10% phosphoric acid (RICCA Chemical Co., catalog number 5850-16). The absorbance at 450 nm (A₄₅₀) was measured using a FlexStation 3 plate reader (Molecular Devices). Plots of A₄₅₀ versus test compound concentration and IC₅₀ determinations were made using GraphPad Prism 6. The number of assays run (n) for each compound is recorded in the following table. For compounds assayed more than once, the average of all assays, standard deviation between the IC50 values obtained.

### Galectin-3 Antagonist Activity (ELISA Assay)

| Compound | IC₅₀ | |
|---|---|---|
| Number | (nM) | n |
| 19 | 448 (±186) | 9 |
| 84 | 386 (±98) | 16 |
| 20 | 265 (±58) | 2 |
| 85 | 397 (±16) | 2 |
| 21 | 474 | 1 |
| 22 | 1490 | 1 |
| 23 | 271 (±53) | 2 |
| 89 | 427 (±222) | 2 |
| 27 | 173 (±12) | 5 |
| 86 | 184 (±86) | 2 |
| 35 | 391 | 1 |
| 36 | 204 (±116) | 4 |
| 43 | 103 | 1 |
| 87 | 724 | 1 |
| 44 | 291 | 1 |
| 45 | 171 (±78) | 8 |
| 88 | 416 (±159) | 5 |
| 46 | 228 | 1 |
| 47 | 408 (±171) | 10 |
| 48 | 972 (±503) | 4 |
| 49 | 116 (±87) | 3 |
| 50 | 201 (±123) | 4 |
| 51 | 702 | 1 |
| 61 | 771 | 1 |
| 90 | 1191 | 1 |
| 62 | 826 | 1 |
| 63 | 51 | 1 |
| 64 | 236 (±82) | 2 |
| 65 | 364 | 1 |
| 66 | 143 | 1 |
| 67 | 153 | 1 |
| 68 | 709 | 1 |
| 83 | 2907 (±844) | 2 |
| 69 | 269 | 1 |
| 70 | 192 | 1 |
| 71 | 526 (±38) | 2 |
| 72 | 197 | 1 |
| 73 | 182 | 1 |
| 74 | 216 | 3 |
| 75 | 938 | 1 |
| 76 | 470 | 1 |

### Example 52

### Galectin-3 Activity—Microscale Thermophoresis Assay

The carbohydrate recognition domain of human galectin-3 (Servesto Galectin-3-CRD) was diluted with PBS+0.1% F127 to 40 nM. Separately, a 40 uM stock of Compound 45 in DMSO was diluted down to 4 uM in PBS+0.1% F127 and serially diluted in sixteen steps using a dilution factor of 1 to 2 in 10% DMSO+PBS+0.1% F127. To each sample in the serial dilution of Compound 45, 10 ul of 40 nM Galectin-3-CRD was added, yielding final concentrations of 20 nM galectin-3-CRD and 5% DMSO+PBS+0.1% F127. NT.LabelFree Zero background Standard Treated Capillaries were filled with the prepared solutions then loaded into the Monolith NT Label-Free MST instrument. The reaction was measured with 100% excitation power of LED and medium (40%) MST power. Binding curves and Kd values were generated using MO affinity analysis. Each assay was run in triplicate. Some compounds were subjected to more than one assay run in triplicate. For each compound, the average of all assays and standard deviation between the K_{d} value obtained are reported in the following table.

### Galectin-3 Antagonist Activity (Microscale Thermophoresis Assay)

| Compound | K_{d} |
|---|---|
| Number | (nM) |
| 19 | 23 (±5) |
| 84 | 18 (±7) |
| 20 | 36 (±27) |
| 85 | 12 (±15) |
| 21 | 33 (±11) |
| 22 | 83 (±14) |
| 23 | 1.7 (±1.3) |
| 89 | 70 (±10) |
| 27 | 26 (±7) |
| 86 | 0.4 (±6) |
| 35 | 21 (±10) |
| 36 | 14 (±4) |
| 43 | 12 (±3) |
| 87 | 20 (±7) |
| 44 | 12 (±8) |
| 45 | 8.9 (±4) |
| 88 | 67 (±16) |
| 46 | 2.9 (±1.9) |
| 47 | 11 (±3) |
| 48 | 46 (±21) |
| 49 | 18 (±5) |
| 50 | 10 (±3) |
| 51 | 17 (±6) |
| 61 | 10 (±5) |
| 90 | 25 (±8) |
| 62 | 11 (±4) |
| 63 | 22 (±6) |
| 64 | 18 (±7) |
| 65 | 13 (±6) |
| 66 | 3.6 (±2.6) |
| 67 | 2.2 (±1.3) |
| 68 | 87 (±18) |
| 83 | 570 (±76) |
| 69 | 32 (±8) |
| 70 | 20 (±10) |
| 71 | 9 (±3) |
| 72 | 9 (±4) |
| 73 | 8 (±4) |
| 74 | 20 (±8) |
| 75 | 21 (±9) |
| 76 | 31 (±12) |

1. At least one compound chosen from compounds of Formula (I): 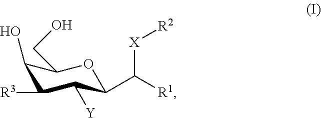
prodrugs of compounds of Formula (I), and pharmaceutically acceptable salts of any of the foregoing, wherein:
R¹ is chosen from —CN, —CH₂CN, and —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H, C₁₋₈ alkyl, C₁₋₈ haloalkyl, C₁₋₈ deuteroalkyl, C₂₋₁₂ heterocyclyl, C₆₋₁₈ aryl, and C₁₋₁₃ heteroaryl groups, wherein the C₂₋₁₂ heterocyclyl, C₆₋₁₈ aryl, and C₁₋₁₃ heteroaryl groups are optionally substituted with one or more groups independently chosen from halo, C₁₋₈ alkyl, C₁₋₈ hydroxyalkyl, C₁₋₈ haloalkyl, C₆₋₁₈ aryl, —OT¹, —C(═O)OT¹, —C(═O)NT¹T², —CN, —ST¹, —S(O)T¹, and —SO₂T¹ groups, wherein T¹ and T², which may be identical or different, are independently chosen from H, C₁₋₈ alkyl, and C₁₋₈ haloalkyl groups, or T¹ and T² join together along with the nitrogen atom to which they are attached to form a ring, or Z¹ and Z² join together along with the nitrogen atom to which they are attached to form a ring;
R² is chosen from C₇₋₁₉ arylalkyl groups substituted with one or more bromo and optionally substituted with one or more groups independently chosen from fluoro, chloro, C₁₋₈ alkyl, C₁₋₈ hydroxyalkyl, C₁₋₈ haloalkyl, C₆₋₁₈ aryl, —OZ³, —C(═O)OZ³, —C(═O)NZ³Z⁴, and —SO₂Z³ groups, wherein Z³ and Z⁴, which may be identical or different, are independently chosen from H, C₁₋₈ alkyl, and C₁₋₈ haloalkyl groups, or Z³ and Z⁴ join together along with the nitrogen atom to which they are attached to form a ring;
R³ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R⁴, C₁₋₈ alkyl, C₁₋₈ haloalkyl, —C(═O)OZ⁵, and —C(═O)NZ⁵Z⁶ groups, wherein R⁴ is independently chosen from C₆₋₁₈ aryl groups optionally substituted with one or more groups independently chosen from halo, C₁₋₈ alkyl, —OZ⁷, —C(═O)OZ⁷, and —C(═O)NZ⁷Z⁸ groups, wherein Z⁵, Z⁶, Z⁷, and Z⁸, which may be identical or different, are independently chosen from H and C₁₋₈ alkyl groups, or Z⁵ and Z⁶ join together along with the nitrogen atom to which they are attached to form a ring and/or Z⁷ and Z⁸ join together along with the nitrogen atom to which they are attached to form a ring;
X is chosen from —O—, —S—, —CH₂—, and —N(R⁵)—, wherein R⁵ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
Y is chosen from H, halo, and —OZ⁹ groups, wherein Z⁹ is chosen from H and C₁₋₈ alkyl, C₁₋₈ haloalkyl, and C₁₋₈ deuteroalkyl groups; and
wherein each of Z¹, Z², Z³, Z⁴, Z⁵, Z⁶, Z⁷, Z⁸, and Z⁹ is optionally substituted with one or more groups independently chosen from halo and —OR⁶ groups, wherein R⁶ is independently chosen from H and C₁₋₈ alkyl groups.

2. The at least one compound according to claim 1 chosen from compounds of Formula (IA): 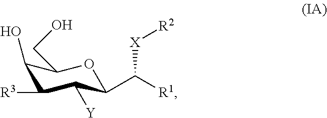
prodrugs of compounds of Formula (IA), and pharmaceutically acceptable salts of any of the foregoing.

3. The at least one compound according to claim 2 chosen from compounds of Formula (IA).

4. The at least one compound according to claim 2, wherein R¹ is chosen from —CN, —CH₂CN, and —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H, C₁₋₈ alkyl, C₁₋₈ haloalkyl, and C₁₋₈ deuteroalkyl groups, or Z¹ and Z² join together along with the nitrogen atom to which they are attached to form a ring.

5. The at least one compound according to claim 2, wherein R¹ is chosen from —C(═O)Q groups, wherein Q is chosen from —OZ¹, —NHOH, —NHOCH₃, —NHCN, and —NZ¹Z² groups, wherein Z¹ and Z², which may be identical or different, are independently chosen from H, C₁₋₈ alkyl, C₁₋₈ haloalkyl, and C₁₋₈ deuteroalkyl groups, or Z¹ and Z² join together along with the nitrogen atom to which they are attached to form a ring.

6. The at least one compound according to claim 5, wherein Q is chosen from —OZ¹ and —NZ¹Z² groups.

7. The at least one compound according to claim 5, wherein Q is chosen from —OZ¹ groups, wherein Z¹ is chosen from H and C₁₋₆ alkyl groups.

8. The at least one compound according to claim 2, wherein R¹ is chosen from

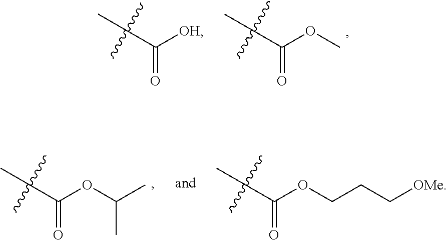

9. The at least one compound according to claim 5, wherein Q is chosen from —NZ¹Z² groups, wherein Z and Z², which may be identical or different, are independently chosen from H and C₁₋₆ alkyl groups.

10. The at least one compound according to claim 2, wherein R¹ is chosen from

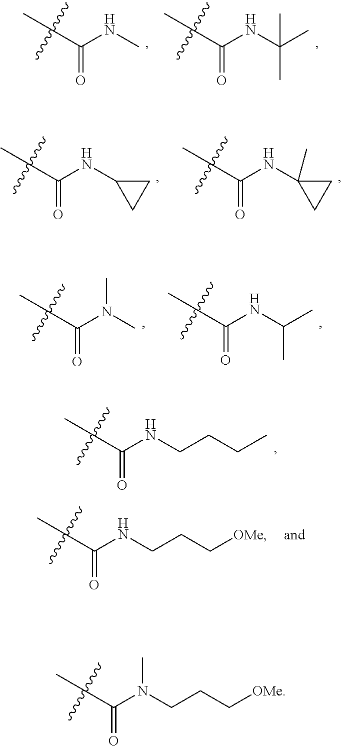

11. The at least one compound according to claim 5, wherein Q is chosen from —NZ¹Z² groups, wherein Z¹ and Z², which may be identical or different, are independently chosen from H and C₁₋₆ haloalkyl groups.

12. The at least one compound according to claim 2, wherein R¹ is chosen from

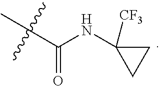 [SMILES: *C(=O)NC1(C(F)(F)F)CC1]

13. The at least one compound according to claim 5, wherein Q is chosen from —NZ¹Z² groups, wherein Z and Z², which may be identical or different, are independently chosen from H and C₁₋₆ deuteroalkyl groups.

14. The at least one compound according to claim 2, wherein R¹ is chosen from

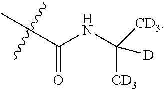 [SMILES: *C(=O)NC([2H])(C([2H])([2H])[2H])C([2H])([2H])[2H]]

15. The at least one compound according to claim 2, wherein R¹ is chosen from —C(═O)Q groups, wherein Q is chosen from —NZ¹Z² groups, wherein Z¹ and Z², which may be identical or different, are independently chosen from H, C₁₋₈ alkyl, C₁₋₈ haloalkyl, C₁₋₈ deuteroalkyl, C₂₋₁₂ heterocyclyl, C₆₋₁₈ aryl, and C₁₋₁₃ heteroaryl groups

16. The at least one compound according to claim 2, wherein R¹ is chosen from —C(═O)Q groups, wherein Q is chosen from —NZ¹Z² groups, wherein Z¹ and Z², which may be identical or different, are independently chosen from H, C₁₋₈ alkyl, C₂₋₁₂ heterocyclyl, C₆₋₁₈ aryl, and C₁₋₁₃ heteroaryl groups.

17. The at least one compound according to claim 2, wherein R¹ is chosen from —C(═O)Q groups, wherein Q is chosen from —NZ¹Z² groups, wherein Z¹ is H and Z² is chosen from C₂₋₁₂ heterocyclyl, C₆₋₁₈ aryl, and C₁₋₁₃ heteroaryl groups.

18. The at least one compound according to claim 2, wherein R¹ is chosen from

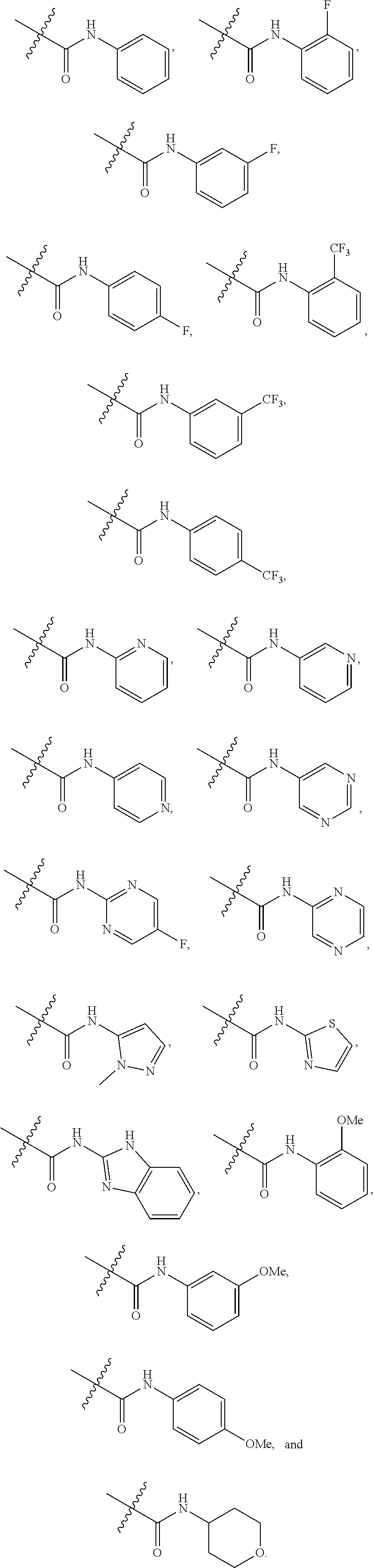

19. The at least one compound according to claim 2, wherein R² is chosen from C₇₋₁₁ arylalkyl groups substituted with one bromo.

20. The at least one compound according to claim 2, wherein R² is chosen from C₇₋₁₁ arylalkyl groups substituted with two bromo.

21. The at least one compound according to claim 2, wherein R² is chosen from C₇₋₁₁ arylalkyl groups substituted with one bromo and one or more halogen independently chosen from fluoro and chloro

22. The at least one compound according to claim 2, wherein R² is chosen from C₇₋₁₁ arylalkyl groups substituted with one bromo and one or more groups independently chosen from C₁₋₈ alkyl groups.

23. The at least one compound according to claim 2, wherein R² is chosen from C₇₋₁₁ arylalkyl groups substituted with one bromo and one or more methyl.

24. The at least one compound according to claim 2, wherein R² is chosen from

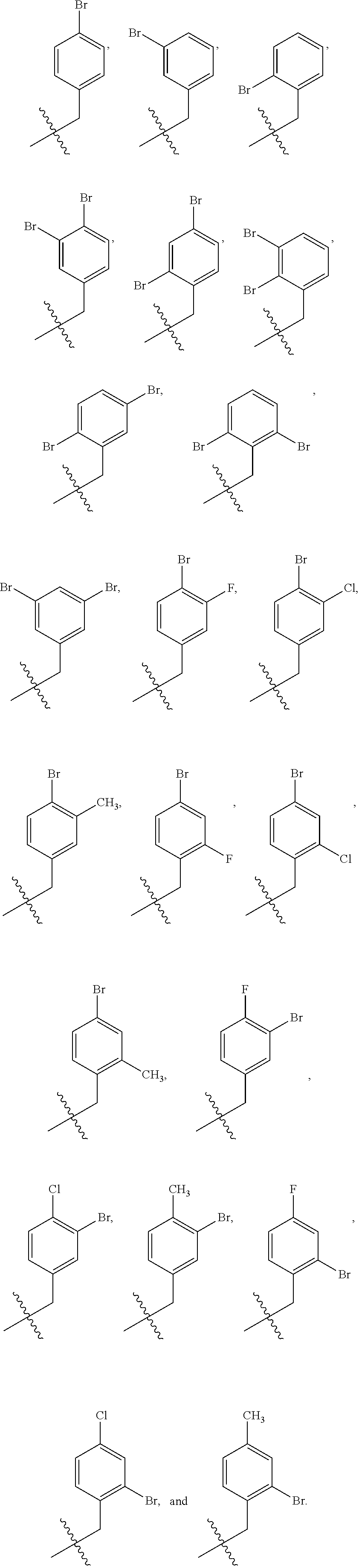

25. The at least one compound according to claim 2, wherein R³ is chosen from C₁₋₁₃ heteroaryl groups.

26. The at least one compound according to claim 25, wherein R³ is chosen from C₁₋₁₃ heteroaryl groups substituted with one or more groups independently chosen from R⁴.

27. The at least one compound according to claim 25, wherein R³ is chosen from

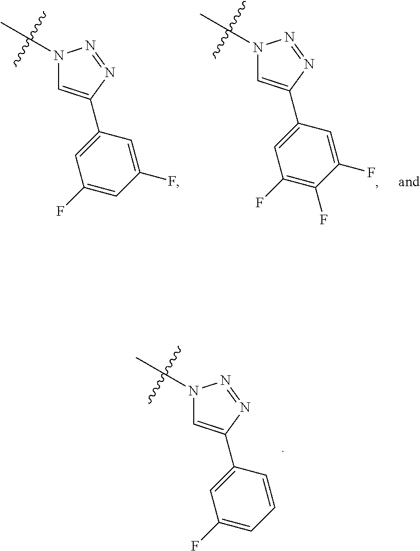 [SMILES: *n1cc(-c2cc(F)cc(F)c2)nn1 ; *n1cc(-c2cc(F)c(F)c(F)c2)nn1 ; *n1cc(-c2cccc(F)c2)nn1]

28. The at least one compound according to claim 2, wherein X is —O—.

29. The at least one compound according to claim 2, wherein X is —S—.

30. The at least one compound according to claim 2, wherein X is —CH₂—.

31. The at least one compound according to claim 2, wherein Y is H.

32. The at least one compound according to claim 2, wherein Y is chosen from halo groups.

33. The at least one compound according to claim 32, wherein Y is fluoro.

34. The at least one compound according to claim 2, wherein Y is chosen from —OZ⁹ groups.

35. The at least one compound according to claim 34, wherein Z⁹ is chosen from C₁₋₈ alkyl, C₁₋₈ haloalkyl, and C₁₋₈ deuteroalkyl groups.

36. The at least one compound according to claim 34, wherein Y is —OCD₃.

37. The at least one compound according to claim 34, wherein Y is —OCF₃.

38. The at least one compound according to claim 34, wherein Y is —OH.

39. The at least one compound according to claim 34, wherein Y is —OMe.

40. The at least one compound according to claim 1 chosen from: 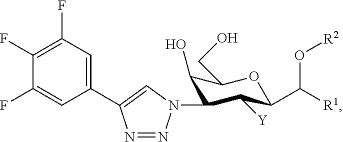
and pharmaceutically acceptable salts thereof, wherein:
R¹ is chosen from 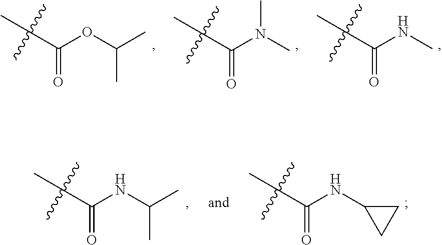
R² is chosen from 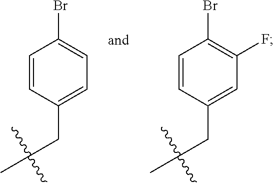 [SMILES: *Cc1ccc(Br)cc1 ; *Cc1ccc(Br)c(F)c1]
and
Y is chosen from —OMe, —OCD₃, and —OCF₃.

41. The at least one compound according to claim 2 chosen from: 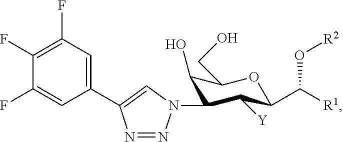
and pharmaceutically acceptable salts thereof, wherein:
R¹ is chosen from 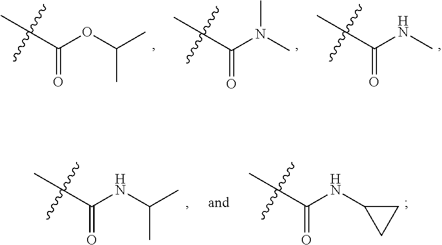
R² is chosen from 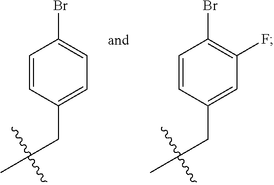 [SMILES: *Cc1ccc(Br)cc1 ; *Cc1ccc(Br)c(F)c1]
and
Y is chosen from —OMe, —OCD₃, and —OCF₃.

42. The at least one compound according to claim 2 chosen from: 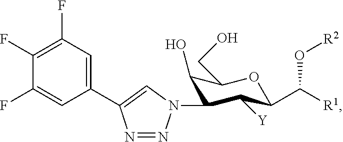
wherein:
R¹ is chosen from 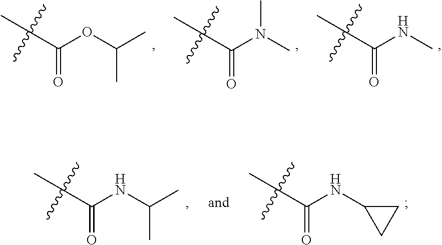
R² is chosen from 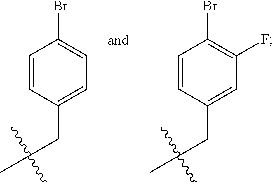 [SMILES: *Cc1ccc(Br)cc1 ; *Cc1ccc(Br)c(F)c1]
and
Y is chosen from —OMe, —OCD₃, and —OCF₃.

43. The at least one compound according to claim 2, wherein the compound is:

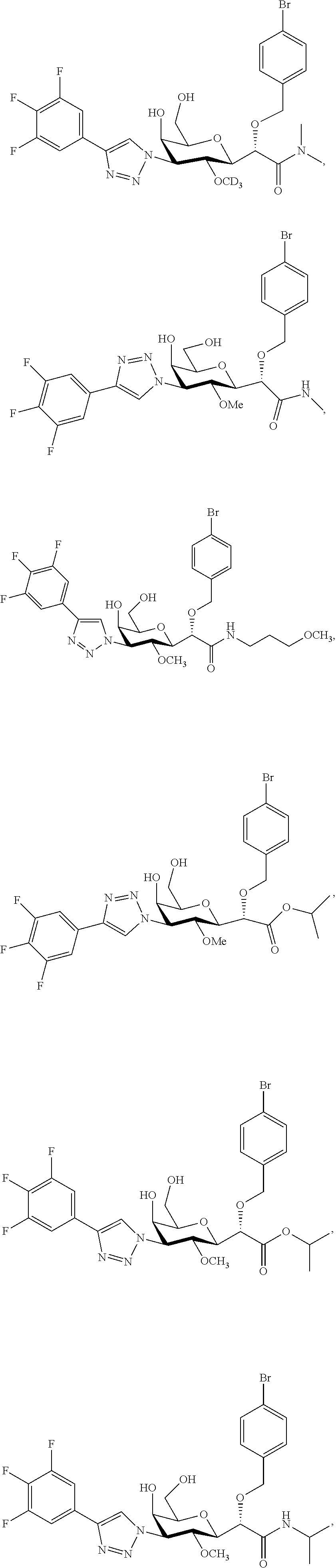

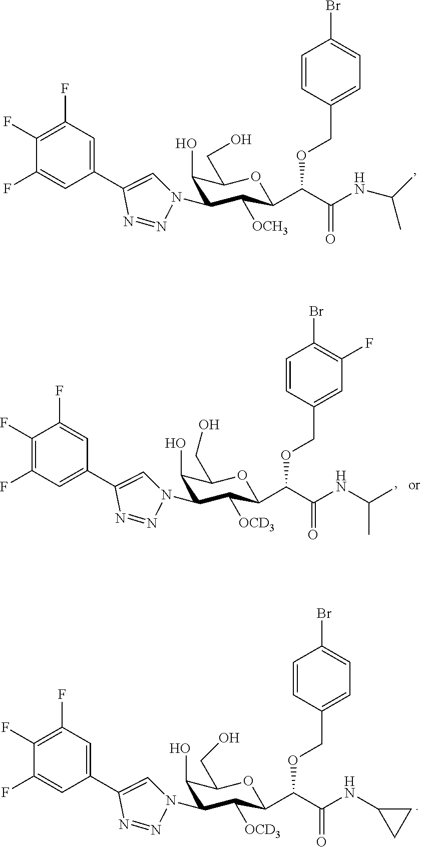

44. The at least one compound according to claim 2, wherein the compound is:

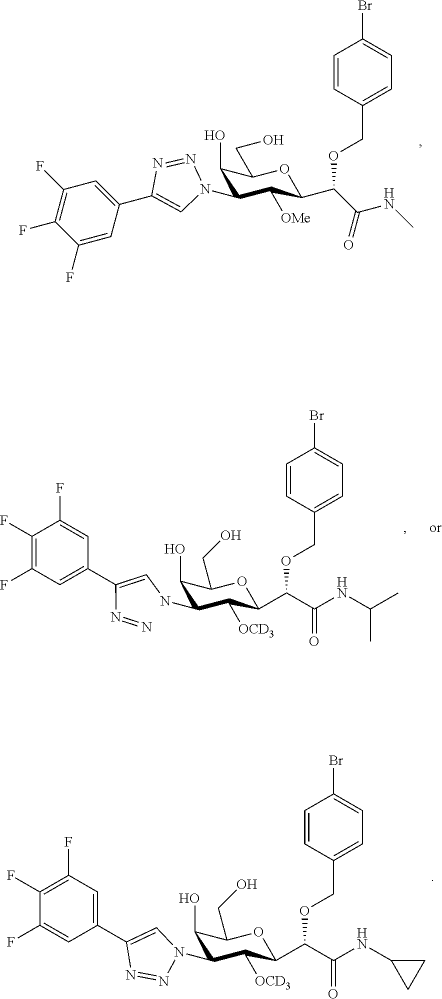

45. The at least one compound according to claim 2, wherein the compound is:

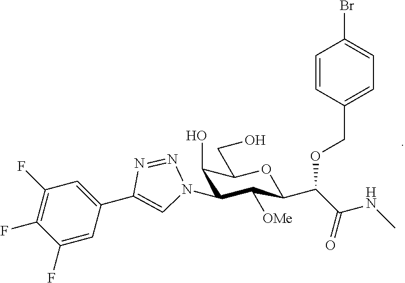

46. The at least one compound according to claim 2, wherein the compound is:

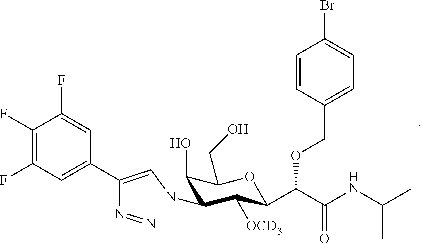

47. The at least one compound according to claim 2, wherein the compound is:

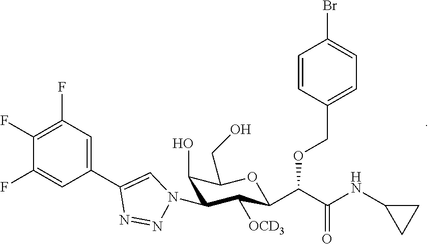

48. A composition comprising the at least one compound of claim 2 and at least one additional pharmaceutically acceptable ingredient.

49. A method for treatment and/or prevention of at least one disease, disorder, and/or condition where inhibition of galectin-3 mediated functions is useful, the method comprising administering to a subject in need thereof an effective amount of at least one compound of claim 41.

50. A method for treatment and/or prevention of at least one inflammatory disease, disorder, and/or condition, the method comprising administering to a subject in need thereof an effective amount of at least one compound of claim 41.

51. A method for treatment and/or prevention of cancer, the method comprising administering to a subject in need thereof an effective amount of at least one compound of claim 41.

52. The method according to claim 51, wherein the cancer is chosen from solid tumor cancers.

53. The method according to claim 51, wherein the cancer is chosen from bone cancers, colorectal cancers, and pancreatic cancers.

54. The method according to claim 51, wherein the cancer is chosen from liquid tumor cancers.

55. The method according to claim 51, wherein the cancer is chosen from acute myelogenous leukemia, acute lymphoblastic leukemia, chronic myelogenous leukemia, and multiple myeloma.

56. A method for treatment and/or prevention of cancer, the method comprising administering to a subject in need thereof (a) an effective amount of at least one compound of claim 41 and (b) at least one therapy chosen from (i) chemotherapy and (ii) radiotherapy.

57. A method for treatment and/or prevention of metastasis of cancer cells, the method comprising administering to a subject in need thereof an effective amount of at least one compound of claim 41.

58. A method for inhibiting infiltration of cancer cells into the liver, lymph nodes, lung, bone, and/or bone marrow, the method comprising administering to a subject in need thereof an effective amount of at least one compound of claim 41.

59. A method for enhancing hematopoietic stem cell survival, the method comprising administering to a subject in need thereof an effective amount of at least one compound of claim 41.

60. The method according to claim 59, wherein the subject has cancer and has received or will receive chemotherapy and/or radiotherapy.

61. A method for mobilizing cells from the bone marrow, the method comprising administering to a subject in need thereof an effective amount of at least one compound of claim 41.

62. The method according to claim 61, wherein the cells are chosen from hematopoietic cells and tumor cells.

63. A method for treatment and/or prevention of thrombosis, the method comprising administering to a subject in need thereof an effective amount of at least one compound of claim 41.

64. A method for treatment and/or prevention of at least one cardiovascular disease or complications associated therewith, the method comprising administering to a subject in need thereof an effective amount of at least one compound of claim 41.

65. The method according to claim 64, wherein the at least one cardiovascular disease is chosen from atherosclerosis and myocardial infarction.

66. A method of inhibiting rejection of a transplanted tissue in a subject, wherein said subject is a recipient of the transplanted tissue, the method comprising administering to a subject in need thereof an effective amount of at least one compound of claim 41.

67. A method for treatment and/or prevention of graft versus host disease or complications associated therewith, the method comprising administering to a subject in need thereof an effective amount of at least one compound of claim 41.

68. A method for treatment and/or prevention of pathological angiogenesis, the method comprising administering to a subject in need thereof an effective amount of at least one compound of claim 41.

69. The method according to claim 68, wherein the pathological angiogenesis occurs in the eye.

70. The method according to claim 68, wherein the pathological angiogenesis occurs in a subject with cancer.

71. A method for treatment and/or prevention of an epileptic syndrome, the method comprising administering to a subject in need thereof an effective amount of at least one compound of claim 41.

72. A method for treatment and/or prevention of neurodegeneration, the method comprising administering to a subject in need thereof an effective amount of at least one compound of claim 41.

73. The method according to claim 72, wherein the neurodegenerative disease is an α-synucleinopathy.

74. A method for treatment and/or prevention of fibrosis, the method comprising administering to a subject in need thereof an effective amount of at least one compound of claim 41.

75. The method according to claim 74, wherein the fibrosis is pulmonary fibrosis.

76. The method according to claim 74, wherein the fibrosis is cardiac fibrosis.

77. A method for treatment and/or prevention of liver disorders or complications associated therewith, the method comprising administering to a subject in need thereof an effective amount of at least one compound of claim 41.

78. The method according to claim 77, wherein the liver disorder is nonalcoholic steatohepatitis.

79. The at least one compound according to claim 2, wherein the compound is: [chemical structure]
